(19) United States
(12) Patent Application Publication
(10) Pub. No.: US 2018/0021454 A1
(43) Pub. Date: Jan. 25, 2018

(54) TARGETED CONJUGATES ENCAPSULATED IN PARTICLES AND FORMULATIONS THEREOF

(71) Applicant: TARVEDA THERAPEUTICS, INC., Watertown, MA (US)

(72) Inventors: Mark T. Bilodeau, Concord, MA (US); Sudhakar Kadiyala, Newton, MA (US); Rajesh R. Shinde, Waltham, MA (US); Brian H. White, Malden, MA (US); Richard Wooster, Natick, MA (US); Benoît Moreau, Newton, MA (US)

(21) Appl. No.: 15/695,214

(22) Filed: Sep. 5, 2017

Related U.S. Application Data
(63) Continuation of application No. 14/949,138, filed on Nov. 23, 2015.
Application No. 14/949,138 is a continuation of application No. 14/144,263, filed on Dec. 30, 2013, now abandoned.
(60) Provisional application No. 61/746,866, filed on Dec. 28, 2012.

(51) Int. Cl. A61K 31/519; A61K 31/282; A61K 9/16; A61K 31/337

(52) U.S. Cl. CPC A61K 47/6929; A61K 31/519; A61K 31/337; A61K 31/282; A61K 9/1647; A61K 9/16; A61K 47/64; A61K 47/551; A61K 47/542; Y10T 428/2982

(57) ABSTRACT

Particles, including nanoparticles and microparticles, and pharmaceutical formulations thereof, containing conjugates of an active agent such as a therapeutic, prophylactic, or diagnostic agent attached to a targeting moiety via a linker have been designed which can provide improved temporospatial delivery of the active agent and/or improved biodistribution. Methods of making the conjugates, the particles, and the formulations thereof are provided. Methods of administering the formulations to a subject in need thereof are provided, for example, to treat or prevent cancer or infectious diseases.

### CROSS-REFERENCE TO RELATED APPLICATIONS

This application is a continuation of U.S. application Ser. No. 14/949,138 filed Nov. 23, 2015, which is a continuation of U.S. application Ser. No. 14/144,263 filed Dec. 30, 2013, which claims priority to U.S. Provisional Application No. 61/746,866 filed Dec. 28, 2012, the contents of each of which are herein incorporated by reference in their entirety.

### FIELD OF THE INVENTION

This invention is generally in the field of targeting ligands and conjugates thereof for drug delivery.

### BACKGROUND OF THE INVENTION

Developments in nanomedicine are directed towards improving the pharmaceutical properties of the drugs and enhancing the targeted delivery in a cell-specific manner. Several cell-specific drugs are known in literature, and include monoclonal antibodies, aptamers, peptides, and small molecules. Despite some of the potential advantages of these drugs, a number of problems have limited their clinical application, including size, stability, manufacturing cost, immunogenicity, poor pharmacokinetics and other factors.

Nanoparticulate drug delivery systems are attractive for systemic drug delivery because of their ability to prolong drug circulation half-life, reduce non-specific uptake, and better accumulate at the tumors through an enhanced permeation and retention (EPR) effect. As a result, several therapeutic formulations such as DOXIL® (liposomal encapsulated doxyrubicin) and ABRAXANE® (albumin bound paclitaxel nanoparticles) are used as the frontline therapies.

The development of nanotechnologies for effective delivery of drugs or drug candidates to specific diseased cells and tissues, e.g., to cancer cells, in specific organs or tissues, in a temporospatially regulated manner can potentially overcome the therapeutic challenges faced to date, such as systemic toxicity. However, while targeting of the delivery system delivers drug to the site where therapy is needed, the drug that is released may not remain in the region of the targeted cells in efficacious amounts. Accordingly, there is a need in the art for improved drug targeting and delivery.

It is therefore an object of the invention to provide improved compounds, compositions, and formulations for temporospatial drug delivery.

It is further an object of the invention to provide methods of making improved compounds, compositions, and formulations for temporospatial drug delivery.

It is also an object of the invention to provide methods of administering the improved compounds, compositions, and formulations to individuals in need thereof.

### SUMMARY OF THE INVENTION

Particles, including polymeric nanoparticles and microparticles, and pharmaceutical formulations thereof, containing conjugates of an active agent such as a therapeutic, prophylactic, or diagnostic agent attached to a targeting moiety via a linker have been designed which can provide improved temporospatial delivery of the active agent and/or improved biodistribution. Methods of making the conjugates, the particles, and the formulations thereof are provided. Methods of administering the formulations to a subject in need thereof are provided, for example, to treat or prevent cancer or infectious diseases.

The conjugates are released after administration of the particles. The targeted drug conjugates utilize active molecular targeting in combination with enhanced permeability and retention effect (EPR) and improved overall biodistribution of the particles to provide greater efficacy and tolerability as compared to administration of targeted particles or encapsulated untargeted drug.

The conjugates include a targeting ligand and an active agent connected by a linker, wherein the conjugate in some embodiments has the formula:

(X—Y—Z)

wherein X is a targeting moiety; Y is a linker; and Z is an active agent.

One ligand can be conjugated to two or more active agents where the conjugate has the formula: X—(Y—Z)ₙ. In other embodiments, one active agent molecule can be linked to two or more ligands wherein the conjugate has the formula: (X—Y)ₙ—Z. n is an integer equal to or greater than 1.

The targeting moiety, X, can be a molecule such as a peptide such as somatostatin, octeotide, epidermal growth factor (“EGF”) or RGD-containing peptides; an aptamer such as RNA, DNA or an artificial nucleic acid; a small molecule; a carbohydrate such as mannose, galactose or arabinose; a vitamin such as ascorbic acid, niacin, pantothenic acid, carnitine, inositol, pyridoxal, lipoic acid, folic acid (folate), riboflavin, biotin, vitamin B₁₂, vitamin A, E, and K; a protein such as thrombospondin, tumor necrosis factors (TNF), annexin V, an interferon, angiostatin, endostatin, cytokine, transferrin, GM-CSF (granulocyte-macrophage colony-stimulating factor), or growth factors such as vascular endothelial growth factor (VEGF), hepatocyte growth factor (HGF), (platelet-derived growth factor (PDGF), basic fibroblast growth factor (bFGF), and epidermal growth factor (EGF). In a preferred embodiment, the targeting moiety is an antibody fragment, RGD peptide, folic acid or prostate specific membrane antigen (PSMA).

The linker, Y, is bound to an active agent and a targeting ligand to form a conjugate. The linker can contain a C₁-C₁₀ straight chain alkyl, C₁-C₁₀ straight chain O-alkyl, C₁-C₁₀ straight chain substituted alkyl, C₁-C₁₀ straight chain substituted O-alkyl, C₄-C₁₃ branched chain alkyl, C₄-C₁₃ branched chain O-alkyl, C₂-C₁₂ straight chain alkenyl, C₂-C₁₂ straight chain O-alkenyl, C₃-C₁₂ straight chain substituted alkenyl, C₃-C₁₂ straight chain substituted O-alkenyl, polyethylene glycol, polylactic acid, polyglycolic acid, poly(lactide-co-glycolide), polycarprolactone, polycyanoacrylate, ketone, aryl, heterocyclic, succinic ester, amino acid, aromatic group, ether, crown ether, ester, urea, thiourea, amide, purine, pyrimidine, bypiridine, indole derivative acting as a cross linker, chelator, aldehyde, ketone, bisamine, bis alcohol, heterocyclic ring structure, azirine, disulfide, thioether, hydrazone and combinations thereof. For example, the linker can be a C₃ straight chain alkyl or a ketone. The linker can release the active agent at the desired site of release.

The active agent, Z, is preferably a chemotherapeutic agent, antimicrobial, or combination thereof. For example, the active agent, Z, can be cabazitaxel, a platinum(IV) complex, or analogue or derivative thereof.

In one embodiment, a RGD peptide-SS-cabazitaxel conjugate of Formula I is provided as follows.

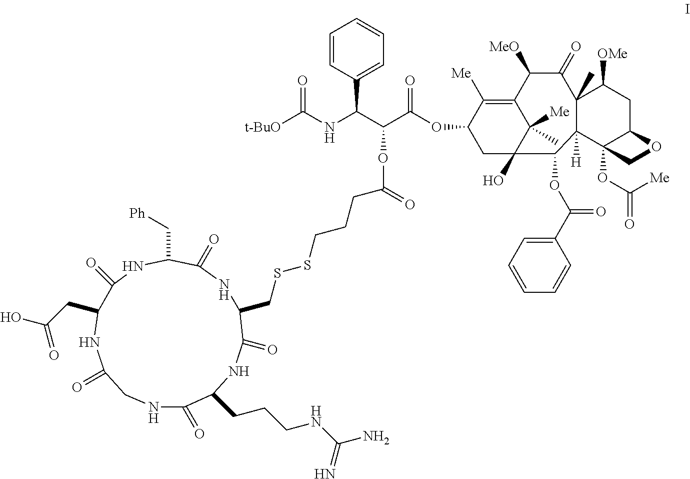

In another embodiment, a folate-platinum(IV) conjugate of Formula II is provided as follows.

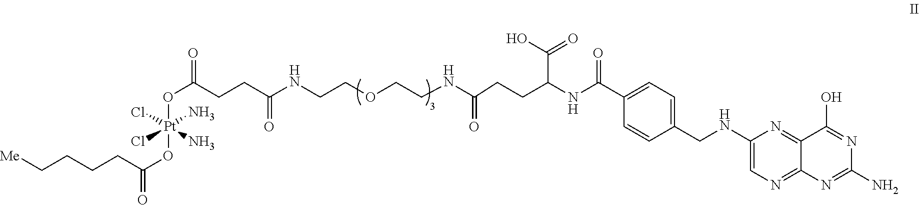

In a further embodiment, a PSMA-cabazitaxel conjugate of Formula III is provided as follows.

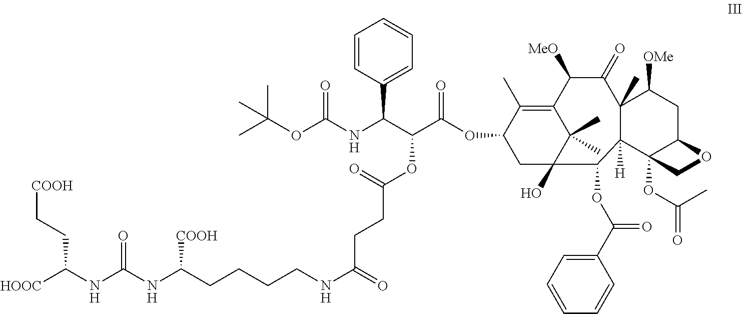

In another embodiment, a PSMA-platinum(IV) conjugate is provided as follows.

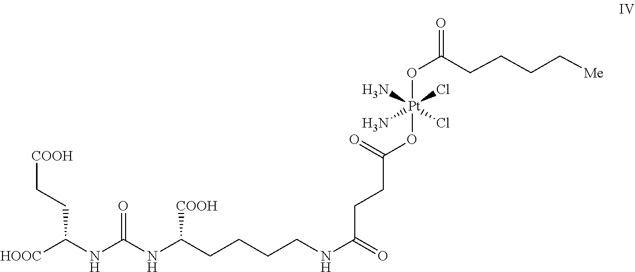

In yet another embodiment, a folate-cabazitaxel conjugate is provided as follows:

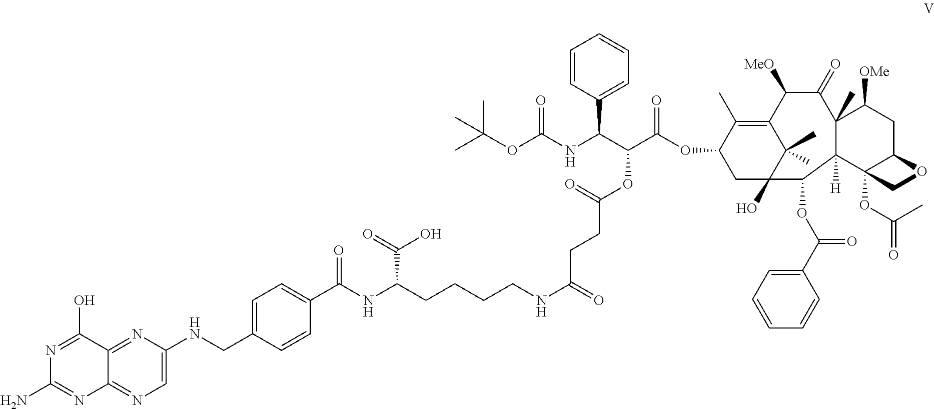

In yet another embodiment, a PSMA-cabazitaxel conjugate is provided as follows:

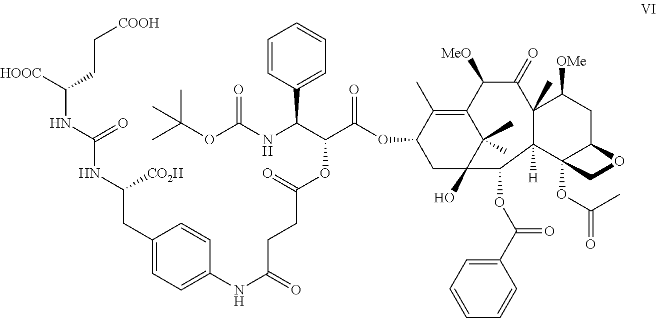

In yet another embodiment, a PSMA-cabazitaxel conjugate is provided as follows:

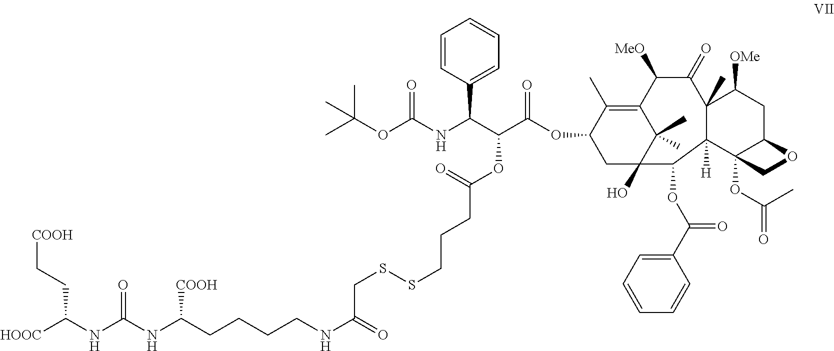

In yet another embodiment, a folate-Pt(IV) conjugate is provided as follows:

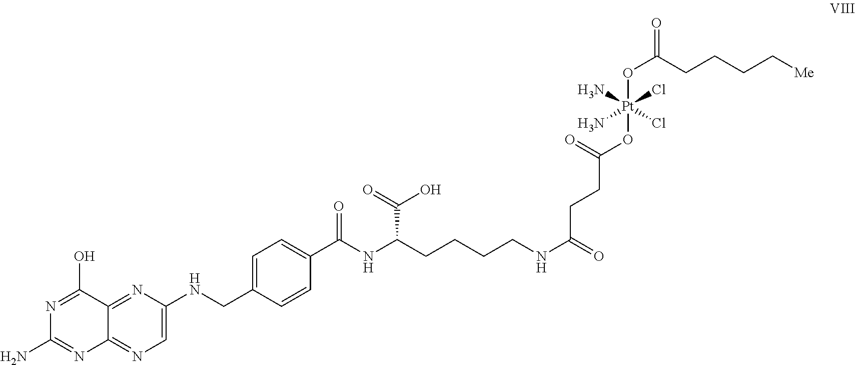

In yet another embodiment, a Pt(IV)-di-folate conjugate is provided as follows:

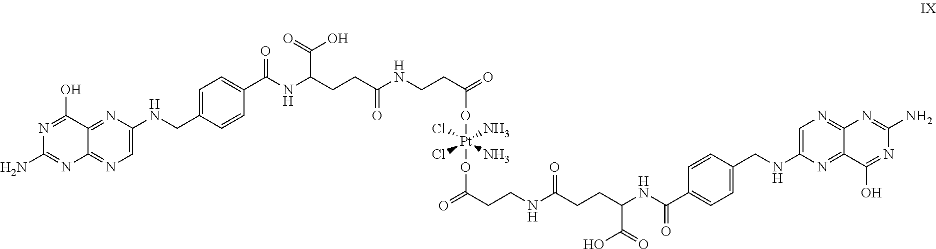

In yet another embodiment, a PSMA-di-Pt(IV) conjugate is provided as follows:

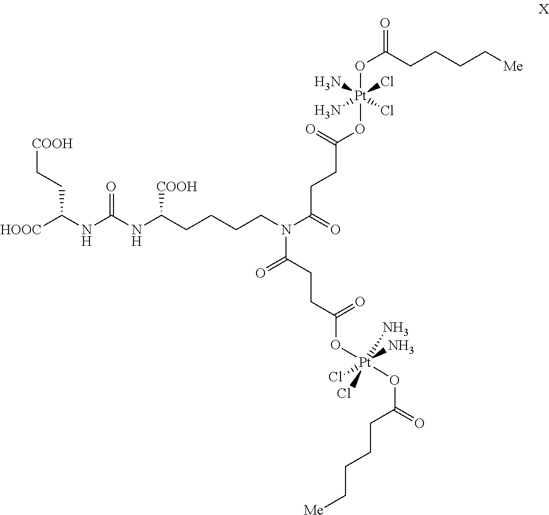

In yet another embodiment, a RGD peptide-SS-cabazitaxel conjugate is provided as follows:

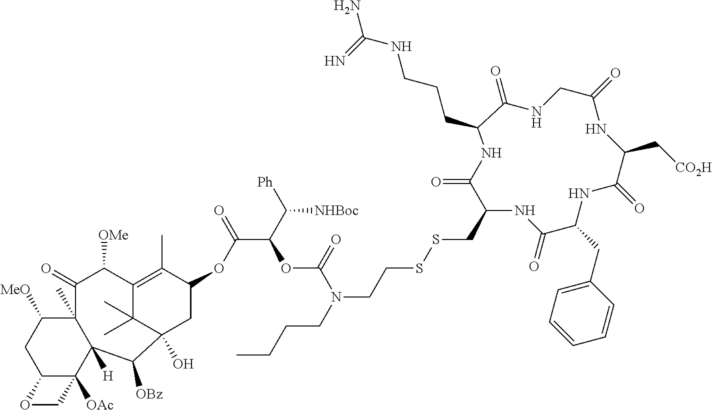

Pharmaceutical formulations are provided containing the nanoparticulate conjugates described herein, or pharmaceutically acceptable salts thereof, in a pharmaceutically acceptable vehicle. In the preferred embodiment, the formulations are administered by injection.

Methods of making the conjugates and particles containing the conjugates are provided. Methods are also provided for treating a disease or condition, the method comprising administering a therapeutically effective amount of the particles containing a conjugate to a subject in need thereof. In a preferred embodiment, the conjugates are targeted to a cancer or proliferative disease including lymphoma, renal cell carcinoma, leukemia, prostate cancer, lung cancer, pancreatic cancer, melanoma, colorectal cancer, ovarian cancer, breast cancer, glioblastoma multiforme, stomach cancer, liver cancer, sarcoma, bladder cancer, testicular cancer, esophageal cancer, head and neck cancer, endometrial cancer and leptomeningeal carcinomatosis.

### BRIEF DESCRIPTION OF THE DRAWINGS

FIG. 1 is a graph of the blood plasma concentration (μM) of the cabazitaxel-RDG conjugate of Example 2 as a function of time (hours) after tail vein injection in rats. The formulations injected contained either the free cabazitaxel-RDG conjugate or the cabazitaxel-RDG nanoparticles of Example 3.

### DETAILED DESCRIPTION OF THE INVENTION

### I. Definitions

The terms “subject” or “patient”, as used herein, refer to any organism to which the particles may be administered, e.g., for experimental, therapeutic, diagnostic, and/or prophylactic purposes. Typical subjects include animals (e.g., mammals such as mice, rats, rabbits, non-human primates, and humans) and/or plants.

The terms “treating” or “preventing”, as used herein, can include preventing a disease, disorder or condition from occurring in an animal which may be predisposed to the disease, disorder and/or condition but has not yet been diagnosed as having it; inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, such as treating the pain of a subject by administration of an analgesic agent even though such agent does not treat the cause of the pain.

A “target”, as used herein, shall mean a site to which targeted constructs bind. A target may be either in vivo or in vitro. In certain embodiments, a target may be cancer cells found in leukemias or tumors (e.g., tumors of the brain, lung (small cell and non-small cell), ovary, prostate, breast and colon as well as other carcinomas and sarcomas). In other embodiments, a target may be a site of infection (e.g., by bacteria, viruses (e.g., HIV, herpes, hepatitis)) and pathogenic fungi (e.g., Candida sp.). Certain target infectious organisms include those that are drug resistant (e.g., Enterobacteriaceae, Enterococcus, Haemophilus influenza, Mycobacterium tuberculosis, Neisseria gonorrhoeae, Plasmodium falciparum, Pseudomonas aeruginosa, Shigella dysenteriae, Staphylococcus aureus, Streptococcus pneumoniae). In still other embodiments, a target may refer to a molecular structure to which a targeting moiety or ligand binds, such as a hapten, epitope, receptor, dsDNA fragment, carbohydrate or enzyme. Additionally, a target may be a type of tissue, e.g., neuronal tissue, intestinal tissue, pancreatic tissue etc.

The “target cells” that may serve as the target for the method or coordination complexes, include prokaryotes and eukaryotes, including yeasts, plant cells and animal cells. The present method may be used to modify cellular function of living cells in vitro, i.e., in cell culture, or in vivo, in which the cells form part of or otherwise exist in plant tissue or animal tissue. Thus, the target cells may include, for example, the blood, lymph tissue, cells lining the alimentary canal, such as the oral and pharyngeal mucosa, cells forming the villi of the small intestine, cells lining the large intestine, cells lining the respiratory system (nasal passages/lungs) of an animal (which may be contacted by inhalation of the subject invention), dermal/epidermal cells, cells of the vagina and rectum, cells of internal organs including cells of the placenta and the so-called blood/brain barrier, etc.

The term “therapeutic effect” is art-recognized and refers to a local or systemic effect in animals, particularly mammals, and more particularly humans caused by a pharmacologically active substance. The term thus means any substance intended for use in the diagnosis, cure, mitigation, treatment or prevention of disease or in the enhancement of desirable physical or mental development and conditions in an animal or human.

The term “modulation” is art-recognized and refers to up regulation (i.e., activation or stimulation), down regulation (i.e., inhibition or suppression) of a response, or the two in combination or apart.

“Parenteral administration”, as used herein, means administration by any method other than through the digestive tract or non-invasive topical or regional routes. For example, parenteral administration may include administration to a patient intravenously, intradermally, intraperitoneally, intrapleurally, intratracheally, intramuscularly, subcutaneously, subjunctivally, by injection, and by infusion.

“Topical administration”, as used herein, means the non-invasive administration to the skin, orifices, or mucosa. Topical administrations can be administered locally, i.e., they are capable of providing a local effect in the region of application without systemic exposure. Topical formulations can provide systemic effect via adsorption into the blood stream of the individual. Topical administration can include, but is not limited to, cutaneous and transdermal administration, buccal administration, intranasal administration, intravaginal administration, intravesical administration, ophthalmic administration, and rectal administration.

“Enteral administration”, as used herein, means administration via absorption through the gastrointestinal tract. Enteral administration can include oral and sublingual administration, gastric administration, or rectal administration.

“Pulmonary administration”, as used herein, means administration into the lungs by inhalation or endotracheal administration. As used herein, the term “inhalation” refers to intake of air to the alveoli. The intake of air can occur through the mouth or nose.

The terms “sufficient” and “effective”, as used interchangeably herein, refer to an amount (e.g., mass, volume, dosage, concentration, and/or time period) needed to achieve one or more desired result(s). A “therapeutically effective amount” is at least the minimum concentration required to effect a measurable improvement or prevention of any symptom or a particular condition or disorder, to effect a measurable enhancement of life expectancy, or to generally improve patient quality of life. The therapeutically effective amount is thus dependent upon the specific biologically active molecule and the specific condition or disorder to be treated. Therapeutically effective amounts of many active agents, such as antibodies, are well known in the art. The therapeutically effective amounts of anionic proteins, protein analogues, or nucleic acids hereinafter discovered or for treating specific disorders with known proteins, protein analogues, or nucleic acids to treat additional disorders may be determined by standard techniques which are well within the craft of a skilled artisan, such as a physician.

The terms “bioactive agent” and “active agent”, as used interchangeably herein, include, without limitation, physiologically or pharmacologically active substances that act locally or systemically in the body. A bioactive agent is a substance used for the treatment (e.g., therapeutic agent), prevention (e.g., prophylactic agent), diagnosis (e.g., diagnostic agent), cure or mitigation of disease or illness, a substance which affects the structure or function of the body, or pro-drugs, which become biologically active or more active after they have been placed in a predetermined physiological environment.

The term “prodrug” refers to an agent, including a nucleic acid or proteins that is converted into a biologically active form in vitro and/or in vivo. Prodrugs can be useful because, in some situations, they may be easier to administer than the parent compound. For example, a prodrug may be bioavailable by oral administration whereas the parent compound is not. The prodrug may also have improved solubility in pharmaceutical compositions compared to the parent drug. A prodrug may be converted into the parent drug by various mechanisms, including enzymatic processes and metabolic hydrolysis. Harper, N.J. (1962) Drug Latentiation in Jucker, ed. Progress in Drug Research, 4:221-294; Morozowich et al. (1977) Application of Physical Organic Principles to Prodrug Design in E. B. Roche ed. Design of Biopharmaceutical Properties through Prodrugs and Analogs, APhA; Acad. Pharm. Sci.; E. B. Roche, ed. (1977) Bioreversible Carriers in Drug in Drug Design, Theory and Application, APhA; H. Bundgaard, ed. (1985) Design of Prodrugs, Elsevier; Wang et al. (1999) Prodrug approaches to the improved delivery of peptide drug, Curr. Pharm. Design. 5(4):265-287; Pauletti et al. (1997) Improvement in peptide bioavailability: Peptidomimetics and Prodrug Strategies, Adv. Drug. Delivery Rev. 27:235-256; Mizen et al. (1998). The Use of Esters as Prodrugs for Oral Delivery of β-Lactam antibiotics, Pharm. Biotech. 11:345-365; Gaignault et al. (1996) Designing Prodrugs and Bioprecursors I. Carrier Prodrugs, Pract. Med. Chem. 671-696; M. Asgharnejad (2000). Improving Oral Drug Transport Via Prodrugs, in G. L. Amidon, P. I. Lee and E. M. Topp, Eds., Transport Processes in Pharmaceutical Systems, Marcell Dekker, p. 185-218; Balant et al. (1990) Prodrugs for the improvement of drug absorption via different routes of administration, Eur. J. Drug Metab. Pharmacokinet., 15(2): 143-53; Balimane and Sinko (1999). Involvement of multiple transporters in the oral absorption of nucleoside analogues, Adv. Drug Delivery Rev., 39(1-3):183-209; Browne (1997). Fosphenytoin (Cerebyx), Clin. Neuropharmacol. 20(1): 1-12; Bundgaard (1979). Bioreversible derivatization of drugs—principle and applicability to improve the therapeutic effects of drugs, Arch. Pharm. Chemi. 86(1): 1-39; H. Bundgaard, ed. (1985) Design of Prodrugs, New York: Elsevier; Fleisher et al. (1996) Improved oral drug delivery: solubility limitations overcome by the use of prodrugs, Adv. Drug Delivery Rev. 19(2): 115-130; Fleisher et al. (1985) Design of prodrugs for improved gastrointestinal absorption by intestinal enzyme targeting, Methods Enzymol. 112: 360-81; Farquhar D, et al. (1983) Biologically Reversible Phosphate-Protective Groups, J. Pharm. Sci., 72(3): 324-325; Han, H. K. et al. (2000) Targeted prodrug design to optimize drug delivery, AAPS PharmSci., 2(1): E6; Sadzuka Y. (2000) Effective prodrug liposome and conversion to active metabolite, Curr. Drug Metab., 1(1):31-48; D. M. Lambert (2000) Rationale and applications of lipids as prodrug carriers, Eur. J. Pharm. Sci., 11 Suppl. 2:S15-27; Wang, W. et al. (1999) Prodrug approaches to the improved delivery of peptide drugs. Curr. Pharm. Des., 5(4):265-87.

The term “biocompatible”, as used herein, refers to a material that along with any metabolites or degradation products thereof that are generally non-toxic to the recipient and do not cause any significant adverse effects to the recipient. Generally speaking, biocompatible materials are materials which do not elicit a significant inflammatory or immune response when administered to a patient.

The term “biodegradable” as used herein, generally refers to a material that will degrade or erode under physiologic conditions to smaller units or chemical species that are capable of being metabolized, eliminated, or excreted by the subject. The degradation time is a function of composition and morphology. Degradation times can be from hours to weeks.

The term “pharmaceutically acceptable”, as used herein, refers to compounds, materials, compositions, and/or dosage forms that are, within the scope of sound medical judgment, suitable for use in contact with the tissues of human beings and animals without excessive toxicity, irritation, allergic response, or other problems or complications commensurate with a reasonable benefit/risk ratio, in accordance with the guidelines of agencies such as the U.S. Food and Drug Administration. A “pharmaceutically acceptable carrier”, as used herein, refers to all components of a pharmaceutical formulation that facilitate the delivery of the composition in vivo. Pharmaceutically acceptable carriers include, but are not limited to, diluents, preservatives, binders, lubricants, disintegrators, swelling agents, fillers, stabilizers, and combinations thereof.

The term “molecular weight”, as used herein, generally refers to the mass or average mass of a material. If a polymer or oligomer, the molecular weight can refer to the relative average chain length or relative chain mass of the bulk polymer. In practice, the molecular weight of polymers and oligomers can be estimated or characterized in various ways including gel permeation chromatography (GPC) or capillary viscometry. GPC molecular weights are reported as the weight-average molecular weight (M_{w}) as opposed to the number-average molecular weight (Mₙ). Capillary viscometry provides estimates of molecular weight as the inherent viscosity determined from a dilute polymer solution using a particular set of concentration, temperature, and solvent conditions.

The term “small molecule”, as used herein, generally refers to an organic molecule that is less than 2000 g/mol in molecular weight, less than 1500 g/mol, less than 1000 g/mol, less than 800 g/mol, or less than 500 g/mol. Small molecules are non-polymeric and/or non-oligomeric.

The term “hydrophilic”, as used herein, refers to substances that have strongly polar groups that readily interact with water.

The term “hydrophobic”, as used herein, refers to substances that lack an affinity for water; tending to repel and not absorb water as well as not dissolve in or mix with water.

The term “lipophilic”, as used herein, refers to compounds having an affinity for lipids.

The term “amphiphilic”, as used herein, refers to a molecule combining hydrophilic and lipophilic (hydrophobic) properties. “Amphiphilic material” as used herein refers to a material containing a hydrophobic or more hydrophobic oligomer or polymer (e.g., biodegradable oligomer or polymer) and a hydrophilic or more hydrophilic oligomer or polymer.

The term “targeting moiety”, as used herein, refers to a moiety that binds to or localizes to a specific locale. The moiety may be, for example, a protein, nucleic acid, nucleic acid analog, carbohydrate, or small molecule. The locale may be a tissue, a particular cell type, or a subcellular compartment. In some embodiments, a targeting moiety can specifically bind to a selected molecule.

The term “reactive coupling group”, as used herein, refers to any chemical functional group capable of reacting with a second functional group to form a covalent bond. The selection of reactive coupling groups is within the ability of the skilled artisan. Examples of reactive coupling groups can include primary amines (—NH₂) and amine-reactive linking groups such as isothiocyanates, isocyanates, acyl azides, NHS esters, sulfonyl chlorides, aldehydes, glyoxals, epoxides, oxiranes, carbonates, aryl halides, imidoesters, carbodiimides, anhydrides, and fluorophenyl esters. Most of these conjugate to amines by either acylation or alkylation. Examples of reactive coupling groups can include aldehydes (—COH) and aldehyde reactive linking groups such as hydrazides, alkoxyamines, and primary amines. Examples of reactive coupling groups can include thiol groups (—SH) and sulfhydryl reactive groups such as maleimides, haloacetyls, and pyridyl disulfides. Examples of reactive coupling groups can include photoreactive coupling groups such as aryl azides or diazirines. The coupling reaction may include the use of a catalyst, heat, pH buffers, light, or a combination thereof.

The term “protective group”, as used herein, refers to a functional group that can be added to and/or substituted for another desired functional group to protect the desired functional group from certain reaction conditions and selectively removed and/or replaced to deprotect or expose the desired functional group. Protective groups are known to the skilled artisan. Suitable protective groups may include those described in Greene, T. W. and Wuts, P. G. M., Protective Groups in Organic Synthesis, (1991). Acid sensitive protective groups include dimethoxytrityl (DMT), tert-butylcarbamate (tBoc) and trifluoroacetyl (tFA). Base sensitive protective groups include 9-fluorenylmethoxycarbonyl (Fmoc), isobutyrl (iBu), benzoyl (Bz) and phenoxyacetyl (pac). Other protective groups include acetamidomethyl, acetyl, tert-amyloxycarbonyl, benzyl, benzyloxycarbonyl, 2-(4-biphεnylyl)-2-propy!oxycarbonyl, 2-bromobenzyloxycarbonyl, tert-butyl₇ tert-butyloxycarbonyl, 1-carbobenzoxamido-2,2.2-trifluoroethyl, 2,6-dichlorobenzyl, 2-(3,5-dimethoxyphenyl)-2-propyloxycarbonyl, 2,4-dinitrophenyl, dithiasuccinyl, formyl, 4-methoxybenzenesulfonyl, 4-methoxybenzyl, 4-methylbenzyl, o-nitrophenylsulfenyl, 2-phenyl-2-propyloxycarbonyl, α-2,4,5-tetramethylbenzyloxycarbonyl, p-toluenesulfonyl, xanthenyl, benzyl ester, N-hydroxysuccinimide ester, p-nitrobenzyl ester, p-nitrophenyl ester, phenyl ester, p-nitrocarbonate, p-nitrobenzylcarbonate, trimethylsilyl and pentachlorophenyl ester.

The term “activated ester”, as used herein, refers to alkyl esters of carboxylic acids where the alkyl is a good leaving group rendering the carbonyl susceptible to nucleophilic attack by molecules bearing amino groups. Activated esters are therefore susceptible to aminolysis and react with amines to form amides. Activated esters contain a carboxylic acid ester group —CO₂R where R is the leaving group.

The term “alkyl” refers to the radical of saturated aliphatic groups, including straight-chain alkyl groups, branched-chain alkyl groups, cycloalkyl (alicyclic) groups, alkyl-substituted cycloalkyl groups, and cycloalkyl-substituted alkyl groups.

In some embodiments, a straight chain or branched chain alkyl has 30 or fewer carbon atoms in its backbone (e.g., C₁-C₃₀ for straight chains, C₃-C₃₀ for branched chains), 20 or fewer, 12 or fewer, or 7 or fewer. Likewise, in some embodiments cycloalkyls have from 3-10 carbon atoms in their ring structure, e.g. have 5, 6 or 7 carbons in the ring structure. The term “alkyl” (or “lower alkyl”) as used throughout the specification, examples, and claims is intended to include both “unsubstituted alkyls” and “substituted alkyls”, the latter of which refers to alkyl moieties having one or more substituents replacing a hydrogen on one or more carbons of the hydrocarbon backbone. Such substituents include, but are not limited to, halogen, hydroxyl, carbonyl (such as a carboxyl, alkoxycarbonyl, formyl, or an acyl), thiocarbonyl (such as a thioester, a thioacetate, or a thioformate), alkoxyl, phosphoryl, phosphate, phosphonate, a hosphinate, amino, amido, amidine, imine, cyano, nitro, azido, sulfhydryl, alkylthio, sulfate, sulfonate, sulfamoyl, sulfonamido, sulfonyl, heterocyclyl, aralkyl, or an aromatic or heteroaromatic moiety.

Unless the number of carbons is otherwise specified, “lower alkyl” as used herein means an alkyl group, as defined above, but having from one to ten carbons, or from one to six carbon atoms in its backbone structure. Likewise, “lower alkenyl” and “lower alkynyl” have similar chain lengths. Throughout the application, preferred alkyl groups are lower alkyls. In some embodiments, a substituent designated herein as alkyl is a lower alkyl.

It will be understood by those skilled in the art that the moieties substituted on the hydrocarbon chain can themselves be substituted, if appropriate. For instance, the substituents of a substituted alkyl may include halogen, hydroxy, nitro, thiols, amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Cycloalkyls can be substituted in the same manner.

The term “heteroalkyl”, as used herein, refers to straight or branched chain, or cyclic carbon-containing radicals, or combinations thereof, containing at least one heteroatom. Suitable heteroatoms include, but are not limited to, O, N, Si, P, Se, B, and S, wherein the phosphorous and sulfur atoms are optionally oxidized, and the nitrogen heteroatom is optionally quaternized. Heteroalkyls can be substituted as defined above for alkyl groups.

The term “alkylthio” refers to an alkyl group, as defined above, having a sulfur radical attached thereto. In some embodiments, the “alkylthio” moiety is represented by one of —S-alkyl, —S-alkenyl, and —S-alkynyl. Representative alkylthio groups include methylthio, and ethylthio. The term “alkylthio” also encompasses cycloalkyl groups, alkene and cycloalkene groups, and alkyne groups. “Arylthio” refers to aryl or heteroaryl groups. Alkylthio groups can be substituted as defined above for alkyl groups.

The terms “alkenyl” and “alkynyl”, refer to unsaturated aliphatic groups analogous in length and possible substitution to the alkyls described above, but that contain at least one double or triple bond respectively.

The terms “alkoxyl” or “alkoxy” as used herein refers to an alkyl group, as defined above, having an oxygen radical attached thereto. Representative alkoxyl groups include methoxy, ethoxy, propyloxy, and tert-butoxy. An “ether” is two hydrocarbons covalently linked by an oxygen. Accordingly, the substituent of an alkyl that renders that alkyl an ether is or resembles an alkoxyl, such as can be represented by one of —O-alkyl, —O-alkenyl, and —O-alkynyl. Aroxy can be represented by —O-aryl or O-heteroaryl, wherein aryl and heteroaryl are as defined below. The alkoxy and aroxy groups can be substituted as described above for alkyl.

The terms “amine” and “amino” are art-recognized and refer to both unsubstituted and substituted amines, e.g., a moiety that can be represented by the general formula:

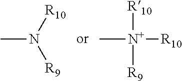

wherein R₉, R₁₀, and R′₁₀ each independently represent a hydrogen, an alkyl, an alkenyl, —(CH₂)ₘ—R₈ or R₉ and R₁₀ taken together with the N atom to which they are attached complete a heterocycle having from 4 to 8 atoms in the ring structure; R₈ represents an aryl, a cycloalkyl, a cycloalkenyl, a heterocycle or a polycycle; and m is zero or an integer in the range of 1 to 8. In some embodiments, only one of R₉ or R₁₀ can be a carbonyl, e.g., R₉, R₁₀ and the nitrogen together do not form an imide. In still other embodiments, the term “amine” does not encompass amides, e.g., wherein one of R₉ and R₁₀ represents a carbonyl. In additional embodiments, R₉ and R₁₀ (and optionally R′₁₀) each independently represent a hydrogen, an alkyl or cycloalkly, an alkenyl or cycloalkenyl, or alkynyl. Thus, the term “alkylamine” as used herein means an amine group, as defined above, having a substituted (as described above for alkyl) or unsubstituted alkyl attached thereto, i.e., at least one of R₉ and R₁₀ is an alkyl group.

The term “amido” is art-recognized as an amino-substituted carbonyl and includes a moiety that can be represented by the general formula:

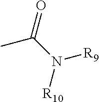

wherein R₉ and R₁₀ are as defined above.

“Aryl”, as used herein, refers to C₅-C₁₀-membered aromatic, heterocyclic, fused aromatic, fused heterocyclic, biaromatic, or bihetereocyclic ring systems. Broadly defined, “aryl”, as used herein, includes 5-, 6-, 7-, 8-, 9-, and 10-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics”. The aromatic ring can be substituted at one or more ring positions with one or more substituents including, but not limited to, halogen, azide, alkyl, aralkyl, alkenyl, alkynyl, cycloalkyl, hydroxyl, alkoxyl, amino (or quaternized amino), nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN; and combinations thereof.

The term “aryl” also includes polycyclic ring systems having two or more cyclic rings in which two or more carbons are common to two adjoining rings (i.e., “fused rings”) wherein at least one of the rings is aromatic, e.g., the other cyclic ring or rings can be cycloalkyls, cycloalkenyls, cycloalkynyls, aryls and/or heterocycles. Examples of heterocyclic rings include, but are not limited to, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH carbazolyl, carbolinyl, chromanyl, chromenyl, cinnolinyl, decahydroquinolinyl, 2H,6H-1,5,2-dithiazinyl, dihydrofuro[2,3 b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl. One or more of the rings can be substituted as defined above for “aryl”.

The term “aralkyl”, as used herein, refers to an alkyl group substituted with an aryl group (e.g., an aromatic or heteroaromatic group).

The term “carbocycle”, as used herein, refers to an aromatic or non-aromatic ring in which each atom of the ring is carbon.

“Heterocycle” or “heterocyclic”, as used herein, refers to a cyclic radical attached via a ring carbon or nitrogen of a monocyclic or bicyclic ring containing 3-10 ring atoms, and preferably from 5-6 ring atoms, consisting of carbon and one to four heteroatoms each selected from the group consisting of non-peroxide oxygen, sulfur, and N(Y) wherein Y is absent or is H, O, (C₁-C₁₀) alkyl, phenyl or benzyl, and optionally containing 1-3 double bonds and optionally substituted with one or more substituents. Examples of heterocyclic ring include, but are not limited to, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3 -b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxepanyl, oxetanyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydropyranyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl. Heterocyclic groups can optionally be substituted with one or more substituents at one or more positions as defined above for alkyl and aryl, for example, halogen, alkyl, aralkyl, alkenyl, alkynyl, cycloalkyl, hydroxyl, amino, nitro, sulfhydryl, imino, amido, phosphate, phosphonate, phosphinate, carbonyl, carboxyl, silyl, ether, alkylthio, sulfonyl, ketone, aldehyde, ester, a heterocyclyl, an aromatic or heteroaromatic moiety, —CF3, and —CN.

The term “carbonyl” is art-recognized and includes such moieties as can be represented by the general formula:

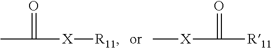

wherein X is a bond or represents an oxygen or a sulfur, and Ru represents a hydrogen, an alkyl, a cycloalkyl, an alkenyl, an cycloalkenyl, or an alkynyl, R′₁₁ represents a hydrogen, an alkyl, a cycloalkyl, an alkenyl, an cycloalkenyl, or an alkynyl. Where X is an oxygen and R₁₁ or R′₁₁ is not hydrogen, the formula represents an “ester”. Where X is an oxygen and R₁₁ is as defined above, the moiety is referred to herein as a carboxyl group, and particularly when R₁₁ is a hydrogen, the formula represents a “carboxylic acid”. Where X is an oxygen and R′₁₁ is hydrogen, the formula represents a “formate”. In general, where the oxygen atom of the above formula is replaced by sulfur, the formula represents a “thiocarbonyl” group. Where X is a sulfur and R₁₁ or R′₁₁ is not hydrogen, the formula represents a “thioester.” Where X is a sulfur and R₁₁ is hydrogen, the formula represents a “thiocarboxylic acid.” Where X is a sulfur and R′₁₁ is hydrogen, the formula represents a “thioformate.” On the other hand, where X is a bond, and R₁₁ is not hydrogen, the above formula represents a “ketone” group. Where X is a bond, and R₁₁ is hydrogen, the above formula represents an “aldehyde” group.

The term “monoester” as used herein refers to an analogue of a dicarboxylic acid wherein one of the carboxylic acids is functionalized as an ester and the other carboxylic acid is a free carboxylic acid or salt of a carboxylic acid. Examples of monoesters include, but are not limited to, to monoesters of succinic acid, glutaric acid, adipic acid, suberic acid, sebacic acid, azelaic acid, oxalic and maleic acid.

The term “heteroatom” as used herein means an atom of any element other than carbon or hydrogen. Examples of heteroatoms are boron, nitrogen, oxygen, phosphorus, sulfur and selenium. Other heteroatoms include silicon and arsenic.

As used herein, the term “nitro” means —NO₂; the term “halogen” designates —F, —Cl, —Br or —I; the term “sulfhydryl” means —SH; the term “hydroxyl” means —OH; and the term “sulfonyl” means —SO₂—.

The term “substituted” as used herein, refers to all permissible substituents of the compounds described herein. In the broadest sense, the permissible substituents include acyclic and cyclic, branched and unbranched, carbocyclic and heterocyclic, aromatic and nonaromatic substituents of organic compounds. Illustrative substituents include, but are not limited to, halogens, hydroxyl groups, or any other organic groupings containing any number of carbon atoms, preferably 1-14 carbon atoms, and optionally include one or more heteroatoms such as oxygen, sulfur, or nitrogen grouping in linear, branched, or cyclic structural formats. Representative substituents include alkyl, substituted alkyl, alkenyl, substituted alkenyl, alkynyl, substituted alkynyl, phenyl, substituted phenyl, aryl, substituted aryl, heteroaryl, substituted heteroaryl, halo, hydroxyl, alkoxy, substituted alkoxy, phenoxy, substituted phenoxy, aroxy, substituted aroxy, alkylthio, substituted alkylthio, phenylthio, substituted phenylthio, arylthio, substituted arylthio, cyano, isocyano, substituted isocyano, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C₃-C₂₀ cyclic, substituted C₃-C₂₀ cyclic, heterocyclic, substituted heterocyclic, aminoacid, peptide, and polypeptide groups.

Heteroatoms such as nitrogen may have hydrogen substituents and/or any permissible substituents of organic compounds described herein which satisfy the valences of the heteroatoms. It is understood that “substitution” or “substituted” includes the implicit proviso that such substitution is in accordance with permitted valence of the substituted atom and the substituent, and that the substitution results in a stable compound, i.e. a compound that does not spontaneously undergo transformation such as by rearrangement, cyclization, elimination, etc.

In a broad aspect, the permissible substituents include acyclic and cyclic, branched and unbranched, carbocyclic and heterocyclic, aromatic and nonaromatic substituents of organic compounds. Illustrative substituents include, for example, those described herein. The permissible substituents can be one or more and the same or different for appropriate organic compounds. The heteroatoms such as nitrogen may have hydrogen substituents and/or any permissible substituents of organic compounds described herein which satisfy the valencies of the heteroatoms.

In various embodiments, the substituent is selected from alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide, and thioketone, each of which optionally is substituted with one or more suitable substituents. In some embodiments, the substituent is selected from alkoxy, aryloxy, alkyl, alkenyl, alkynyl, amide, amino, aryl, arylalkyl, carbamate, carboxy, cycloalkyl, ester, ether, formyl, haloalkyl, heteroaryl, heterocyclyl, ketone, phosphate, sulfide, sulfinyl, sulfonyl, sulfonic acid, sulfonamide, and thioketone, wherein each of the alkoxy, aryloxy, alkyl, alkenyl, alkynyl, amide, amino, aryl, arylalkyl, carbamate, carboxy, cycloalkyl, ester, ether, formyl, haloalkyl, heteroaryl, heterocyclyl, ketone, phosphate, sulfide, sulfinyl, sulfonyl, sulfonic acid, sulfonamide, and thioketone can be further substituted with one or more suitable substituents.

Examples of substituents include, but are not limited to, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, thioketone, ester, heterocyclyl, —CN, aryl, aryloxy, perhaloalkoxy, aralkoxy, heteroaryl, heteroaryloxy, heteroarylalkyl, heteroaralkoxy, azido, alkylthio, oxo, acylalkyl, carboxy esters, carboxamido, acyloxy, aminoalkyl, alkylaminoaryl, alkylaryl, alkylaminoalkyl, alkoxyaryl, arylamino, aralkylamino, alkyl sulfonyl, carboxamidoalkylaryl, carboxamidoaryl, hydroxyalkyl, haloalkyl, alkylaminoalkylcarboxy, aminocarboxamidoalkyl, cyano, alkoxyalkyl, perhaloalkyl, arylalkyloxyalkyl, and the like. In some embodiments, the substituent is selected from cyano, halogen, hydroxyl, and nitro.

The term “copolymer” as used herein, generally refers to a single polymeric material that is comprised of two or more different monomers. The copolymer can be of any form, such as random, block, graft, etc. The copolymers can have any end-group, including capped or acid end groups.

The term “mean particle size”, as used herein, generally refers to the statistical mean particle size (diameter) of the particles in the composition. The diameter of an essentially spherical particle may be referred to as the physical or hydrodynamic diameter. The diameter of a non-spherical particle may refer preferentially to the hydrodynamic diameter. As used herein, the diameter of a non-spherical particle may refer to the largest linear distance between two points on the surface of the particle. Mean particle size can be measured using methods known in the art, such as dynamic light scattering. Two populations can be said to have a “substantially equivalent mean particle size” when the statistical mean particle size of the first population of nanoparticles is within 20% of the statistical mean particle size of the second population of nanoparticles; more preferably within 15%, most preferably within 10%.

The terms “monodisperse” and “homogeneous size distribution”, as used interchangeably herein, describe a population of particles, microparticles, or nanoparticles all having the same or nearly the same size. As used herein, a monodisperse distribution refers to particle distributions in which 90% of the distribution lies within 5% of the mean particle size.

The terms “polypeptide,” “peptide” and “protein” generally refer to a polymer of amino acid residues. As used herein, the term also applies to amino acid polymers in which one or more amino acids are chemical analogues or modified derivatives of corresponding naturally-occurring amino acids. The term “protein”, as generally used herein, refers to a polymer of amino acids linked to each other by peptide bonds to form a polypeptide for which the chain length is sufficient to produce tertiary and/or quaternary structure. The term “protein” excludes small peptides by definition, the small peptides lacking the requisite higher-order structure necessary to be considered a protein.

The terms “nucleic acid,” “polynucleotide,” and “oligonucleotide” are used interchangeably to refer to a deoxyribonucleotide or ribonucleotide polymer, in linear or circular conformation, and in either single- or double-stranded form. These terms are not to be construed as limiting with respect to the length of a polymer. The terms can encompass known analogues of natural nucleotides, as well as nucleotides that are modified in the base, sugar and/or phosphate moieties (e.g., phosphorothioate backbones). In general and unless otherwise specified, an analogue of a particular nucleotide has the same base-pairing specificity; i.e., an analogue of A will base-pair with T. The term “nucleic acid” is a term of art that refers to a string of at least two base-sugar-phosphate monomeric units. Nucleotides are the monomeric units of nucleic acid polymers. The term includes deoxyribonucleic acid (DNA) and ribonucleic acid (RNA) in the form of a messenger RNA, antisense, plasmid DNA, parts of a plasmid DNA or genetic material derived from a virus. Antisense is a polynucleotide that interferes with the function of DNA and/or RNA. The term nucleic acids refers to a string of at least two base-sugar-phosphate combinations. Natural nucleic acids have a phosphate backbone, artificial nucleic acids may contain other types of backbones, but contain the same bases. The term also includes PNAs (peptide nucleic acids), phosphorothioates, and other variants of the phosphate backbone of native nucleic acids.

A “functional fragment” of a protein, polypeptide or nucleic acid is a protein, polypeptide or nucleic acid whose sequence is not identical to the full-length protein, polypeptide or nucleic acid, yet retains at least one function as the full-length protein, polypeptide or nucleic acid. A functional fragment can possess more, fewer, or the same number of residues as the corresponding native molecule, and/or can contain one or more amino acid or nucleotide substitutions. Methods for determining the function of a nucleic acid (e.g., coding function, ability to hybridize to another nucleic acid) are well-known in the art. Similarly, methods for determining protein function are well-known. For example, the DNA binding function of a polypeptide can be determined, for example, by filter-binding, electrophoretic mobility shift, or immunoprecipitation assays. DNA cleavage can be assayed by gel electrophoresis. The ability of a protein to interact with another protein can be determined, for example, by co-immunoprecipitation, two-hybrid assays or complementation, e.g., genetic or biochemical. See, for example, Fields et al. (1989) Nature 340:245-246; U.S. Pat. No. 5,585,245 and PCT WO 98/44350.

As used herein, the term “linker” refers to a carbon chain that can contain heteroatoms (e.g., nitrogen, oxygen, sulfur, etc.) and which may be 1, 2, 3, 4, 5, 6, 7, 8, 9, 10, 11, 12, 13, 14, 15,16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 atoms long. Linkers may be substituted with various substituents including, but not limited to, hydrogen atoms, alkyl, alkenyl, alkynl, amino, alkylamino, dialkylamino, trialkylamino, hydroxyl, alkoxy, halogen, aryl, heterocyclic, aromatic heterocyclic, cyano, amide, carbamoyl, carboxylic acid, ester, thioether, alkylthioether, thiol, and ureido groups. Those of skill in the art will recognize that each of these groups may in turn be substituted. Examples of linkers include, but are not limited to, pH-sensitive linkers, protease cleavable peptide linkers, nuclease sensitive nucleic acid linkers, lipase sensitive lipid linkers, glycosidase sensitive carbohydrate linkers, hypoxia sensitive linkers, photo-cleavable linkers, heat-labile linkers, enzyme cleavable linkers (e.g., esterase cleavable linker), ultrasound-sensitive linkers, and x-ray cleavable linkers.

The term “pharmaceutically acceptable counter ion” refers to a pharmaceutically acceptable anion or cation. In various embodiments, the pharmaceutically acceptable counter ion is a pharmaceutically acceptable ion. For example, the pharmaceutically acceptable counter ion is selected from citrate, matate, acetate, oxalate, chloride, bromide, iodide, nitrate, sulfate, bisulfate, phosphate, acid phosphate, isonicotinate, acetate, lactate, salicylate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)). In some embodiments, the pharmaceutically acceptable counter ion is selected from chloride, bromide, iodide, nitrate, sulfate, bisulfate, phosphate, acid phosphate, citrate, malate, acetate, oxalate, acetate, and lactate. In particular embodiments, the pharmaceutically acceptable counter ion is selected from chloride, bromide, iodide, nitrate, sulfate, bisulfate, and phosphate.

The term “pharmaceutically acceptable salt(s)” refers to salts of acidic or basic groups that may be present in compounds used in the present compositions. Compounds included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including but not limited to sulfate, citrate, matate, acetate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds included in the present compositions that include an amino moiety may form pharmaceutically acceptable salts with various amino acids, in addition to the acids mentioned above. Compounds included in the present compositions, that are acidic in nature are capable of forming base salts with various pharmacologically acceptable cations. Examples of such salts include alkali metal or alkaline earth metal salts and, particularly, calcium, magnesium, sodium, lithium, zinc, potassium, and iron salts.

If the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare non-toxic pharmaceutically acceptable addition salts.

A pharmaceutically acceptable salt can be derived from an acid selected from 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, ascorbic acid, aspartic acid, benzenesulfonic acid, benzoic acid, camphoric acid, camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, glucoheptonic acid, gluconic acid, glucuronic acid, glutamic acid, glutaric acid, glycerophosphoric acid, glycolic acid, hippuric acid, hydrobromic acid, hydrochloric acid, isethionic, isobutyric acid, lactic acid, lactobionic acid, lauric acid, maleic acid, malic acid, malonic acid, mandelic acid, methanesulfonic acid, mucic, naphthalene-1,5-disulfonic acid, naphthalene-2-sulfonic acid, nicotinic acid, nitric acid, oleic acid, oxalic acid, palmitic acid, pamoic acid, pantothenic, phosphoric acid, proprionic acid, pyroglutamic acid, salicylic acid, sebacic acid, stearic acid, succinic acid, sulfuric acid, tartaric acid, thiocyanic acid, toluenesulfonic acid, trifluoroacetic, and undecylenic acid.

The term “bioavailable” is art-recognized and refers to a form of the subject invention that allows for it, or a portion of the amount administered, to be absorbed by, incorporated to, or otherwise physiologically available to a subject or patient to whom it is administered.

### II. Conjugates

Conjugates include an active agent or prodrug thereof attached to a targeting moiety by a linker. The conjugates can be a conjugate between a single active agent and a single targeting moiety, e.g. a conjugate having the structure X—Y—Z where X is the targeting moiety, Y is the linker, and Z is the active agent.

In some embodiments the conjugate contains more than one targeting moiety, more than one linker, more than one active agent, or any combination thereof. The conjugate can have any number of targeting moieties, linkers, and active agents. The conjugate can have the structure X—Y—Z—Y—X, (X—Y—Z—Y)ₙ—Z, X—(Y—Z)ₙ, X—Y—Zₙ, (X—Y—Z)ₙ, (X—Y—Z—Y)ₙ—Z where X is a targeting moiety, Y is a linker, Z is an active agent, and n is an integer between 1 and 50, between 2 and 20, more preferably between 1 and 5. Each occurrence of X, Y, and Z can be the same or different, e.g. the conjugate can contain more than one type of targeting moiety, more than one type of linker, and/or more than one type of active agent.

The conjugate can contain more than one targeting moiety attached to a single active agent. For example, the conjugate can include an active agent with multiple targeting moieties each attached via a different linker. The conjugate can have the structure X—Y—Z—Y—X where each X is a targeting moiety that may be the same or different, each Y is a linker that may be the same or different, and Z is the active agent.

The conjugate can contain more than one active agent attached to a single targeting moiety. For example the conjugate can include a targeting moiety with multiple active agents each attached via a different linker. The conjugate can have the structure Z—Y—X—Y—Z where X is the targeting moiety, each Y is a linker that may be the same or different, and each Z is an active agent that may be the same or different.

### A. Active Agents

The conjugate contains at least one active agent. The conjugate can contain more than one active agent, that can be the same or different. The active agent can be a therapeutic, prophylactic, diagnostic, or nutritional agent. A variety of active agents are known in the art and may be used in the conjugates. The active agent can be a protein or peptide, small molecule, nucleic acid or nucleic acid molecule, lipid, sugar, glycolipid, glycoprotein, lipoprotein, or combination thereof. In some embodiments, the active agent is an antigen or adjuvant, radioactive or imaging agent (e.g., a fluorescent moiety) or polynucleotide. In some embodiments the active agent is an organometallic compound.

### Anti-infective Agents

The active agent can be an anti-infective agent. Certain therapeutic agents are capable of preventing the establishment or growth (systemic or local) of a tumor or infection. Examples include boron-containing compounds (e.g., carborane), chemotherapeutic nucleotides, drugs (e.g., antibiotics, antivirals, antifungals), enediynes (e.g., calicheamicins, esperamicins, dynemicin, neocarzinostatin chromophore, and kedarcidin chromophore), heavy metal complexes (e.g., cisplatin), hormone antagonists (e.g., tamoxifen), non-specific (non-antibody) proteins (e.g., sugar oligomers), oligonucleotides (e.g., antisense oligonucleotides that bind to a target nucleic acid sequence (e.g., mRNA sequence)), peptides, photodynamic agents (e.g., rhodamine 123), radionuclides (e.g., I-131, Re-186, Re-188, Y-90, Bi-212, At-211, Sr-89, Ho-166, Sm-153, Cu-67 and Cu-64), toxins (e.g., ricin), and transcription-based pharmaceuticals. The therapeutic agent can be a small molecule, radionuclide, toxin, hormone antagonist, heavy metal complex, oligonucleotide, chemotherapeutic nucleotide, peptide, non-specific (non-antibody) protein, a boron compound or an enediyne.

The active agent can treat or prevent the establishment or growth of a bacterial infection. The therapeutic agent can be an antibiotic, radionuclide or oligonucleotide. The active agent can treat or prevent the establishment or growth of a viral infection, e.g. the active agent can be an antiviral compound, radionuclide or oligonucleotide. The active agent can treat or prevent the establishment or growth of a fungal infection, e.g. the active agent can be an antifungal compound, radionuclide or oligonucleotide.

### Anti-cancer Agents

The active agent can be a cancer therapeutic. The cancer therapeutics may include death receptor agonists such as the TNF-related apoptosis-inducing ligand (TRAIL) or Fas ligand or any ligand or antibody that binds or activates a death receptor or otherwise induces apoptosis. Suitable death receptors include, but are not limited to, TNFR1, Fas, DR3, DR4, DR5, DR6, LTβR and combinations thereof.

Conventional cancer therapeutics such as chemotherapeutic agents, cytokines, chemokines, and radiation therapy can be used as active agents. The majority of chemotherapeutic drugs can be divided in to: alkylating agents, antimetabolites, anthracyclines, plant alkaloids, topoisomerase inhibitors, and other antitumour agents. All of these drugs affect cell division or DNA synthesis and function in some way. Additional therapeutics that can be used as active agents include monoclonal antibodies and the tyrosine kinase inhibitors e.g. imatinib mesylate (GLEEVEC® or GLIVEC®), which directly targets a molecular abnormality in certain types of cancer (chronic myelogenous leukemia, gastrointestinal stromal tumors).

Representative chemotherapeutic agents include, but are not limited to cisplatin, carboplatin, oxaliplatin, mechlorethamine, cyclophosphamide, chlorambucil, vincristine, vinblastine, vinorelbine, vindesine, taxol and derivatives thereof, irinotecan, topotecan, amsacrine, etoposide, etoposide phosphate, teniposide, epipodophyllotoxins, trastuzumab (HERCEPTIN®), cetuximab, and rituximab (RITUXAN® or MABTHERA®), bevacizumab (AVASTIN®), and combinations thereof. Any of these may be used as an active agent in a conjugate.

The active agent can be 20-epi-1,25 dihydroxyvitamin D3, 4-ipomeanol, 5-ethynyluracil, 9-dihydrotaxol, abiraterone, acivicin, aclarubicin, acodazole hydrochloride, acronine, acylfulvene, adecypenol, adozelesin, aldesleukin, all-tk antagonists, altretamine, ambamustine, ambomycin, ametantrone acetate, amidox, amifostine, aminoglutethimide, aminolevulinic acid, amrubicin, amsacrine, anagrelide, anastrozole, andrographolide, angiogenesis inhibitors, antagonist D, antagonist G, antarelix, anthramycin, anti-dorsalizing morphogenetic protein-1, antiestrogen, antineoplaston, antisense oligonucleotides, aphidicolin glycinate, apoptosis gene modulators, apoptosis regulators, apurinic acid, ARA-CDP-DL-PTBA, arginine deaminase, asparaginase, asperlin, asulacrine, atamestane, atrimustine, axinastatin 1, axinastatin 2, axinastatin 3, azacitidine, azasetron, azatoxin, azatyrosine, azetepa, azotomycin, baccatin III derivatives, balanol, batimastat, benzochlorins, benzodepa, benzoylstaurosporine, beta lactam derivatives, beta-alethine, betaclamycin B, betulinic acid, BFGF inhibitor, bicalutamide, bisantrene, bisantrene hydrochloride, bisaziridinylspermine, bisnafide, bisnafide dimesylate, bistratene A, bizelesin, bleomycin, bleomycin sulfate, BRC/ABL antagonists, breflate, brequinar sodium, bropirimine, budotitane, busulfan, buthionine sulfoximine, cabazitaxel, cactinomycin, calcipotriol, calphostin C, calusterone, camptothecin, camptothecin derivatives, canarypox IL-2, capecitabine, caracemide, carbetimer, carboplatin, carboxamide-amino-triazole, carboxyamidotriazole, carest M3, carmustine, earn 700, cartilage derived inhibitor, carubicin hydrochloride, carzelesin, casein kinase inhibitors, castano spermine, cecropin B, cedefingol, cetrorelix, chlorambucil, chlorins, chloroquinoxaline sulfonamide, cicaprost, cirolemycin, cisplatin, cis-porphyrin, cladribine, clomifene analogs, clotrimazole, collismycin A, collismycin B, combretastatin A4, combretastatin analog, conagenin, crambescidin 816, crisnatol, crisnatol mesylate, cryptophycin 8, cryptophycin A derivatives, curacin A, cyclopentanthraquinones, cyclophosphamide, cycloplatam, cypemycin, cytarabine, cytarabine ocfosfate, cytolytic factor, cytostatin, dacarbazine, dacliximab, dactinomycin, daunorubicin hydrochloride, decitabine, dehydrodidemnin B, deslorelin, dexifosfamide, dexormaplatin, dexrazoxane, dexverapamil, dezaguanine, dezaguanine mesylate, diaziquone, didemnin B, didox, diethylnorspermine, dihydro-5-azacytidine, dioxamycin, diphenyl spiromustine, docetaxel, docosanol, dolasetron, doxifluridine, doxorubicin, doxorubicin hydrochloride, droloxifene, droloxifene citrate, dromostanolone propionate, dronabinol, duazomycin, duocarmycin SA, ebselen, ecomustine, edatrexate, edelfosine, edrecolomab, eflornithine, eflornithine hydrochloride, elemene, elsamitrucin, emitefur, enloplatin, enpromate, epipropidine, epirubicin, epirubicin hydrochloride, epristeride, erbulozole, erythrocyte gene therapy vector system, esorubicin hydrochloride, estramustine, estramustine analog, estramustine phosphate sodium, estrogen agonists, estrogen antagonists, etanidazole, etoposide, etoposide phosphate, etoprine, exemestane, fadrozole, fadrozole hydrochloride, fazarabine, fenretinide, filgrastim, finasteride, flavopiridol, flezelastine, floxuridine, fluasterone, fludarabine, fludarabine phosphate, fluorodaunorunicin hydrochloride, fluorouracil, flurocitabine, forfenimex, formestane, fosquidone, fostriecin, fostriecin sodium, fotemustine, gadolinium texaphyrin, gallium nitrate, galocitabine, ganirelix, gelatinase inhibitors, gemcitabine, gemcitabine hydrochloride, glutathione inhibitors, hepsulfam, heregulin, hexamethylene bisacetamide, hydroxyurea, hypericin, ibandronic acid, idarubicin, idarubicin hydrochloride, idoxifene, idramantone, ifosfamide, ilmofosine, ilomastat, imidazoacridones, imiquimod, immunostimulant peptides, insulin-like growth factor-1 receptor inhibitor, interferon agonists, interferon alpha-2A, interferon alpha-2B, interferon alpha-N1, interferon alpha-N3, interferon beta-IA, interferon gamma-IB, interferons, interleukins, iobenguane, iododoxorubicin, iproplatin, irinotecan, irinotecan hydrochloride, iroplact, irsogladine, isobengazole, isohomohalicondrin B, itasetron, jasplakinolide, kahalalide F, lamellarin-N triacetate, lanreotide, larotaxel, lanreotide acetate, leinamycin, lenograstim, lentinan sulfate, leptolstatin, letrozole, leukemia inhibiting factor, leukocyte alpha interferon, leuprolide acetate, leuprolide/estrogen/progesterone, leuprorelin, levamisole, liarozole, liarozole hydrochloride, linear polyamine analog, lipophilic disaccharide peptide, lipophilic platinum compounds, lissoclinamide 7, lobaplatin, lombricine, lometrexol, lometrexol sodium, lomustine, lonidamine, losoxantrone, losoxantrone hydrochloride, lovastatin, loxoribine, lurtotecan, lutetium texaphyrin, lysofylline, lytic peptides, maitansine, mannostatin A, marimastat, masoprocol, maspin, matrilysin inhibitors, matrix metalloproteinase inhibitors, maytansine, mechlorethamine hydrochloride, megestrol acetate, melengestrol acetate, melphalan, menogaril, merbarone, mercaptopurine, meterelin, methioninase, methotrexate, methotrexate sodium, metoclopramide, metoprine, meturedepa, microalgal protein kinase C inhibitors, MIF inhibitor, mifepristone, miltefosine, mirimostim, mismatched double stranded RNA, mitindomide, mitocarcin, mitocromin, mitogillin, mitoguazone, mitolactol, mitomalcin, mitomycin, mitomycin analogs, mitonafide, mitosper, mitotane, mitotoxin fibroblast growth factor-saporin, mitoxantrone, mitoxantrone hydrochloride, mofarotene, molgramostim, monoclonal antibody, human chorionic gonadotrophin, monophosphoryl lipid a/myobacterium cell wall SK, mopidamol, multiple drug resistance gene inhibitor, multiple tumor suppressor 1-based therapy, mustard anticancer agent, mycaperoxide B, mycobacterial cell wall extract, mycophenolic acid, myriaporone, n-acetyldinaline, nafarelin, nagrestip, naloxone/pentazocine, napavin, naphterpin, nartograstim, nedaplatin, nemorubicin, neridronic acid, neutral endopeptidase, nilutamide, nisamycin, nitric oxide modulators, nitroxide antioxidant, nitrullyn, nocodazole, nogalamycin, n-substituted benzamides, 06-benzylguanine, octreotide, okicenone, oligonucleotides, onapristone, ondansetron, oracin, oral cytokine inducer, ormaplatin, osaterone, oxaliplatin, oxaunomycin, oxisuran, paclitaxel, paclitaxel analogs, paclitaxel derivatives, palauamine, palmitoylrhizoxin, pamidronic acid, panaxytriol, panomifene, parabactin, pazelliptine, pegaspargase, peldesine, peliomycin, pentamustine, pentosan polysulfate sodium, pentostatin, pentrozole, peplomycin sulfate, perflubron, perfosfamide, perillyl alcohol, phenazinomycin, phenylacetate, phosphatase inhibitors, picibanil, pilocarpine hydrochloride, pipobroman, piposulfan, pirarubicin, piritrexim, piroxantrone hydrochloride, placetin A, placetin B, plasminogen activator inhibitor, platinum(IV) complexes, platinum compounds, platinum-triamine complex, plicamycin, plomestane, porfimer sodium, porfiromycin, prednimustine, procarbazine hydrochloride, propyl bis-acridone, prostaglandin J2, prostatic carcinoma antiandrogen, proteasome inhibitors, protein A-based immune modulator, protein kinase C inhibitor, protein tyrosine phosphatase inhibitors, purine nucleoside phosphorylase inhibitors, puromycin, puromycin hydrochloride, purpurins, pyrazofurin, pyrazoloacridine, pyridoxylated hemoglobin polyoxy ethylene conjugate, RAF antagonists, raltitrexed, ramosetron, RAS farnesyl protein transferase inhibitors, RAS inhibitors, RAS-GAP inhibitor, retelliptine demethylated, rhenium RE 186 etidronate, rhizoxin, riboprine, ribozymes, RH retinamide, RNAi, rogletimide, rohitukine, romurtide, roquinimex, rubiginone B1, ruboxyl, safingol, safingol hydrochloride, saintopin, sarcnu, sarcophytol A, sargramostim, SDI 1 mimetics, semustine, senescence derived inhibitor 1 , sense oligonucleotides, siRNA, signal transduction inhibitors, signal transduction modulators, simtrazene, single chain antigen binding protein, sizofiran, sobuzoxane, sodium borocaptate, sodium phenylacetate, solverol, somatomedin binding protein, sonermin, sparfosate sodium, sparfosic acid, sparsomycin, spicamycin D, spirogermanium hydrochloride, spiromustine, spiroplatin, splenopentin, spongistatin 1, squalamine, stem cell inhibitor, stem-cell division inhibitors, stipiamide, streptonigrin, streptozocin, stromelysin inhibitors, sulfinosine, sulofenur, superactive vasoactive intestinal peptide antagonist, suradista, suramin, swainsonine, synthetic glycosaminoglycans, talisomycin, tallimustine, tamoxifen methiodide, tauromustine, tazarotene, tecogalan sodium, tegafur, tellurapyrylium, telomerase inhibitors, teloxantrone hydrochloride, temoporfin, temozolomide, teniposide, teroxirone, testolactone, tetrachlorodecaoxide, tetrazomine, thaliblastine, thalidomide, thiamiprine, thiocoraline, thioguanine, thiotepa, thrombopoietin, thrombopoietin mimetic, thymalfasin, thymopoietin receptor agonist, thymotrinan, thyroid stimulating hormone, tiazofurin, tin ethyl etiopurpurin, tirapazamine, titanocene dichloride, topotecan hydrochloride, topsentin, toremifene, toremifene citrate, totipotent stem cell factor, translation inhibitors, trestolone acetate, tretinoin, triacetyluridine, triciribine, triciribine phosphate, trimetrexate, trimetrexate glucuronate, triptorelin, tropisetron, tubulozole hydrochloride, turosteride, tyrosine kinase inhibitors, tyrphostins, UBC inhibitors, ubenimex, uracil mustard, uredepa, urogenital sinus-derived growth inhibitory factor, urokinase receptor antagonists, vapreotide, variolin B, velaresol, veramine, verdins, verteporfin, vinblastine sulfate, vincristine sulfate, vindesine, vindesine sulfate, vinepidine sulfate, vinglycinate sulfate, vinleurosine sulfate, vinorelbine, vinorelbine tartrate, vinrosidine sulfate, vinxaltine, vinzolidine sulfate, vitaxin, vorozole, zanoterone, zeniplatin, zilascorb, zinostatin, zinostatin stimalamer, or zorubicin hydrochloride.

In preferred embodiments the active agent is cabazitaxel, or an analogue, derivative, prodrug, or pharmaceutically acceptable salt thereof.

The active agent can be an inorganic or organometallic compound containing one or more metal centers, preferably one metal center. The active agent can be a platinum compound (as described herein), a ruthenium compound (e.g., trans-[RuCl2 (DMSO)₄], or trans-[RuCl₄(imidazole)₂, etc.), cobalt compounds, copper compounds, iron compounds, etc.

In some embodiments, the active agent is a platinum complex in the 4+ oxidative state (Pt(IV) complexes). The active agent can be a compound of Formula I:

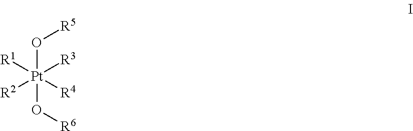

or a pharmaceutically acceptable salt thereof, where two of R¹, R², R³, and R⁴ are each independently a halide, carboxylate, sulfonate, sulfate, phosphate, or nitrate; the remaining two or R¹, R², R³, and R⁴ are each independently ammonia or an amine; and R5 and R6 are each independently hydrogen, R⁷, or

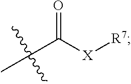

where X is absent, C(R⁸)₂, O, S, or NR⁸, and R⁷ and R⁸ are independently at each occurrence selected from hydrogen, alkyl, alkenyl, alkynyl, cycloalkyl, heterocyclyl, aryl, and heteroaryl, wherein each of the alkyl, alkenyl, alkynyl, cycloalkyl, heterocyclyl, aryl, and heteroaryl groups optionally is substituted with one or more groups, each independently selected from halogen, cyano, nitro, hydroxyl, ester, ether, alkoxy, aryloxy, amino, amide, carbamate, alkyl, alkenyl, alkynyl, aryl, arylalkyl, cycloalkyl, heteroaryl, heterocyclyl, oxo, phosphono, phosphate, sulfide, sulfinyl, sulfino, sulfonyl, sulfo, and sulfonamide, wherein each of the ester, ether, alkoxy, aryloxy, amino, amide, carbamate, alkyl, alkenyl, alkynyl, aryl, arylalkyl, cycloalkyl, heteroaryl, heterocyclyl, phosphono, phosphate, sulfide, sulfinyl, sulfino, sulfonyl, sulfo, and sulfonamide is optionally substituted with one or more suitable substituents.

In some embodiments, the compound is not ethacraplatin, cis,cis,trans-[Pt(NH₃)₂Cl₂(OH)₂], cis,cis,trans-[Pt(NH₂(isopropyl))₂Cl₂(OH)₂], cis, cis, trans-[Pt(NH₃)₂Cl₂(O₂C(CH₂)₄CH₃)₂], cis,cis,trans-[Pt(NH₃)₂Cl₂(O₂C(CH₂)₂CO₂H)₂],¹ cis,cis,trans-[Pt(NH₃)₂Cl₂(O₂CCF₃)₂], (cis,cis,trans-[Pt(NH₃)₂Cl₂(O₂CCHCl₂)₂], cis,cis,trans-[Pt(NH₃)₂Cl₂(O₂CCH₃)₂], cis, cis, trans [PtNH₃(NH₂(isopropyl))Cl₂(O₂CCH₃)₂], cis, cis,trans-[PtNH₃(NH₂(cyclohexyl))Cl₂(O₂CCH₃)₂], cis,cis,trans[PtNH₃(NH₂(adamantyl))Cl₂(O₂CCH₃)₂], cis,cis,trans-[PtNH₃(NH₂(cyclohexyl))Cl₂(O₂C(CH₂)₅CH₃)₂], cis,cis,trans-[Pt(NH₃)₂Cl₂(O₂CNHC(CH₃)₃)₂], cis, cis, trans-[Pt(NH₃)₂Cl₂(O₂CNH(cyclopentyl))₂], or cis,cis,trans-[Pt(NH₃)2Cl₂(O₂CNH(cyclohexyl))₂].

In some embodiments, at least one of R¹, R², R³, and R⁴ is a halide. For example, at least one of R¹, R², R³, and R⁴ is Cl. In some embodiments, two of R¹, R², R³, and R⁴ each is a halide. In some embodiments, two of R¹, R², R³, and R⁴ each is Cl.

In some embodiments, at least one of R¹, R², R³, and R⁴ is —O(C═O)R^{a}, and R^{a} is hydrogen, alkyl, aryl, arylalkyl, or cycloalkyl, wherein each of the alkyl, aryl, arylalkyl, and cycloalkyl is optionally substituted with one or more suitable substituents. For example, at least one of R¹, R², R³, and R⁴ can be formyl, acetate, propionate, butyrate, benzoate, sulfonate (including tosylate), phosphate, or sulfate.

In some embodiments, two of R¹, R², R³, and R⁴ each is —O(C═O)R^{a}, and R^{a} is hydrogen, alkyl, aryl, arylalkyl, or cycloalkyl, wherein each of the alkyl, aryl, arylalkyl, and cycloalkyl is optionally substituted with one or more suitable substituents. In some embodiments, two of R¹, R², R³, and R⁴ each is formyl, acetate, propionate, butyrate, or benzoate. In some embodiments, two of R¹, R², R³, and R⁴ each is a sulfonate, phosphate, or sulfate. For example, two of R¹, R², R³, and R⁴ each can be tosylate.

In various embodiments, at least one of R¹, R², R³, and R⁴ is ammonia. In some embodiments, two of R¹, R², R³, and R⁴ each is ammonia.

In various embodiments, at least one of R¹, R², R³, and R⁴ is an amine. In some embodiments, two of R¹, R², R³, and R⁴ each is an amine.

In some embodiments the active agents have two ligands (e.g., R¹, R², R³, and R⁴) positioned in a cis configuration, i.e., the compound may be a cis isomer. However, it should be understood that compounds of the present teachings may also have two ligands (e.g., R¹, R², R³, and R⁴) positioned in a trans configuration, i.e., the compound may be a trans isomer. Those of ordinary skill in the art would understand the meaning of these terms.

The active agent can be a compound according to Formula Ia:

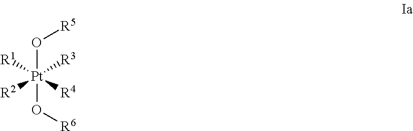

wherein R¹, R², R³, R⁴, R⁵, and R⁶ are as defined herein.

In some embodiments, at least one of R³ and R⁴ is a halide, hydroxyl, formyl, acetate, propionate, butyrate, benzoate, sulfonate (including tosylate), phosphate, or sulfate. In certain embodiments, at least one of R³ and R⁴ is a halide. In particular embodiments, both R³ and R⁴ are Cl. In certain embodiments, at least one of R³ and R⁴ is hydroxyl. In particular embodiments, both R³ and R⁴ are hydroxyl.

In some embodiments, at least one of R¹ and R² is ammonia. In some embodiments, at least one of R¹ and R² is an amine. For example, at least one of R¹ and R² is an alkylamine, alkenylamine, alkynylamine, arylamine, arylalkylamine, cycloalkylamine, heterocycloalkylamine, or heteroarylamine. In certain embodiments, one of R¹ and R² is methylamine, ethylamine, propylamine, isopropylamine, butylamine, isobutylamine, tertbutylamine, cyclopentylamine, cyclohexylamine, or adamantylamine. In certain embodiments, both R¹ and R² are ammonia.

In some embodiments, any two ligands (e.g., R¹, R², R³, and R⁴) may be joined together to form a bidentate or tridentate ligand, respectively. As will be known to those of ordinary skill in the art, a bidentate ligand, as used herein, when bound to a metal center, forms a metallacycle structure with the metal center, also known as a chelate ring. Bidentate ligands suitable for use in the present teachings include species that have at least two sites capable of binding to a metal center. For example, the bidentate ligand may comprise at least two heteroatoms that coordinate the metal center, or a heteroatom and an anionic carbon atom that coordinate the metal center.

Examples of bidentate ligands suitable for use in the present teachings include, but are not limited to, alkyl and aryl derivatives of moieties such as amines, phosphines, phosphites, phosphates, imines, oximes, ethers, alcohols, thiolates, thioethers, hybrids thereof, substituted derivatives thereof, aryl groups (e.g., bis-aryl, heteroaryl-substituted aryl), heteroaryl groups, and the like. Specific examples of bidentate ligands include ethylenediamine, 2,2′-bipyridine, acetylacetonate, oxalate, and the like. Other non-limiting examples of bidentate ligands include diimines, pyridylimines, diamines, imineamines, iminethioether, iminephosphines, bisoxazoline, bisphosphineimines, diphosphines, phosphineamine, salen and other alkoxy imine ligands, amidoamines, imidothioether fragments and alkoxyamide fragments, and combinations of the above ligands.

A tridentate ligand, as used herein, generally includes species which have at least three sites capable of binding to a metal center. For example, the tridentate ligand may comprise at least three heteroatoms that coordinate the metal center, or a combination of heteroatom(s) and anionic carbon atom(s) that coordinate the metal center. Non-limiting examples of tridentate ligands include 2,5-diiminopyridyl ligands, tripyridyl moieties, triimidazoyl moieties, tris pyrazoyl moieties, and combination of the above ligands.

In various embodiments, one of R⁵ and R⁶ is hydrogen. In various embodiments, at least one of R⁵ and R⁶ is R⁷. For example, R⁵ can be hydrogen and R⁶ can be R⁷ or R⁶ can be hydrogen and R⁵ can be R⁷. In some embodiments, both R⁵ and R⁶ are R⁷.

In some embodiments, at least one of R⁵ and R⁶ is

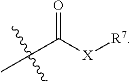

For example, R⁵ can be hydrogen and R⁶ can be

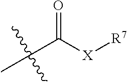

or R⁶ can be hydrogen and R⁵ can be

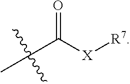

In some embodiments, both R⁵ and R⁶ are

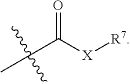

In some embodiments, X is absent.

In some embodiments, X is C(R⁸)₂, wherein R⁸ is as defined herein. In various embodiments, X is NR⁸, where R⁸ is as defined herein.

In some embodiments, R⁸ at each occurrence is hydrogen or alkyl, optionally substituted with one or more groups, each independently selected from halogen, cyano, nitro, ester, ether, alkoxy, aryloxy, amide, carbamate, alkenyl, alkynyl, aryl, arylalkyl, cycloalkyl, heteroaryl, heterocyclyl, and oxo, wherein each of the ester, ether, alkoxy, aryloxy, amide, carbamate, alkenyl, alkynyl, aryl, arylalkyl, cycloalkyl, heteroaryl, and heterocyclyl is optionally substituted with one or more suitable substituents. In some embodiments, R⁸ at least at one occurrence is hydrogen. In some embodiments, R⁸ at least at one occurrence is an optionally substituted alkyl. For example, R⁸ at least at one occurrence is an alkyl (e.g., methyl, ethyl, propyl, or isopropyl).

In particular embodiments, X is CH₂ or C(CH₃)₂. In particular embodiments, X is NH.

In some embodiments, R⁷ is alkyl or cycloalkyl. For example, R⁷ is alkyl optionally substituted with one or more groups each independently selected from halogen, hydroxyl, ester, alkoxy, aryloxy, amino, amide, aryl, arylalkyl, cycloalkyl, heteroaryl, and heterocyclyl, wherein each of ester, alkoxy, aryloxy, amino, amide, aryl, arylalkyl, cycloalkyl, heteroaryl, and heterocyclyl optionally is substituted with one or more suitable substituents. In some embodiments, R⁷ is alkyl optionally substituted with one or more groups each independently selected from halogen, hydroxyl, alkoxy, aryloxy, arylalkoxy, amino, amide, and aryl, wherein each of alkoxy, aryloxy, arylalkoxy, amino, amide, and aryl optionally is substituted with one or more substituents, each independently selected from one or more suitable substituents. In certain embodiments, R⁷ is alkyl optionally substituted with one or more groups each independently selected from F, Cl, phenyl, benzyloxy, t-butylphenyl, amino, and bistrifluoromethylphenyl. In particular embodiments, R⁷ is benzyl. In particular embodiments, R⁷ is butyl, tert-butyl, octyl, dodecanyl, 1,1,3,3,-tetramethylbutyl, 2-ethylhexyl, 2,2-dimethylpropyl, 2,2,3,3,4,4,4-heptafluorobutyl, aminomethyl, tert-butoxycarbonylaminomethyl, hydroxylcarbonylmethyl, diphenylmethyl, 4′-t-butylbenzyl, 2-benzyloxylethyl, or 3′,5′-ditrifluoromethylbenzyl.

In some embodiments, R⁷ is cycloalkyl. For example, R⁷ can be monocyclic, bicyclic, or bridged cyclic cycloalkyl having 3-14 ring carbons. In some embodiments, R⁷ is cycloalkyl optionally substituted with one or more groups each independently selected from halogen, hydroxyl, ester, alkoxy, aryloxy, amino, amide, alkyl, alkenyl, alkynyl, aryl, arylalkyl, cycloalkyl, heteroaryl, and heterocyclyl, wherein each of ester, alkoxy, aryloxy, amino, amide, alkyl, alkenyl, alkynyl, aryl, arylalkyl, cycloalkyl, heteroaryl, and heterocyclyl optionally is substituted with one or more suitable substituents. For example, R⁷ can be cycloalkyl optionally substituted with one or more groups each independently selected from halogen, hydroxyl, alkoxy, aryloxy, arylalkoxy, amino, amide, alkyl, alkenyl, and aryl, wherein each of alkoxy, aryloxy, arylalkoxy, amino, amide, alkyl, alkenyl, and aryl optionally is substituted with one or more substituents, each independently selected from one or more suitable substituents.

In certain embodiments, R⁷ is selected from cyclohexyl, cycloheptyl, cyclooctyl, cyclopentyl, cyclodecanyl, cycloundecanyl, cyclododecanyl, camphanyl, camphenyl, or adamantyl. In particular embodiments, R⁷ is cyclohexyl, cyclododecanyl, or adamantyl.

In some embodiments, R⁷ is at each occurrence is selected from aryl and heteroaryl, wherein each of the aryl and heteroaryl groups optionally is substituted with one or more groups, each independently selected from halogen, cyano, nitro, hydroxyl, ester, ether, alkoxy, aryloxy, amino, amide, carbamate, alkyl, alkenyl, alkynyl, aryl, arylalkyl, cycloalkyl, heteroaryl, heterocyclyl, phosphono, phosphate, sulfide, sulfinyl, sulfino, sulfonyl, sulfo, and sulfonamide, wherein each of the ester, ether, alkoxy, aryloxy, amino, amide, carbamate, alkyl, alkenyl, alkynyl, aryl, arylalkyl, cycloalkyl, heteroaryl, heterocyclyl, phosphono, phosphate, sulfide, sulfinyl, sulfino, sulfonyl, sulfo, and sulfonamide is optionally substituted with one or more suitable substituents. In some embodiments, R⁷ at each occurrence is aryl optionally substituted with one or more groups, each independently selected from halogen, cyano, nitro, hydroxyl, ester, ether, alkoxy, aryloxy, amino, amide, carbamate, alkyl, alkenyl, alkynyl, aryl, arylalkyl, cycloalkyl, heteroaryl, and heterocyclyl, wherein each of the ester, ether, alkoxy, aryloxy, amino, amide, carbamate, alkyl, alkenyl, alkynyl, aryl, arylalkyl, cycloalkyl, heteroaryl, and heterocyclyl is optionally substituted with one or more suitable substituents. For example, R⁷ is aryl optionally substituted with one or more groups, each independently selected from halogen, cyano, nitro, hydroxyl, eter, ether, alkoxy, aryloxy, amino, amide, alkyl, aryl, arylalkyl, cycloalkyl, heteroaryl, and heterocyclyl. In certain embodiments, R⁷ is pheny optionally substituted with one or more groups, each independently selected from halogen, cyano, nitro, hydroxyl, eter, ether, alkoxy, aryloxy, amino, amide, alkyl, aryl, arylalkyl, cycloalkyl, heteroaryl, and heterocyclyl. In particular embodiments, R⁷ is phenyl.

In various embodiments, R⁵ and R⁶ are different. For example, the compound of the present teachings can be selected from:

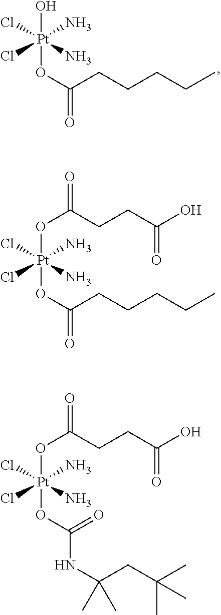

In various embodiments, R⁵ and R⁶ can be the same. For example, the compound of the present teachings can be selected from:

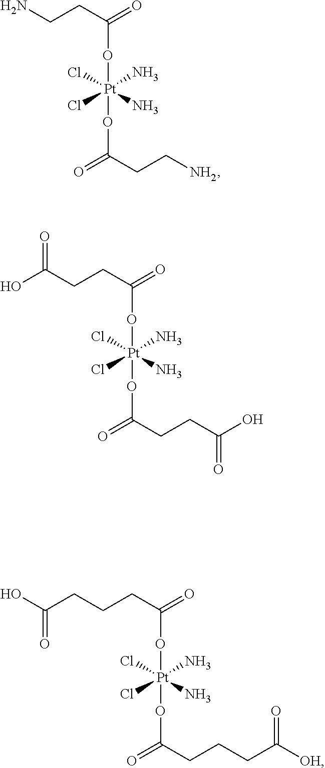

In certain embodiments, the active agent of the conjugate comprises a predetermined molar weight percentage from about 1% to 10%, or about 10% to about 20%, or about 20% to about 30%, or about 30% to 40%, or about 40% to 50%, or about 50% to 60%, or about 60% to 70%, or about 70% to 80%, or about 80% to 90%, or about 90% to 99% such that the sum of the molar weight percentages of the components of the conjugate is 100%. The amount of active agent(s) of the conjugate may also be expressed in terms of proportion to the targeting ligand(s). For example, the present teachings provide a ratio of active agent to ligand of about 10:1, 9:1, 8:1, 7:1, 6:1, 5:1, 4:1, 3:1, 2:1, 1:1, 1:2, 1:3, 1:4; 1:5, 1:6, 1:7, 1:8, 1:9, or 1:10.

### B. Targeting Moieties

The conjugates contain one or more targeting moieties and/or targeting ligands. Targeting ligands or moieties can be peptides, antibody mimetics, nucleic acids (e.g., aptamers), polypeptides (e.g., antibodies), glycoproteins, small molecules, carbohydrates, or lipids. The targeting moiety, X, can be a peptide such as somatostatin, octreotide, an EGFR-binding peptide or RGD-containing peptides, nucleic acid (e.g., aptamer), polypeptide (e.g., antibody or its fragment), glycoprotein, small molecule, carbohydrate, or lipid. The targeting moiety, X can be an aptamer being either RNA or DNA or an artificial nucleic acid; small molecules; carbohydrates such as mannose, galactose and arabinose; vitamins such as ascorbic acid, niacin, pantothenic acid, carnitine, inositol, pyridoxal, lipoic acid, folic acid (folate), riboflavin, biotin, vitamin B₁₂, vitamin A, E, and K; a protein or peptide that binds to a cell-surface receptor such as a receptor for thrombospondin, tumor necrosis factors (TNF), annexin V, interferons, cytokines, transferrin, GM-CSF (granulocyte-macrophage colony-stimulating factor), or growth factors such as vascular endothelial growth factor (VEGF), hepatocyte growth factor (HGF), (platelet-derived growth factor (PDGF), basic fibroblast growth factor (bFGF), and epidermal growth factor (EGF).

In some embodiments, the targeting moiety is an antibody mimetic such as a monobody, e.g., an ADNECTIN™ (Bristol-Myers Squibb, New York, N.Y.), an Affibody® (Affibody A B, Stockholm, Sweden), Affilin, nanofitin (affitin, such as those described in WO 2012/085861, an Anticalin™, an avimers (avidity multimers), a DARPin™, a Fynomer™, and a Kunitz domain peptide. In certain cases, such mimetics are artificial peptides or proteins with a molar mass of about 3 to 20 kDa. Nucleic acids and small molecules may be antibody mimetic.

In some embodiments, the targeting moiety is arginylglycylaspartic acid (RGD peptide), a tripeptide composed of L-arginine, glycine, and L-aspartic acid. The sequence is a common element in cellular recognition. Arginylglycylaspartic acid displays a strong affinity and selectivity to the alpha-V-beta-3 integrin found in tumor cells.

In another example, a targeting moiety can be an aptamer, which is generally an oligonucleotide (e.g., DNA, RNA, or an analog or derivative thereof) that binds to a particular target, such as a polypeptide. In some embodiments, the targeting moiety is a polypeptide (e.g., an antibody that can specifically bind a tumor marker). In certain embodiments, the targeting moiety is an antibody or a fragment thereof. In certain embodiments, the targeting moiety is an Fc fragment of an antibody.

In some embodiments, a target may be a marker that is exclusively or primarily associated with a target cell, or one or more tissue types, with one or more cell types, with one or more diseases, and/or with one or more developmental stages. In some embodiments, a target can comprise a protein (e.g., a cell surface receptor, transmembrane protein, glycoprotein, etc.), a carbohydrate (e.g., a glycan moiety, glycocalyx, etc.), a lipid (e.g., steroid, phospholipid, etc.), and/or a nucleic acid (e.g., a DNA, RNA, etc.).

In yet other embodiments, X is a moiety described in the Therapeutic Target Database, see, e.g., Zhu et al., Update of TTD: Therapeutic Target Database, Nucleic Acids Res. 38 (1): 787-91 (2010), or a moiety that targets one or more of the proteins, nucleic acids, diseases or pathways described therein.

In some embodiments, the target, target cell or marker is a molecule that is present exclusively or predominantly on malignant cells, e.g., a tumor antigen. In some embodiments, a marker is a prostate cancer marker. In certain embodiments, the prostate cancer marker is prostate specific membrane antigen (PSMA), a 100 kDa transmembrane glycoprotein that is expressed in most prostatic tissues, but is more highly expressed in prostatic cancer tissue than in normal tissue. PSMA is a well established tumor marker that is up-regulated in prostate cancer, particularly in advanced, hormone-independent, and metastatic disease (Ghosh and Heston, 2004, J. Cell. Biochem., 91 :528-539). PSMA has been employed as a tumor marker for imaging of metastatic prostate cancer and as a target for experimental immunotherapeutic agents. PSMA is the molecular target of PROSTASCINT®, a monoclonal antibody-based imaging agent approved for diagnostic imaging of prostate cancer metastases. PSMA is differentially expressed at high levels on the neovasculature of most non-prostate solid tumors, including breast and lung cancers. PSMA targeting for non-prostate cancers has been demonstrated in clinical trials (Morris et al., 2007, Clin. Cancer Res., 13:2707-13; Milowsky et al, 2007, J. Clin. Oncol, 25:540-547). Therefore, the highly restricted presence of PSMA on prostate cancer cells and non-prostate solid tumor neovasculature makes it an attractive target for delivery of cytotoxic agents to most solid tumors.

In other embodiments, a marker is a breast cancer marker, a colon cancer marker, a rectal cancer marker, a lung cancer marker, a pancreatic cancer marker, a ovarian cancer marker, a bone cancer marker, a renal cancer marker, a liver cancer marker, a neurological cancer marker, a gastric cancer marker, a testicular cancer marker, a head and neck cancer marker, an esophageal cancer marker, or a cervical cancer marker.

Other cell surface markers are useful as potential targets for tumor-homing therapeutics, including, for example HER-2, HER-3, EGFR, and the folate receptor.

In other embodiments, the targeting moiety binds a target such as CD19, CD70, CD56, PSMA, alpha integrin, CD22, CD138, EphA2, AGS-5, Nectin-4, HER2, GPMNB, CD74 and Le.

In certain embodiments, the targeting moiety or moieties of the conjugate are present at a predetermined molar weight percentage from about 1% to 10%, or about 10% to about 20%, or about 20% to about 30%, or about 30% to 40%, or about 40% to 50%, or about 50% to 60%, or about 60% to 70%, or about 70% to 80%, or about 80% to 90%, or about 90% to 99% such that the sum of the molar weight percentages of the components of the conjugate is 100%. The amount of targeting moieties of the conjugate may also be expressed in terms of proportion to the active agent(s), for example, in a ratio of ligand to active agent of about 10:1, 9:1, 8:1, 7:1, 6:1, 5:1, 4:1, 3:1, 2:1, 1:1, 1:2, 1:3, 1:4; 1:5, 1:6, 1:7, 1:8, 1:9, or 1:10.

### C. Linkers

The conjugates contain one or more linkers attaching the active agents and targeting moieties. The linker, Y, can be bound to an active agent and a targeting ligand to form a conjugate wherein the conjugate releases at least one active agent upon delivery to a target cell. The linker can be a C₁-C₁₀ straight chain alkyl, C₁-C₁₀ straight chain O-alkyl, C₁-C₁₀ straight chain substituted alkyl, C₁-C₁₀ straight chain substituted O-alkyl, C₄-C₁₃ branched chain alkyl, C₄-C₁₃ branched chain O-alkyl, C₂-C₁₂ straight chain alkenyl, C₂-C₁₂ straight chain O-alkenyl, C₃-C₁₂ straight chain substituted alkenyl, C₃-C₁₂ straight chain substituted O-alkenyl, polyethylene glycol, polylactic acid, polyglycolic acid, poly(lactide-co-glycolide), polycarprolactone, polycyanoacrylate, ketone, aryl, heterocyclic, succinic ester, amino acid, aromatic group, ether, crown ether, urea, thiourea, amide, purine, pyrimidine, bypiridine, indole derivative acting as a cross linker, chelator, aldehyde, ketone, bisamine, bis alcohol, heterocyclic ring structure, azirine, disulfide, thioether, hydrazone and combinations thereof. For example, the linker can be a C₃ straight chain alkyl or a ketone. The alkyl chain of the linker can be substituted with one or more substituents or heteroatoms. In some embodiments the linker contains one or more atoms or groups selected from —O—, —C(═O)—, —NR, —O—C(═O)—NR—, —S—, —S—S—. The linker may be selected from dicarboxylate derivatives of succinic acid, glutaric acid or diglycolic acid.

In some embodiments the alkyl chain of the linker may optionally be interrupted by one or more atoms or groups selected from —O—, —C(═O)—, —NR, —O—C(═O)—NR—, —S—, —S—S—. The linker may be selected from dicarboxylate derivatives of succinic acid, glutaric acid or diglycolic acid.

### III. Particles

Particles containing one or more conjugates can be polymeric particles, lipid particles, solid lipid particles, inorganic particles, or combinations thereof (e.g., lipid stabilized polymeric particles). In preferred embodiments, the particles are polymeric particles or contain a polymeric matrix. The particles can contain any of the polymers described herein or derivatives or copolymers thereof. The particles will generally contain one or more biocompatible polymers. The polymers can be biodegradable polymers. The polymers can be hydrophobic polymers, hydrophilic polymers, or amphiphilic polymers. In some embodiments, the particles contain one or more polymers having an additional targeting moiety attached thereto.

The size of the particles can be adjusted for the intended application. The particles can be nanoparticles or microparticles, although nanoparticles are preferred. The particle can have a diameter of about 10 nm to about 10 microns, about 10 nm to about 1 micron, about 10 nm to about 500 nm, about 20 nm to about 500 nm, or about 25 nm to about 250 nm. In preferred embodiments the particle is a nanoparticle having a diameter from about 25 nm to about 250 nm.

In various embodiments, a particle may be a nanoparticle, i.e., the particle has a characteristic dimension of less than about 1 micrometer, where the characteristic dimension of a particle is the diameter of a perfect sphere having the same volume as the particle. The plurality of particles can be characterized by an average diameter (e.g., the average diameter for the plurality of particles). In some embodiments, the diameter of the particles may have a Gaussian-type distribution. In some embodiments, the plurality of particles have an average diameter of less than about 300 nm, less than about 250 nm, less than about 200 nm, less than about 150 nm, less than about 100 nm, less than about 50 nm, less than about 30 nm, less than about 10 nm, less than about 3 nm, or less than about 1 nm. In some embodiments, the particles have an average diameter of at least about 5 nm, at least about 10 nm, at least about 30 nm, at least about 50 nm, at least about 100 nm, at least about 150 nm, or greater. In certain embodiments, the plurality of the particles have an average diameter of about 10 nm, about 25 nm, about 50 nm, about 100 nm, about 150 nm, about 200 nm, about 250 nm, about 300 nm, about 500 nm, or the like. In some embodiments, the plurality of particles have an average diameter between about 10 nm and about 500 nm, between about 50 nm and about 400 nm, between about 100 nm and about 300 nm, between about 150 nm and about 250 nm, between about 175 nm and about 225 nm, or the like. In some embodiments, the plurality of particles have an average diameter between about 10 nm and about 500 nm, between about 20 nm and about 400 nm, between about 30 nm and about 300 nm, between about 40 nm and about 200 nm, between about 50 nm and about 175 nm, between about 60 nm and about 150 nm, between about 70 nm and about 130 nm, or the like. For example, the average diameter can be between about 70 nm and 130 nm. In some embodiments, the plurality of particles have an average diameter between about 20 nm and about 220 nm, between about 30 nm and about 200 nm, between about 40 nm and about 180 nm, between about 50 nm and about 170 nm, between about 60 nm and about 150 nm, or between about 70 nm and about 130 nm. In one embodiment, the particles have a size of 40 to 120 nm with a zeta potential close to 0 mV at low to zero ionic strengths (1 to 10 mM), with zeta potential values between +5 to −5 mV, and a zero/neutral or a small −ve surface charge.

### A. Conjugates

The particles contain one or more conjugates as described above. The conjugates can be present on the interior of the particle, on the exterior of the particle, or both.

### B. Polymers

The particles can contain one more of the following polyesters: homopolymers including glycolic acid units, referred to herein as “PGA”, and lactic acid units, such as poly-L-lactic acid, poly-D-lactic acid, poly-D,L-lactic acid, poly-L-lactide, poly-D-lactide, and poly-D,L-lactide, collectively referred to herein as “PLA”, and caprolactone units, such as poly(ε-caprolactone), collectively referred to herein as “PCL”; and copolymers including lactic acid and glycolic acid units, such as various forms of poly(lactic acid-co-glycolic acid) and poly(lactide-co-glycolide) characterized by the ratio of lactic acid:glycolic acid, collectively referred to herein as “PLGA”; and polyacrylates, and derivatives thereof. Exemplary polymers also include copolymers of polyethylene glycol (PEG) and the aforementioned polyesters, such as various forms of PLGA-PEG or PLA-PEG copolymers, collectively referred to herein as “PEGylated polymers”. In certain embodiments, the PEG region can be covalently associated with polymer to yield “PEGylated polymers” by a cleavable linker.

The particles can contain one or more hydrophilic polymers. Hydrophilic polymers include cellulosic polymers such as starch and polysaccharides; hydrophilic polypeptides; poly(amino acids) such as poly-L-glutamic acid (PGS), gamma-polyglutamic acid, poly-L-aspartic acid, poly-L-serine, or poly-L-lysine; polyalkylene glycols and polyalkylene oxides such as polyethylene glycol (PEG), polypropylene glycol (PPG), and poly(ethylene oxide) (PEO); poly(oxyethylated polyol); poly(olefinic alcohol); polyvinylpyrrolidone); poly(hydroxyalkylmethacrylamide); poly(hydroxyalkylmethacrylate); poly(saccharides); poly(hydroxy acids); poly(vinyl alcohol), and copolymers thereof.

The particles can contain one or more hydrophobic polymers. Examples of suitable hydrophobic polymers include polyhydroxyacids such as poly(lactic acid), poly(glycolic acid), and poly(lactic acid-co-glycolic acids); polyhydroxyalkanoates such as poly3-hydroxybutyrate or poly4-hydroxybutyrate; polycaprolactones; poly(orthoesters); polyanhydrides; poly(phosphazenes); poly(lactide-co-caprolactones); polycarbonates such as tyrosine polycarbonates; polyamides (including synthetic and natural polyamides), polypeptides, and poly(amino acids); polyesteramides; polyesters; poly(dioxanones); poly(alkylene alkylates); hydrophobic polyethers; polyurethanes; polyetheresters; polyacetals; polycyanoacrylates; polyacrylates; polymethylmethacrylates; polysiloxanes; poly(oxyethylene)/poly(oxypropylene) copolymers; polyketals; polyphosphates; polyhydroxyvalerates; polyalkylene oxalates; polyalkylene succinates; poly(maleic acids), as well as copolymers thereof.

In certain embodiments, the hydrophobic polymer is an aliphatic polyester. In some embodiments, the hydrophobic polymer is poly(lactic acid), poly(glycolic acid), or poly(lactic acid-co-glycolic acid).

The particles can contain one or more biodegradable polymers. Biodegradable polymers can include polymers that are insoluble or sparingly soluble in water that are converted chemically or enzymatically in the body into water-soluble materials. Biodegradable polymers can include soluble polymers crosslinked by hydolyzable cross-linking groups to render the crosslinked polymer insoluble or sparingly soluble in water.

Biodegradable polymers in the particle can include polyamides, polycarbonates, polyalkylenes, polyalkylene glycols, polyalkylene oxides, polyalkylene terepthalates, polyvinyl alcohols, polyvinyl ethers, polyvinyl esters, polyvinyl halides, polyvinylpyrrolidone, polyglycolides, polysiloxanes, polyurethanes and copolymers thereof, alkyl cellulose such as methyl cellulose and ethyl cellulose, hydroxyalkyl celluloses such as hydroxypropyl cellulose, hydroxy-propyl methyl cellulose, and hydroxybutyl methyl cellulose, cellulose ethers, cellulose esters, nitro celluloses, cellulose acetate, cellulose propionate, cellulose acetate butyrate, cellulose acetate phthalate, carboxylethyl cellulose, cellulose triacetate, cellulose sulphate sodium salt, polymers of acrylic and methacrylic esters such as poly (methyl methacrylate), poly(ethylmethacrylate), poly(butylmethacrylate), poly(isobutylmethacrylate), poly(hexlmethacrylate), poly(isodecylmethacrylate), poly(lauryl methacrylate), poly (phenyl methacrylate), poly(methyl acrylate), poly(isopropyl acrylate), poly(isobutyl acrylate), poly(octadecyl acrylate), polyethylene, polypropylene poly(ethylene glycol), poly(ethylene oxide), poly(ethylene terephthalate), poly(vinyl alcohols), poly(vinyl acetate, poly vinyl chloride polystyrene and polyvinylpryrrolidone, derivatives thereof, linear and branched copolymers and block copolymers thereof, and blends thereof. Exemplary biodegradable polymers include polyesters, poly(ortho esters), poly(ethylene imines), poly(caprolactones), poly(hydroxyalkanoates), poly(hydroxyvalerates), polyanhydrides, poly(acrylic acids), polyglycolides, poly(urethanes), polycarbonates, polyphosphate esters, polyphosphazenes, derivatives thereof, linear and branched copolymers and block copolymers thereof, and blends thereof. In particularly preferred embodiments the nanoparticle contains biodegradable polyesters or polyanhydrides such as poly(lactic acid), poly(glycolic acid), and poly(lactic-co-glycolic acid).

The particles can contain one or more amphiphilic polymers. Amphiphilic polymers can be polymers containing a hydrophobic polymer block and a hydrophilic polymer block. The hydrophobic polymer block can contain one or more of the hydrophobic polymers above or a derivative or copolymer thereof. The hydrophilic polymer block can contain one or more of the hydrophilic polymers above or a derivative or copolymer thereof. In some embodiments the amphiphilic polymer is a di-block polymer containing a hydrophobic end formed from a hydrophobic polymer and a hydrophilic end formed of a hydrophilic polymer. In some embodiments, a moiety can be attached to the hydrophobic end, to the hydrophilic end, or both. The nanoparticle can contain two or more amphiphilic polymers.

### C. Lipids

The particles can contain one or more lipids or amphiphilic compounds. For example, the particles can be liposomes, lipid micelles, solid lipid particles, or lipid-stabilized polymeric particles. The lipid particle can be made from one or a mixture of different lipids. Lipid particles are formed from one or more lipids, which can be neutral, anionic, or cationic at physiologic pH. The lipid particle is preferably made from one or more biocompatible lipids. The lipid particles may be formed from a combination of more than one lipid, for example, a charged lipid may be combined with a lipid that is non-ionic or uncharged at physiological pH.

The particle can be a lipid micelle. Lipid micelles for drug delivery are known in the art. Lipid micelles can be formed, for instance, as a water-in-oil emulsion with a lipid surfactant. An emulsion is a blend of two immiscible phases wherein a surfactant is added to stabilize the dispersed droplets. In some embodiments the lipid micelle is a microemulsion. A microemulsion is a thermodynamically stable system composed of at least water, oil and a lipid surfactant producing a transparent and thermodynamically stable system whose droplet size is less than 1 micron, from about 10 nm to about 500 nm, or from about 10 nm to about 250 nm. Lipid micelles are generally useful for encapsulating hydrophobic active agents, including hydrophobic therapeutic agents, hydrophobic prophylactic agents, or hydrophobic diagnostic agents.

The particle can be a liposome. Liposomes are small vesicles composed of an aqueous medium surrounded by lipids arranged in spherical bilayers. Liposomes can be classified as small unilamellar vesicles, large unilamellar vesicles, or multi-lamellar vesicles. Multi-lamellar liposomes contain multiple concentric lipid bilayers. Liposomes can be used to encapsulate agents, by trapping hydrophilic agents in the aqueous interior or between bilayers, or by trapping hydrophobic agents within the bilayer.

The lipid micelles and liposomes typically have an aqueous center. The aqueous center can contain water or a mixture of water and alcohol. Suitable alcohols include, but are not limited to, methanol, ethanol, propanol, (such as isopropanol), butanol (such as n-butanol, isobutanol, sec-butanol, tent-butanol, pentanol (such as amyl alcohol, isobutyl carbinol), hexanol (such as 1-hexanol, 2-hexanol, 3-hexanol), heptanol (such as 1-heptanol, 2-heptanol, 3-heptanol and 4-heptanol) or octanol (such as 1-octanol) or a combination thereof.

The particle can be a solid lipid particle. Solid lipid particles present an alternative to the colloidal micelles and liposomes. Solid lipid particles are typically submicron in size, i.e. from about 10 nm to about 1 micron, from 10 nm to about 500 nm, or from 10 nm to about 250 nm. Solid lipid particles are formed of lipids that are solids at room temperature. They are derived from oil-in-water emulsions, by replacing the liquid oil by a solid lipid.

Suitable neutral and anionic lipids include, but are not limited to, sterols and lipids such as cholesterol, phospholipids, lysolipids, lysophospholipids, sphingolipids or pegylated lipids. Neutral and anionic lipids include, but are not limited to, phosphatidylcholine (PC) (such as egg PC, soy PC), including 1 ,2-diacyl-glycero-3-phosphocholines; phosphatidylserine (PS), phosphatidylglycerol, phosphatidylinositol (PI); glycolipids; sphingophospholipids such as sphingomyelin and sphingoglycolipids (also known as 1-ceramidyl glucosides) such as ceramide galactopyranoside, gangliosides and cerebrosides; fatty acids, sterols, containing a carboxylic acid group for example, cholesterol; 1 ,2-diacyl-sn-glycero-3-phosphoethanolamine, including, but not limited to, 1 ,2-dioleylphosphoethanolamine (DOPE), 1 ,2-dihexadecylphosphoethanolamine (DHPE), 1 ,2-di stearoylphosphatidylcholine (DSPC), 1 ,2-dipalmitoyl phosphatidylcholine (DPPC), and 1 ,2-dimyristoylphosphatidylcholine (DMPC). The lipids can also include various natural (e.g., tissue derived L-α-phosphatidyl: egg yolk, heart, brain, liver, soybean) and/or synthetic (e.g., saturated and unsaturated 1,2-diacyl-sn-glycero-3-phosphocholines, 1-acyl-2-acyl-sn-glycero-3-phosphocholines, 1,2-diheptanoyl-SN-glycero-3-phosphocholine) derivatives of the lipids.

Suitable cationic lipids include, but are not limited to, N-[1-(2,3-dioleoyloxy)propyl]-N,N,N-trimethyl ammonium salts, also references as TAP lipids, for example methylsulfate salt. Suitable TAP lipids include, but are not limited to, DOTAP (dioleoyl-), DMTAP (dimyristoyl-), DPTAP (dipalmitoyl-), and DSTAP (distearoyl-). Suitable cationic lipids in the liposomes include, but are not limited to, dimethyldioctadecyl ammonium bromide (DDAB), 1 ,2-diacyloxy-3-trimethylammonium propanes, N-[1-(2,3-dioloyloxy)propyl]-N,N-dimethyl amine (DODAP), 1 ,2-diacyloxy-3-dimethylammonium propanes, N-[1-(2,3-dioleyloxy)propyl]-N,N,N-trimethylammonium chloride (DOTMA), 1 ,2-dialkyloxy-3-dimethylammonium propanes, dioctadecylamidoglycylspermine (DOGS), 3-[N-(N′,N′-dimethylamino-ethane)carbamoyl]cholesterol (DC-Chol); 2,3-dioleoyloxy-N-(2-(sperminecarboxamido)-ethyl)-N,N-dimethyl-1-propanaminium trifluoro-acetate (DOSPA), β-alanyl cholesterol, cetyl trimethyl ammonium bromide (CTAB), diC₁₄-amidine, N-ferf-butyl-N′-tetradecyl-3-tetradecylamino-propionamidine, N-(alpha-trimethylammonioacetyl)didodecyl-D-glutamate chloride (TMAG), ditetradecanoyl-N-(trimethylammonio-acetyl)diethanolamine chloride, 1 ,3-dioleoyloxy-2-(6-carboxy-spermyl)-propylamide (DOSPER), and N , N , N′, N′-tetramethyl-, N′-bis(2-hydroxylethyl)-2,3-dioleoyloxy-1 ,4-butanediammonium iodide. In one embodiment, the cationic lipids can be 1-[2-(acyloxy)ethyl]2-alkyl(alkenyl)-3-(2-hydroxyethyl)-imidazolinium chloride derivatives, for example, 1-[2-(9(Z)-octadecenoyloxy)ethyl]-2-(8(Z)-heptadecenyl-3-(2-hydroxyethyl)imidazolinium chloride (DOTIM), and 1-[2-(hexadecanoyloxy)ethyl]-2-pentadecyl-3-(2-hydroxyethyl)imidazolinium chloride (DPTIM). In one embodiment, the cationic lipids can be 2,3-dialkyloxypropyl quaternary ammonium compound derivatives containing a hydroxyalkyl moiety on the quaternary amine, for example, 1 ,2-dioleoyl-3-dimethyl-hydroxyethyl ammonium bromide (DORI), 1 ,2-dioleyloxypropyl-3-dimethyl-hydroxyethyl ammonium bromide (DORIE), 1 ,2-dioleyloxypropyl-3-dimetyl-hydroxypropyl ammonium bromide (DOME-HP), 1 ,2-dioleyl-oxy-propyl-3-dimethyl-hydroxybutyl ammonium bromide (DORIE-HB), 1 ,2-dioleyloxypropyl-3-dimethyl-hydroxypentyl ammonium bromide (DORIE-Hpe), 1 ,2-dimyristyloxypropyl-3-dimethyl-hydroxylethyl ammonium bromide (DMRIE), 1 ,2-dipalmityloxypropyl-3-dimethyl-hydroxyethyl ammonium bromide (DPRIE), and 1 ,2-disteryloxypropyl-3-dimethyl-hydroxyethyl ammonium bromide (DSRIE).

Suitable solid lipids include, but are not limited to, higher saturated alcohols, higher fatty acids, sphingolipids, synthetic esters, and mono-, di-, and triglycerides of higher saturated fatty acids. Solid lipids can include aliphatic alcohols having 10-40, preferably 12-30 carbon atoms, such as cetostearyl alcohol. Solid lipids can include higher fatty acids of 10-40, preferably 12-30 carbon atoms, such as stearic acid, palmitic acid, decanoic acid, and behenic acid. Solid lipids can include glycerides, including monoglycerides, diglycerides, and triglycerides, of higher saturated fatty acids having 10-40, preferably 12-30 carbon atoms, such as glyceryl monostearate, glycerol behenate, glycerol palmitostearate, glycerol trilaurate, tricaprin, trilaurin, trimyristin, tripalmitin, tristearin, and hydrogenated castor oil. Suitable solid lipids can include cetyl palmitate, beeswax, or cyclodextrin.

Amphiphilic compounds include, but are not limited to, phospholipids, such as 1,2 distearoyl-sn-glycero-3-phosphoethanolamine (DSPE), dipalmitoylphosphatidylcholine (DPPC), di stearoylphosphatidylcholine (DSPC), diarachidoylphosphatidylcholine (DAPC), dibehenoylphosphatidylcholine (DBPC), ditricosanoylphosphatidylcholine (DTPC), and dilignoceroylphatidylcholine (DLPC), incorporated at a ratio of between 0.01-60 (weight lipid/w polymer), most preferably between 0.1-30 (weight lipid/w polymer). Phospholipids which may be used include, but are not limited to, phosphatidic acids, phosphatidyl cholines with both saturated and unsaturated lipids, phosphatidyl ethanolamines, phosphatidylglycerols, phosphatidylserines, phosphatidylinositols, lysophosphatidyl derivatives, cardiolipin, and β-acyl-y-alkyl phospholipids. Examples of phospholipids include, but are not limited to, phosphatidylcholines such as dioleoylphosphatidylcholine, dimyristoylphosphatidylcholine, dipentadecanoylphosphatidylcholine dilauroylphosphatidylcholine, dipalmitoylphosphatidylcholine (DPPC), di stearoylphosphatidylcholine (DSPC), diarachidoylphosphatidylcholine (DAPC), dibehenoylphosphatidylcho-line (DBPC), ditricosanoylphosphatidylcholine (DTPC), dilignoceroylphatidylcholine (DLPC); and phosphatidylethanolamines such as dioleoylphosphatidylethanolamine or 1-hexadecyl-2-palmitoylglycerophos-phoethanolamine. Synthetic phospholipids with asymmetric acyl chains (e.g., with one acyl chain of 6 carbons and another acyl chain of 12 carbons) may also be used.

### D. Additional Active Agents

The particles can contain one or more additional active agents in addition to those in the conjugates. The additional active agents can be therapeutic, prophylactic, diagnostic, or nutritional agents as listed above. The additional active agents can be present in any amount, e.g. from 1% to 90%, from 1% to 50%, from 1% to 25%, from 1% to 20%, from 1% to 10%, or from 5% to 10% (w/w) based upon the weight of the particle. In one embodiment, the agents are incorporated in a 1% to 10% loading w/w.

### E. Additional Targeting Moieties

The particles can contain one or more targeting moieties targeting the particle to a specific organ, tissue, cell type, or subcellular compartment in addition to the targeting moieties of the conjugate. The additional targeting moieties can be present on the surface of the particle, on the interior of the particle, or both. The additional targeting moieties can be immobilized on the surface of the particle, e.g., can be covalently attached to polymer or lipid in the particle. In preferred embodiments, the additional targeting moieties are covalently attached to an amphiphilic polymer or a lipid such that the targeting moieties are oriented on the surface of the particle.

### IV. Formulations

The formulations described herein contain an effective amount of nanoparticles in a pharmaceutical carrier appropriate for administration to an individual in need thereof. The formulations are generally administered parenterally (e.g., by injection or infusion). The formulations or variations thereof may be administered in any manner including enterally, topically (e.g., to the eye), or via pulmonary administration. In some embodiments the formulations are administered topically.

### A. Parenteral Formulations

The nanoparticles can be formulated for parenteral delivery, such as injection or infusion, in the form of a solution, suspension or emulsion. The formulation can be administered systemically, regionally or directly to the organ or tissue to be treated.

Parenteral formulations can be prepared as aqueous compositions using techniques is known in the art. Typically, such compositions can be prepared as injectable formulations, for example, solutions or suspensions; solid forms suitable for using to prepare solutions or suspensions upon the addition of a reconstitution medium prior to injection; emulsions, such as water-in-oil (w/o) emulsions, oil-in-water (o/w) emulsions, and microemulsions thereof, liposomes, or emulsomes.

The carrier can be a solvent or dispersion medium containing, for example, water, ethanol, one or more polyols (e.g., glycerol, propylene glycol, and liquid polyethylene glycol), oils, such as vegetable oils (e.g., peanut oil, corn oil, sesame oil, etc.), and combinations thereof. The proper fluidity can be maintained, for example, by the use of a coating, such as lecithin, by the maintenance of the required particle size in the case of dispersion and/or by the use of surfactants. In many cases, it will be preferable to include isotonic agents, for example, sugars or sodium chloride.

Solutions and dispersions of the nanoparticles can be prepared in water or another solvent or dispersing medium suitably mixed with one or more pharmaceutically acceptable excipients including, but not limited to, surfactants, dispersants, emulsifiers, pH modifying agents, and combinations thereof.

Suitable surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-β-alanine, sodium N-lauryl-β-iminodipropionate, myristoamphoacetate, lauryl betaine and lauryl sulfobetaine.

The formulation can contain a preservative to prevent the growth of microorganisms. Suitable preservatives include, but are not limited to, parabens, chlorobutanol, phenol, sorbic acid, and thimerosal. The formulation may also contain an antioxidant to prevent degradation of the active agent(s) or nanoparticles.

The formulation is typically buffered to a pH of 3-8 for parenteral administration upon reconstitution. Suitable buffers include, but are not limited to, phosphate buffers, acetate buffers, and citrate buffers. If using 10% sucrose or 5% dextrose, a buffer may not be required.

Water soluble polymers are often used in formulations for parenteral administration. Suitable water-soluble polymers include, but are not limited to, polyvinylpyrrolidone, dextran, carboxymethylcellulose, and polyethylene glycol.

Sterile injectable solutions can be prepared by incorporating the nanoparticles in the required amount in the appropriate solvent or dispersion medium with one or more of the excipients listed above, as required, followed by filtered sterilization. Generally, dispersions are prepared by incorporating the various sterilized nanoparticles into a sterile vehicle which contains the basic dispersion medium and the required other ingredients from those listed above. In the case of sterile powders for the preparation of sterile injectable solutions, the preferred methods of preparation are vacuum-drying and freeze-drying techniques which yield a powder of the nanoparticle plus any additional desired ingredient from a previously sterile-filtered solution thereof. The powders can be prepared in such a manner that the particles are porous in nature, which can increase dissolution of the particles. Methods for making porous particles are well known in the art.

Pharmaceutical formulations for parenteral administration can be in the form of a sterile aqueous solution or suspension of particles formed from one or more polymer-drug conjugates. Acceptable solvents include, for example, water, Ringer's solution, phosphate buffered saline (PBS), and isotonic sodium chloride solution. The formulation may also be a sterile solution, suspension, or emulsion in a nontoxic, parenterally acceptable diluent or solvent such as 1,3-butanediol.

In some instances, the formulation is distributed or packaged in a liquid form. Alternatively, formulations for parenteral administration can be packed as a solid, obtained, for example by lyophilization of a suitable liquid formulation. The solid can be reconstituted with an appropriate carrier or diluent prior to administration.

Solutions, suspensions, or emulsions for parenteral administration may be buffered with an effective amount of buffer necessary to maintain a pH suitable for ocular administration. Suitable buffers are well known by those skilled in the art and some examples of useful buffers are acetate, borate, carbonate, citrate, and phosphate buffers.

Solutions, suspensions, or emulsions for parenteral administration may also contain one or more tonicity agents to adjust the isotonic range of the formulation. Suitable tonicity agents are well known in the art and some examples include glycerin, sucrose, dextrose, mannitol, sorbitol, sodium chloride, and other electrolytes.

Solutions, suspensions, or emulsions for parenteral administration may also contain one or more preservatives to prevent bacterial contamination of the ophthalmic preparations. Suitable preservatives are known in the art, and include polyhexamethylenebiguanidine (PHMB), benzalkonium chloride (BAK), stabilized oxychloro complexes (otherwise known as Purite®), phenylmercuric acetate, chlorobutanol, sorbic acid, chlorhexidine, benzyl alcohol, parabens, thimerosal, and mixtures thereof.

Solutions, suspensions, or emulsions for parenteral administration may also contain one or more excipients known art, such as dispersing agents, wetting agents, and suspending agents.

### B. Mucosal Topical Formulations

The nanoparticles can be formulated for topical administration to a mucosal surface Suitable dosage forms for topical administration include creams, ointments, salves, sprays, gels, lotions, emulsions, liquids, and transdermal patches. The formulation may be formulated for transmucosal transepithelial, or transendothelial administration. The compositions contain one or more chemical penetration enhancers, membrane permeability agents, membrane transport agents, emollients, surfactants, stabilizers, and combination thereof. In some embodiments, the nanoparticles can be administered as a liquid formulation, such as a solution or suspension, a semi-solid formulation, such as a lotion or ointment, or a solid formulation. In some embodiments, the nanoparticles are formulated as liquids, including solutions and suspensions, such as eye drops or as a semi-solid formulation, to the mucosa, such as the eye or vaginally or rectally.

“Surfactants” are surface-active agents that lower surface tension and thereby increase the emulsifying, foaming, dispersing, spreading and wetting properties of a product. Suitable non-ionic surfactants include emulsifying wax, glyceryl monooleate, polyoxyethylene alkyl ethers, polyoxyethylene castor oil derivatives, polysorbate, sorbitan esters, benzyl alcohol, benzyl benzoate, cyclodextrins, glycerin monostearate, poloxamer, povidone and combinations thereof. In one embodiment, the non-ionic surfactant is stearyl alcohol.

“Emulsifiers” are surface active substances which promote the suspension of one liquid in another and promote the formation of a stable mixture, or emulsion, of oil and water. Common emulsifiers are: metallic soaps, certain animal and vegetable oils, and various polar compounds. Suitable emulsifiers include acacia, anionic emulsifying wax, calcium stearate, carbomers, cetostearyl alcohol, cetyl alcohol, cholesterol, diethanolamine, ethylene glycol palmitostearate, glycerin monostearate, glyceryl monooleate, hydroxpropyl cellulose, hypromellose, lanolin, hydrous, lanolin alcohols, lecithin, medium-chain triglycerides, methylcellulose, mineral oil and lanolin alcohols, monobasic sodium phosphate, monoethanolamine, nonionic emulsifying wax, oleic acid, poloxamer, poloxamers, polyoxyethylene alkyl ethers, polyoxyethylene castor oil derivatives, polyoxyethylene sorbitan fatty acid esters, polyoxyethylene stearates, propylene glycol alginate, self-emulsifying glyceryl monostearate, sodium citrate dehydrate, sodium lauryl sulfate, sorbitan esters, stearic acid, sunflower oil, tragacanth, triethanolamine, xanthan gum and combinations thereof. In one embodiment, the emulsifier is glycerol stearate.

Suitable classes of penetration enhancers are known in the art and include, but are not limited to, fatty alcohols, fatty acid esters, fatty acids, fatty alcohol ethers, amino acids, phospholipids, lecithins, cholate salts, enzymes, amines and amides, complexing agents (liposomes, cyclodextrins, modified celluloses, and diimides), macrocyclics, such as macrocylic lactones, ketones, and anhydrides and cyclic ureas, surfactants, N-methyl pyrrolidones and derivatives thereof, DMSO and related compounds, ionic compounds, azone and related compounds, and solvents, such as alcohols, ketones, amides, polyols (e.g., glycols). Examples of these classes are known in the art.

### V. Methods of Making Conjugates

The conjugates can be made by many different synthetic procedures. The conjugates can be prepared from linkers having one or more reactive coupling groups or from one or more linker precursors capable of reacting with a reactive coupling group on an active agent or targeting moiety to form a covalent bond.

The conjugates can be prepared from a linker precursor capable of reacting with a reactive coupling group on an active agent or targeting moiety to form the linker covalently bonded to the active agent or targeting moiety.

The linker precursor can be a diacid or substituted diacid. Diacids, as used herein, can refer to substituted or unsubstituted alkyl, heteroalkyl, aryl, or heteroaryl compounds having two or more carboxylic acid groups, preferably having between 2 and 50, between 2 and 30, between 2 and 12, or between 2 and 8 carbon atoms. Suitable diacids can include oxalic acid, malonic acid, succinic acid, glutaric acid, adipic acid, pimelic acid, suberic acid, azelaic acid, sebacic acid, phthalic acid, iso-phthalic acid, terepthalic acid, and derivatives thereof.

The linker precursor can be an activated diacid derivative such as a diacid anhydride, diacid ester, or diacid halide. The diacid anhydride can be a cyclic anhydride obtained from the intramolecular dehydration of a diacid or diacid derivative such as those described above. The diacid anhydride can be malonic anhydride, succinic anhydride, glutaric anhydride, adipic anhydride, pimelic anhydride, phthalic anhydride, diglycolic anhydride, or a derivative thereof; preferably succinic anhydride, diglycolic anhydride, or a derivative thereof. The diacid ester can be an activated ester of any of the diacids described above, including methyl and butyl diesters or bis-(p-nitrophenyl) diesters of oxalic acid, malonic acid, succinic acid, glutaric acid, adipic acid, pimelic acid, suberic acid, azelaic acid, sebacic acid, phthalic acid, iso-phthalic acid, terepthalic acid, and derivatives thereof. The diacid halide can include the corresponding acid fluorides, acid chlorides, acid bromides, or acid iodides of the diacids described above. In preferred embodiments the diacid halide is succinyl chloride or diglycolyl chloride. For example, a therapeutic agent having a reactive (—OH) coupling group and a targeting moiety having a reactive (—NH2) coupling group can be used to prepare a conjugate having a disuccinate linker according to the following general scheme.

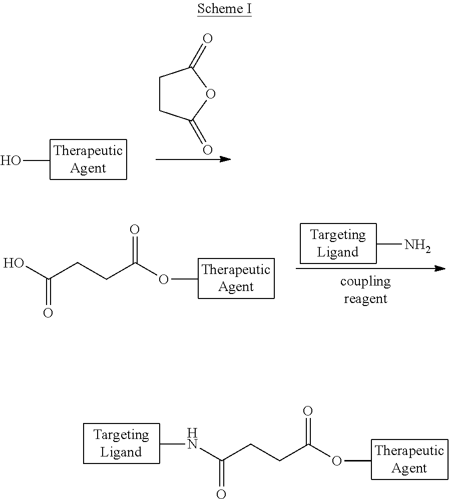

Referring to Scheme I above, the conjugates can be prepared by providing an active agent having a hydroxyl group and reacting it with a succinic anhydride linker precursor to form the conjugate of active agent—succinate-SSPy. A targeting moiety with an available —NH₂ group is reacted with a coupling reagent and the active agent—succinate-SSPy to form the targeting moiety—linker—active agent conjugate.

Other functional groups that can be linked to include, but are not limited to, —SH, —COOH, alkenyl, phosphate, sulfate, heterocyclic NH, alkyne and ketone.

The coupling reaction can be carried out under esterification conditions known to those of ordinary skill in the art such as in the presence of activating agents, e.g., carbodiimides (such as diisopropoylcarbodiimide (DIPC)), with or without catalyst such as dimethylaminopyridine (DMAP). This reaction can be carried out in an appropriate solvent, such as dichloromethane, chloroform or ethyl acetate, at a temperature or between about 0° C. and the reflux temperature of the solvent (e.g., ambient temperature). The coupling reaction is generally performed in a solvent such as pyridine or in a chlorinated solvent in the presence of a catalyst such as DMAP or pyridine at a temperature between about 0° C. and the reflux temperature of the solvent (e.g., ambient temperature). In preferred embodiments, the coupling reagent is selected from the group consisting of 4-(2-pyridyldithio)-butanoic acid, and a carbodiimide coupling reagent such as DCC in a chlorinated, ethereal or amidic solvent (such as N,N-dimethylformamide) in the presence of a catalyst such as DMAP at a temperature between about 0° C. and the reflux temperature of the solvent (e.g., ambient temperature).

The conjugates can be prepared by coupling an active agent and/or targeting moiety having one or more reactive coupling groups to a linker having complimentary reactive groups capable of reacting with the reactive coupling groups on the active agent or targeting moiety to form a covalent bond. For example, an active agent or targeting moiety having a primary amine group can be coupled to a linker having an isothiocyonate group or another amine-reactive coupling group. In some embodiments the linker contains a first reactive coupling group capable of reacting with a complimentary functional group on the active agent and a second reactive coupling group different from the first and capable of reacting with a complimentary group on the targeting moiety. In some embodiments one or both of the reactive coupling groups on the linker can be protected with a suitable protecting group during part of the synthesis.

### VI. Methods of Making Particles

In various embodiments, a method of making the particles includes providing a conjugate; providing a base component such as PLA-PEG or PLGA-PEG for forming a particle; combining the conjugate and the base component in an organic solution to form a first organic phase; and combining the first organic phase with a first aqueous solution to form a second phase; emulsifying the second phase to form an emulsion phase; and recovering particles. In various embodiments, the emulsion phase is further homogenized.

In some embodiments, the first phase includes about 5 to about 50% weight, e.g. about 1 to about 40% solids, or about 5 to about 30% solids, e.g. about 5%, 10%, 15%, and 20%, of the conjugate and the base component. In certain embodiments, the first phase includes about 5% weight of the conjugate and the base component. In various embodiments, the organic phase comprises acetonitrile, tetrahydrofuran, ethyl acetate, isopropyl alcohol, isopropyl acetate, dimethylformamide, methylene chloride, dichloromethane, chloroform, acetone, benzyl alcohol, TWEEN® 80, SPAN® 80, or a combination thereof. In some embodiments, the organic phase includes benzyl alcohol, ethyl acetate, or a combination thereof.

In various embodiments, the aqueous solution includes water, sodium cholate, ethyl acetate, or benzyl alcohol. In various embodiments, a surfactant is added into the first phase, the second phase, or both. A surfactant, in some instances, can act as an emulsifier or a stabilizer for a composition disclosed herein. A suitable surfactant can be a cationic surfactant, an anionic surfactant, or a nonionic surfactant. In some embodiments, a surfactant suitable for making a composition described herein includes sorbitan fatty acid esters, polyoxyethylene sorbitan fatty acid esters and polyoxyethylene stearates. Examples of such fatty acid ester nonionic surfactants are the TWEEN® 80, SPAN® 80, and MYJ® surfactants from ICI. SPAN® surfactants include C₁₂-C₁₈ sorbitan monoesters. TWEEN® surfactants include poly(ethylene oxide) C₁₂-C₁₈ sorbitan monoesters. MYJ® surfactants include poly(ethylene oxide) stearates. In certain embodiments, the aqueous solution also comprises a surfactant (e.g., an emulsifier), including a polysorbate. For example, the aqueous solution can include polysorbate 80. In some embodiments, a suitable surfactant includes a lipid-based surfactant. For example, the composition can include 1,2-dihexanoyl-sn-glycero-3-phosphocholine, 1,2-diheptanoyl-sn-glycero-3-phosphocholine, PEGlyated 1,2-distearoyl-sn-glycero-3-phosphoethanolamine (including PEG5000-DSPE), PEGlyated 1,2-dioleoyl-sn-glycero-3-phosphoethanolamine (including 1,2-dioleoyl-sn-glycero-3-phosphoethanolamine-N-[methoxy(polyethylene glycol)-5000] (ammonium salt)).

Emulsifying the second phase to form an emulsion phase may be performed in one or two emulsification steps. For example, a primary emulsion may be prepared, and then emulsified to form a fine emulsion. The primary emulsion can be formed, for example, using simple mixing, a high pressure homogenizer, probe sonicator, stir bar, or a rotor stator homogenizer. The primary emulsion may be formed into a fine emulsion through the use of e.g. a probe sonicator or a high pressure homogenizer, e.g. by pass(es) through a homogenizer. For example, when a high pressure homogenizer is used, the pressure used may be about 4000 to about 8000 psi, about 4000 to about 5000 psi, or 0.4000 or 5000 psi.

Either solvent evaporation or dilution may be needed to complete the extraction of the solvent and solidify the particles. For better control over the kinetics of extraction and a more scalable process, a solvent dilution via aqueous quench may be used. For example, the emulsion can be diluted into cold water to a concentration sufficient to dissolve all of the organic solvent to form a quenched phase. Quenching may be performed at least partially at a temperature of about 5° C. or less. For example, water used in the quenching may be at a temperature that is less that room temperature (e.g. about 0 to about 10° C., or about 0 to about 5° C.).

In various embodiments, the particles are recovered by filtration. For example, ultrafiltration membranes can be used. Exemplary filtration may be performed using a tangential flow filtration system. For example, by using a membrane with a pore size suitable to retain nanoparticles while allowing solutes, micelles, and organic solvent to pass, nanoparticles can be selectively separated. Exemplary membranes with molecular weight cut-offs of about 300-500 kDa (−5-25 nm) may be used.

In various embodiments, the particles are freeze-dried or lyophilized, in some instances, to extend their shelf life. In some embodiments, the composition also includes a lyoprotectant. In certain embodiments, a lyoprotectant is selected from a sugar, a polyalcohol, or a derivative thereof. In particular embodiments, a lyoprotectant is selected from a monosaccharide, a disaccharide, or a mixture thereof. For example, a lyoprotectant can be sucrose, lactulose, trehalose, lactose, glucose, maltose, mannitol, cellobiose, or a mixture thereof.

Methods of making particles containing one or more conjugates are provided. The particles can be polymeric particles, lipid particles, or combinations thereof. The various methods described herein can be adjusted to control the size and composition of the particles, e.g. some methods are best suited for preparing microparticles while others are better suited for preparing nanoparticles. The selection of a method for preparing particles having the descried characteristics can be performed by the skilled artisan without undue experimentation.

i. Polymeric Particles

Methods of making polymeric particles are known in the art. Polymeric particles can be prepared using any suitable method known in the art. Common microencapsulation techniques include, but are not limited to, spray drying, interfacial polymerization, hot melt encapsulation, phase separation encapsulation (spontaneous emulsion microencapsulation, solvent evaporation microencapsulation, and solvent removal microencapsulation), coacervation, low temperature microsphere formation, and phase inversion nanoencapsulation (PIN). A brief summary of these methods is presented below.

### 1. Spray Drying

Methods for forming polymeric particles using spray drying techniques are described in U.S. Pat. No. 6,620,617. In this method, the polymer is dissolved in an organic solvent such as methylene chloride or in water. A known amount of one or more conjugates or additional active agents to be incorporated in the particles is suspended (in the case of an insoluble active agent) or co-dissolved (in the case of a soluble active agent) in the polymer solution. The solution or dispersion is pumped through a micronizing nozzle driven by a flow of compressed gas, and the resulting aerosol is suspended in a heated cyclone of air, allowing the solvent to evaporate from the microdroplets, forming particles. Microspheres/nanospheres ranging between 0.1 10 microns can be obtained using this method.

### 2. Interfacial Polymerization

Interfacial polymerization can also be used to encapsulate one or more conjugates and/or active agents. Using this method, a monomer and the conjugates or active agent(s) are dissolved in a solvent. A second monomer is dissolved in a second solvent (typically aqueous) which is immiscible with the first. An emulsion is formed by suspending the first solution through stirring in the second solution. Once the emulsion is stabilized, an initiator is added to the aqueous phase causing interfacial polymerization at the interface of each droplet of emulsion.

### 3. Hot Melt Microencapsulation

Microspheres can be formed from polymers such as polyesters and polyanhydrides using hot melt microencapsulation methods as described in Mathiowitz et al., Reactive Polymers, 6:275 (1987). In this method, the use of polymers with molecular weights between 3,000-75,000 daltons is preferred. In this method, the polymer first is melted and then mixed with the solid particles of one or more active agents to be incorporated that have been sieved to less than 50 microns. The mixture is suspended in a non-miscible solvent (like silicon oil), and, with continuous stirring, heated to 5° C. above the melting point of the polymer. Once the emulsion is stabilized, it is cooled until the polymer particles solidify. The resulting microspheres are washed by decanting with petroleum ether to produce a free flowing powder.

### 4. Phase Separation Microencapsulation

In phase separation microencapsulation techniques, a polymer solution is stirred, optionally in the presence of one or more active agents to be encapsulated. While continuing to uniformly suspend the material through stirring, a nonsolvent for the polymer is slowly added to the solution to decrease the polymer's solubility. Depending on the solubility of the polymer in the solvent and nonsolvent, the polymer either precipitates or phase separates into a polymer rich and a polymer poor phase. Under proper conditions, the polymer in the polymer rich phase will migrate to the interface with the continuous phase, encapsulating the active agent(s) in a droplet with an outer polymer shell.

a. Spontaneous Emulsion Microencapsulation

Spontaneous emulsification involves solidifying emulsified liquid polymer droplets formed above by changing temperature, evaporating solvent, or adding chemical cross-linking agents. The physical and chemical properties of the encapsulant, as well as the properties of the one or more active agents optionally incorporated into the nascent particles, dictates suitable methods of encapsulation. Factors such as hydrophobicity, molecular weight, chemical stability, and thermal stability affect encapsulation.

b. Solvent Evaporation Microencapsulation

Methods for forming microspheres using solvent evaporation techniques are described in Mathiowitz et al., J. Scanning Microscopy, 4:329 (1990); Beck et al., Fertil. Steril., 31:545 (1979); Beck et al., Am. J. Obstet. Gynecol. 135(3) (1979); Benita et al., J. Pharm. Sci., 73:1721 (1984); and U.S. Pat. No. 3,960,757. The polymer is dissolved in a volatile organic solvent, such as methylene chloride. One or more active agents to be incorporated are optionally added to the solution, and the mixture is suspended in an aqueous solution that contains a surface active agent such as poly(vinyl alcohol). The resulting emulsion is stirred until most of the organic solvent evaporated, leaving solid microparticles/nanoparticles. This method is useful for relatively stable polymers like polyesters and polystyrene.

c. Solvent Removal Microencapsulation

The solvent removal microencapsulation technique is primarily designed for polyanhydrides and is described, for example, in WO 93/21906. In this method, the substance to be incorporated is dispersed or dissolved in a solution of the selected polymer in a volatile organic solvent, such as methylene chloride. This mixture is suspended by stirring in an organic oil, such as silicon oil, to form an emulsion. Microspheres that range between 1-300 microns can be obtained by this procedure. Substances which can be incorporated in the microspheres include pharmaceuticals, pesticides, nutrients, imaging agents, and metal compounds.

### 5. Coacervation

Encapsulation procedures for various substances using coacervation techniques are known in the art, for example, in GB-B-929 406; GB-B-929 40 1; and U.S. Pat. Nos. 3,266,987, 4,794,000, and 4,460,563. Coacervation involves the separation of a macromolecular solution into two immiscible liquid phases. One phase is a dense coacervate phase, which contains a high concentration of the polymer encapsulant (and optionally one or more active agents), while the second phase contains a low concentration of the polymer. Within the dense coacervate phase, the polymer encapsulant forms nanoscale or microscale droplets. Coacervation may be induced by a temperature change, addition of a non-solvent or addition of a micro-salt (simple coacervation), or by the addition of another polymer thereby forming an interpolymer complex (complex coacervation).

### 6. Low Temperature Casting of Microspheres

Methods for very low temperature casting of controlled release particles are described in U.S. Pat. No. 5,019,400. In this method, a polymer is dissolved in a solvent optionally with one or more dissolved or dispersed active agents. The mixture is then atomized into a vessel containing a liquid non solvent at a temperature below the freezing point of the polymer substance solution which freezes the polymer droplets. As the droplets and non solvent for the polymer are warmed, the solvent in the droplets thaws and is extracted into the non solvent, resulting in the hardening of the microspheres.

### 7. Phase Inversion Nanoencapsulation (PIN)

Nanoparticles can also be formed using the phase inversion nanoencapsulation (PIN) method, wherein a polymer is dissolved in a “good” solvent, fine particles of a substance to be incorporated, such as a drug, are mixed or dissolved in the polymer solution, and the mixture is poured into a strong non solvent for the polymer, to spontaneously produce, under favorable conditions, polymeric microspheres, wherein the polymer is either coated with the particles or the particles are dispersed in the polymer. See, e.g., U.S. Pat. No. 6,143,211. The method can be used to produce monodisperse populations of nanoparticles and microparticles in a wide range of sizes, including, for example, about 100 nanometers to about 10 microns. [000214] Advantageously, an emulsion need not be formed prior to precipitation. The process can be used to form microspheres from thermoplastic polymers.

### 8. Emulsion Methods

In some embodiments, a nanoparticle is prepared using an emulsion solvent evaporation method. For example, a polymeric material is dissolved in a water immiscible organic solvent and mixed with a drug solution or a combination of drug solutions. In some embodiments a solution of a therapeutic, prophylactic, or diagnostic agent to be encapsulated is mixed with the polymer solution. The polymer can be, but is not limited to, one or more of the following: PLA, PGA, PCL, their copolymers, polyacrylates, the aforementioned PEGylated polymers. The drug molecules can include one or more conjugates as described above and one or more additional active agents. The water immiscible organic solvent, can be, but is not limited to, one or more of the following: chloroform, dichloromethane, and acyl acetate. The drug can be dissolved in, but is not limited to, one or more of the following: acetone, ethanol, methanol, isopropyl alcohol, acetonitrile and Dimethyl sulfoxide (DMSO).

An aqueous solution is added into the resulting polymer solution to yield emulsion solution by emulsification. The emulsification technique can be, but not limited to, probe sonication or homogenization through a homogenizer.

### 9. Nanoprecipitation

In another embodiment, a conjugate containing nanoparticle is prepared using nanoprecipitation methods or microfluidic devices. The conjugate containing polymeric material is mixed with a drug or drug combinations in a water miscible organic solvent, optionally containing additional polymers. The additional polymer can be, but is not limited to, one or more of the following: PLA, PGA, PCL, their copolymers, polyacrylates, the aforementioned PEGylated polymers. The water miscible organic solvent, can be, but is not limited to, one or more of the following: acetone, ethanol, methanol, isopropyl alcohol, acetonitrile and dimethyl sulfoxide (DMSO). The resulting mixture solution is then added to a polymer non-solvent, such as an aqueous solution, to yield nanoparticle solution.

### 10. Microfluidics

Methods of making nanoparticles using microfluidics are known in the art. Suitable methods include those described in U.S. Patent Application Publication No. 2010/0022680 A1. In general, the microfluidic device comprises at least two channels that converge into a mixing apparatus. The channels are typically formed by lithography, etching, embossing, or molding of a polymeric surface. A source of fluid is attached to each channel, and the application of pressure to the source causes the flow of the fluid in the channel. The pressure may be applied by a syringe, a pump, and/or gravity. The inlet streams of solutions with polymer, targeting moieties, lipids, drug, payload, etc. converge and mix, and the resulting mixture is combined with a polymer non-solvent solution to form the nanoparticles having the desired size and density of moieties on the surface. By varying the pressure and flow rate in the inlet channels and the nature and composition of the fluid sources nanoparticles can be produced having reproducible size and structure.

ii. Lipid Particles

Methods of making lipid particles are known in the art. Lipid particles can be lipid micelles, liposomes, or solid lipid particles prepared using any suitable method known in the art. Common techniques for created lipid particles encapsulating an active agent include, but are not limited to high pressure homogenization techniques, supercritical fluid methods, emulsion methods, solvent diffusion methods, and spray drying. A brief summary of these methods is presented below.

### 1. High Pressure Homogenization (HPH) Methods

High pressure homogenization is a reliable and powerful technique, which is used for the production of smaller lipid particles with narrow size distributions, including lipid micelles, liposomes, and solid lipid particles. High pressure homogenizers push a liquid with high pressure (100-2000 bar) through a narrow gap (in the range of a few microns). The fluid can contain lipids that are liquid at room temperature or a melt of lipids that are solid at room temperature. The fluid accelerates on a very short distance to very high velocity (over 1000 Km/h). This creates high shear stress and cavitation forces that disrupt the particles, generally down to the submicron range. Generally 5-10% lipid content is used but up to 40% lipid content has also been investigated.

Two approaches of HPH are hot homogenization and cold homogenization, work on the same concept of mixing the drug in bulk of lipid solution or melt.

a. Hot Homogenization:

Hot homogenization is carried out at temperatures above the melting point of the lipid and can therefore be regarded as the homogenization of an emulsion. A pre-emulsion of the drug loaded lipid melt and the aqueous emulsifier phase is obtained by a high-shear mixing. HPH of the pre-emulsion is carried out at temperatures above the melting point of the lipid. A number of parameters, including the temperature, pressure, and number of cycles, can be adjusted to produce lipid particles with the desired size. In general, higher temperatures result in lower particle sizes due to the decreased viscosity of the inner phase. However, high temperatures increase the degradation rate of the drug and the carrier. Increasing the homogenization pressure or the number of cycles often results in an increase of the particle size due to high kinetic energy of the particles.

b. Cold Homogenization

Cold homogenization has been developed as an alternative to hot homogenization. Cold homogenization does not suffer from problems such as temperature-induced drug degradation or drug distribution into the aqueous phase during homogenization. The cold homogenization is particularly useful for solid lipid particles, but can be applied with slight modifications to produce liposomes and lipid micelles. In this technique the drug containing lipid melt is cooled, the solid lipid ground to lipid microparticles and these lipid microparticles are dispersed in a cold surfactant solution yielding a pre-suspension. The pre-suspension is homogenized at or below room temperature, where the gravitation force is strong enough to break the lipid microparticles directly to solid lipid nanoparticles.

### 2. Ultrasonication/high Speed Homogenization Methods

Lipid particles, including lipid micelles, liposomes, and solid lipid particles, can be prepared by ultrasonication/high speed homogenization. The combination of both ultrasonication and high speed homogenization is particularly useful for the production of smaller lipid particles. Liposomes are formed in the size range from 10 nm to 200 nm, preferably 50 nm to 100 nm, by this process.

### 3. Solvent Evaporation Methods

Lipid particles can be prepared by solvent evaporation approaches. The lipophilic material is dissolved in a water-immiscible organic solvent (e.g. cyclohexane) that is emulsified in an aqueous phase. Upon evaporation of the solvent, nanoparticles dispersion is formed by precipitation of the lipid in the aqueous medium. Parameters such as temperature, pressure, choices of solvents can be used to control particle size and distribution. Solvent evaporation rate can be adjusted through increased/reduced pressure or increased/reduced temperature.

### 4. Solvent Emulsification-diffusion Methods

Lipid particles can be prepared by solvent emulsification-diffusion methods. The lipid is first dissolved in an organic phase, such as ethanol and acetone. An acidic aqueous phase is used to adjust the zeta potential to induce lipid coacervation. The continuous flow mode allows the continuous diffusion of water and alcohol, reducing lipid solubility, which causes thermodynamic instability and generates liposomes

### 5. Supercritical Fluid Methods

Lipid particles, including liposomes and solid lipid particles, can be prepared from supercritical fluid methods. Supercritical fluid approaches have the advantage of replacing or reducing the amount of the organic solvents used in other preparation methods. The lipids, active agents to be encapsulated, and excipients can be solvated at high pressure in a supercritical solvent. The supercritical solvent is most commonly CO₂, although other supercritical solvents are known in the art. To increase solubility of the lipid, a small amount of co-solvent can be used. Ethanol is a common co-solvent, although other small organic solvents that are generally regarded as safe for formulations can be used. The lipid particles, lipid micelles, liposomes, or solid lipid particles can be obtained by expansion of the supercritical solution or by injection into a non-solvent aqueous phase. The particle formation and size distribution can be controlled by adjusting the supercritical solvent, co-solvent, non-solvent, temperatures, pressures, etc.

### 6. Microemulsion Based Methods

Microemulsion based methods for making lipid particles are known in the art. These methods are based upon the dilution of a multiphase, usually two-phase, system. Emulsion methods for the production of lipid particles generally involve the formation of a water-in-oil emulsion through the addition of a small amount of aqueous media to a larger volume of immiscible organic solution containing the lipid. The mixture is agitated to disperse the aqueous media as tiny droplets throughout the organic solvent and the lipid aligns itself into a monolayer at the boundary between the organic and aqueous phases. The size of the droplets is controlled by pressure, temperature, the agitation applied and the amount of lipid present.

The water-in-oil emulsion can be transformed into a liposomal suspension through the formation of a double emulsion. In a double emulsion, the organic solution containing the water droplets is added to a large volume of aqueous media and agitated, producing a water-in-oil-in-water emulsion. The size and type of lipid particle formed can be controlled by the choice of and amount of lipid, temperature, pressure, co-surfactants, solvents, etc.

### 7. Spray Drying Methods

Spray drying methods similar to those described above for making polymeric particle can be employed to create solid lipid particles. This works best for lipid with a melting point above 70° C.

### VI. Methods of Using the Conjugates and Nanoparticles

The formulations can be administered to treat any proliferative disease, metabolic disease, infectious disease, or cancer, as appropriate. The formulations can be used for immunization. Formulations are administered by injection, orally, or topically, typically to a mucosal surface (lung, nasal, oral, buccal, sublingual, vaginally, rectally) or to the eye (intraocularly or transocularly). The formulations conjugate containing particles described herein can be used for the selective tissue delivery of a therapeutic, prophylactic, or diagnostic agent to an individual or patient in need thereof. Dosage regimens may be adjusted to provide the optimum desired response (e.g., a therapeutic or prophylactic response). For example, a single bolus may be administered, several divided doses may be administered over time or the dose may be proportionally reduced or increased as indicated by the exigencies of the therapeutic situation. Dosage unit form as used herein refers to physically discrete units suited as unitary dosages for the mammalian subjects to be treated; each unit containing a predetermined quantity of active compound calculated to produce the desired therapeutic.

In various embodiments, a conjugate contained within a particle is released in a controlled manner. The release can be in vitro or in vivo. For example, particles can be subject to a release test under certain conditions, including those specified in the U.S. Pharmacopeia and variations thereof.

In various embodiments, less than about 90%, less than about 80%, less than about 70%, less than about 60%, less than about 50%, less than about 40%, less than about 30%, less than about 20% of the conjugate contained within particles is released in the first hour after the particles are exposed to the conditions of a release test. In some embodiments, less that about 90%, less than about 80%, less than about 70%, less than about 60%, or less than about 50% of the conjugate contained within particles is released in the first hour after the particles are exposed to the conditions of a release test. In certain embodiments, less than about 50% of the conjugate contained within particles is released in the first hour after the particles are exposed to the conditions of a release test.

With respect to a conjugate being released in vivo, for instance, the conjugate contained within a particle administered to a subject may be protected from a subject's body, and the body may also be isolated from the conjugate until the conjugate is released from the particle.

Thus, in some embodiments, the conjugate may be substantially contained within the particle until the particle is delivered into the body of a subject. For example, less than about 90%, less than about 80%, less than about 70%, less than about 60%, less than about 50%, less than about 40%, less than about 30%, less than about 20%, less than about 15%, less than about 10%, less than about 5%, or less than about 1% of the total conjugate is released from the particle prior to the particle being delivered into the body, for example, a treatment site, of a subject. In some embodiments, the conjugate may be released over an extended period of time or by bursts (e.g., amounts of the conjugate are released in a short period of time, followed by a periods of time where substantially no conjugate is released). For example, the conjugate can be released over 6 hours, 12 hours, 24 hours, or 48 hours. In certain embodiments, the conjugate is released over one week or one month.

### Exemplary Embodiments

### Exemplary Embodiment 1

### Synthesis of a Folate-Platinum(IV) Conjugate

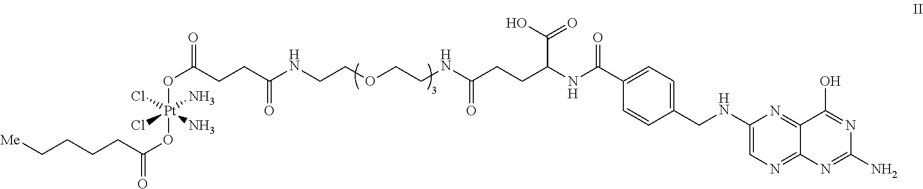

The folate-platinum(IV) targeted conjugate of Formula II (above) is prepared according to the following reaction scheme or modifications thereof.

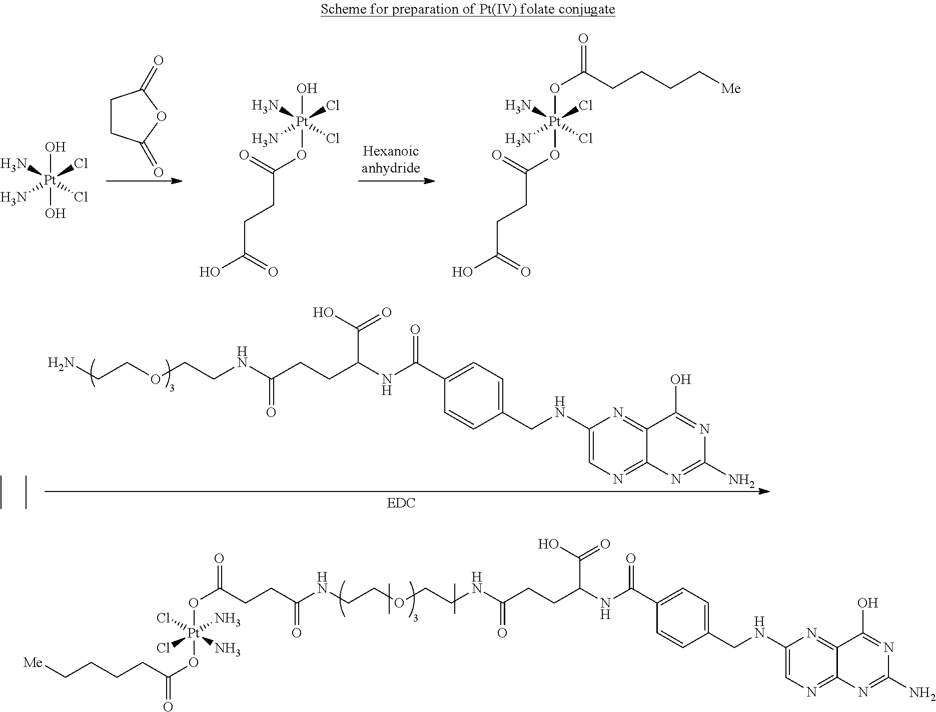

Dihydroxycisplatin(IV) is reacted with succinic anhydride in DMSO at ambient temperature. The resulting isolated succinate is reacted with hexanoic anhydride in N,N,-dimethylformatmide at ambient temperature to provide the monosuccinate monohexanoate cisplatin(IV). Coupling of this intermediate with the folic acid derived amine described in the literature provides the folate-Pt(IV) conjugate shown. The conjugate is formulated into nanoparticles as described herein.

### Exemplary Embodiment 2

### Synthesis of a PSMA-Cabazitaxel Conjugate

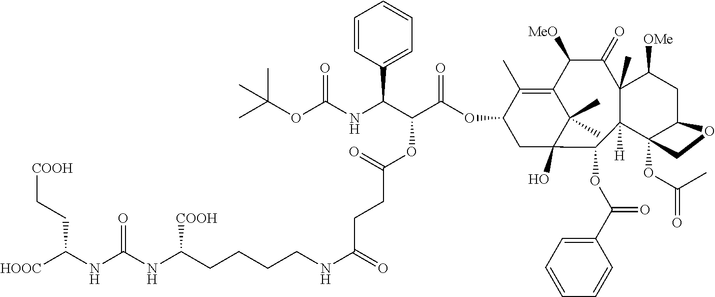

The PSMA-cabazitaxel targeted conjugate of Formula III (above) is prepared according to the following reaction scheme or slight modifications thereof.

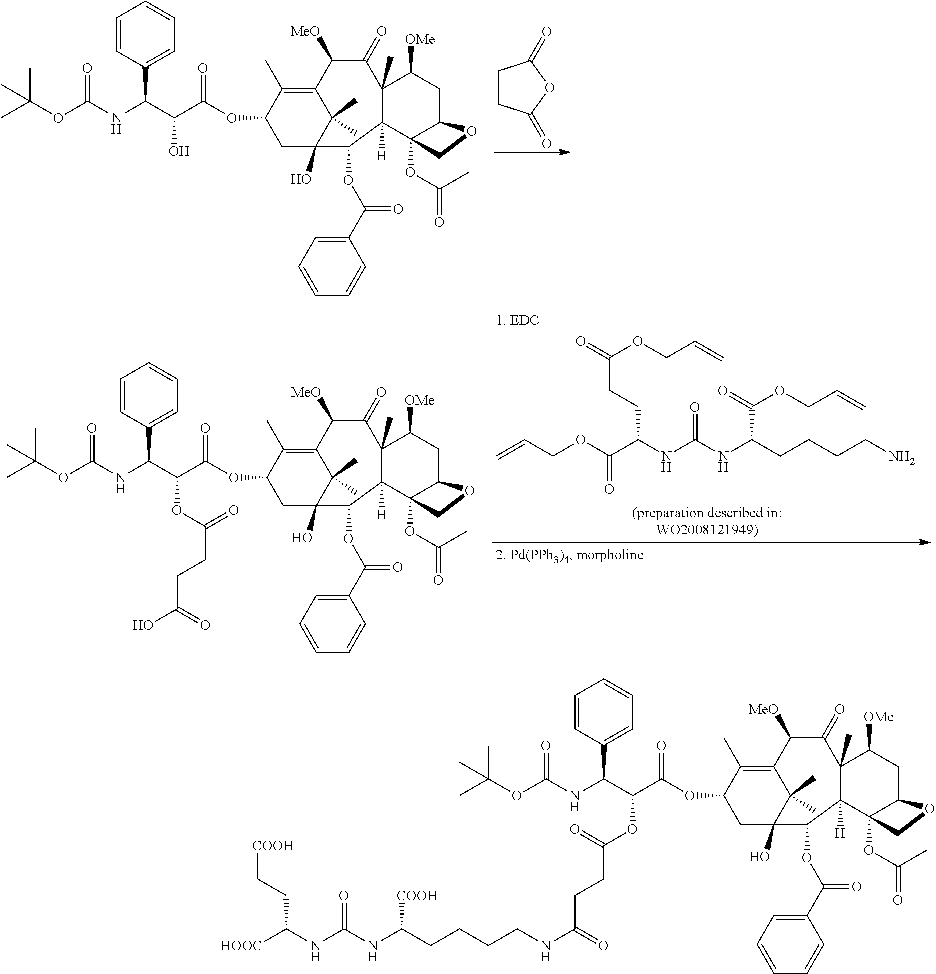

Cabazitaxel is reacted with succinic anhydride in dichloromethane with a catalytic amount of N,N-dimethyl-4-aminopyridine at ambient temperature. The resulting succinate is reacted with the amine described in the patent literature using carbodiimide coupling conditions in chlorinated solvent or N,N-dimethylformamide to provide a protected version of the conjugate. Deprotection of this conjugate using tetrakistrphenylphosphine palladium(0) and morpholine provides the desired cabazitaxel-PSMA ligand conjugate.

The conjugate is formulated in nanoparticles as described herein.

### Exemplary Embodiment 3

### Synthesis of a PSMA-Platinum(IV) Conjugate

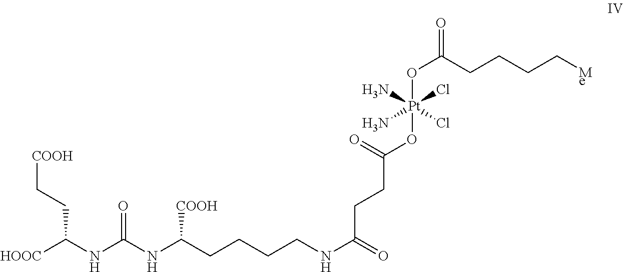

The PSMA-platinum (IV) targeted conjugate of Formula IV (above) is prepared according to the following reaction scheme.

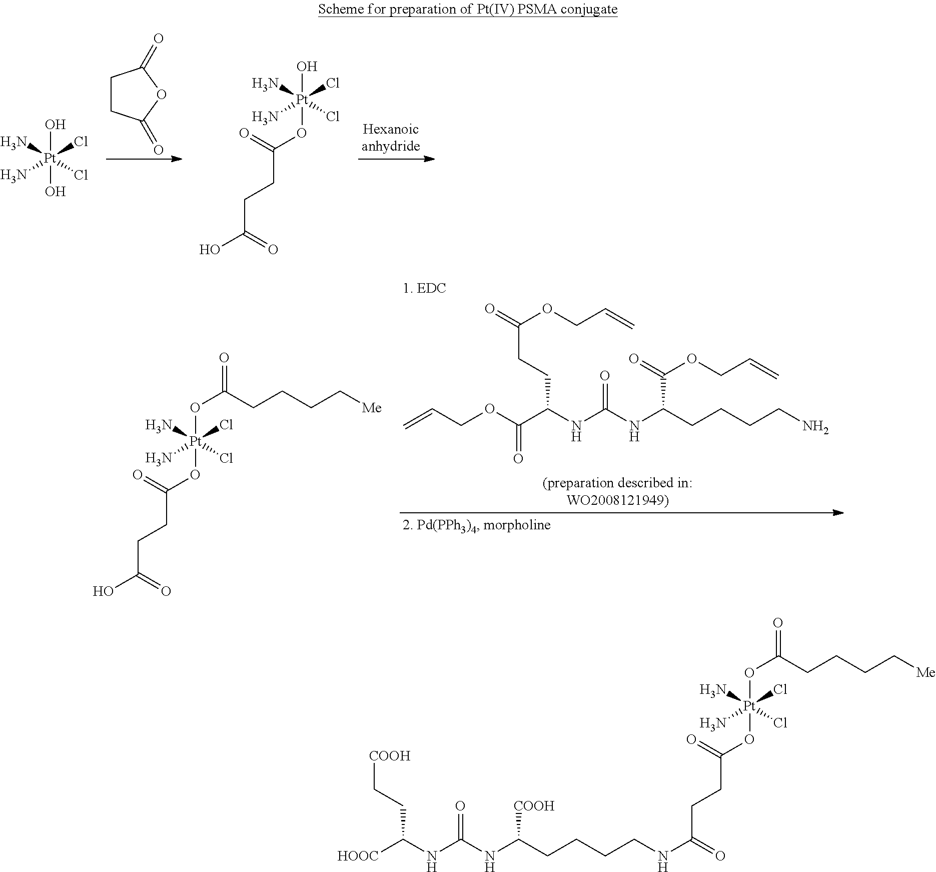

Dihydroxycisplatin(IV) is reacted with succinic anhydride in DMSO at ambient temperature. The resulting isolated succinate is reacted with hexanoic anhydride in N,N,-dimethylformatmideat ambient temperature to provide the monosuccinate monohexanoate cisplatin(IV). The resulting succinate is reacted with the amine described in the patent literature using carbodiimide coupling conditions in chlorinated solvent or N,N-dimethylformamide to provide a protected version of the conjugate. Deprotection of this conjugate using tetrakistrphenylphosphine palladium(0) and morpholine provides the desired cisplatin(IV)-PSMA ligand conjugate.

The conjugate is formulated in a nanoparticle as described herein.

### Exemplary Embodiment 4

### Synthesis of a Folate-Cabazitaxel Conjugate

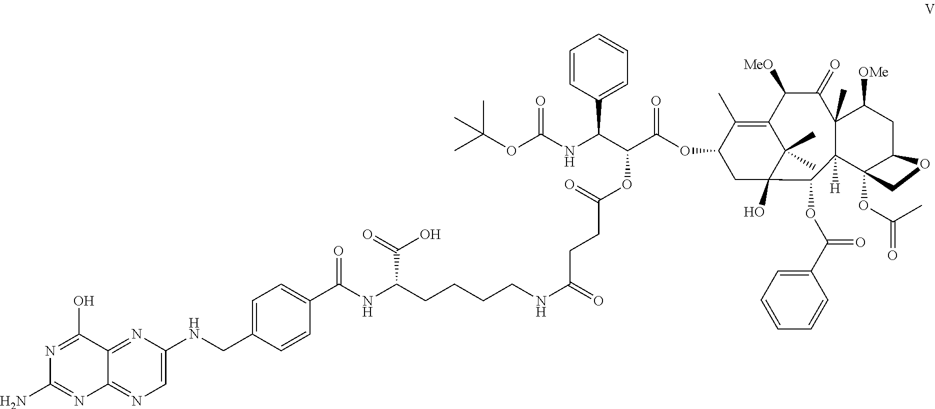

The folate-cabazitaxel targeted conjugate of Formula V (above) is prepared according to the following reaction scheme or slight modifications thereof.

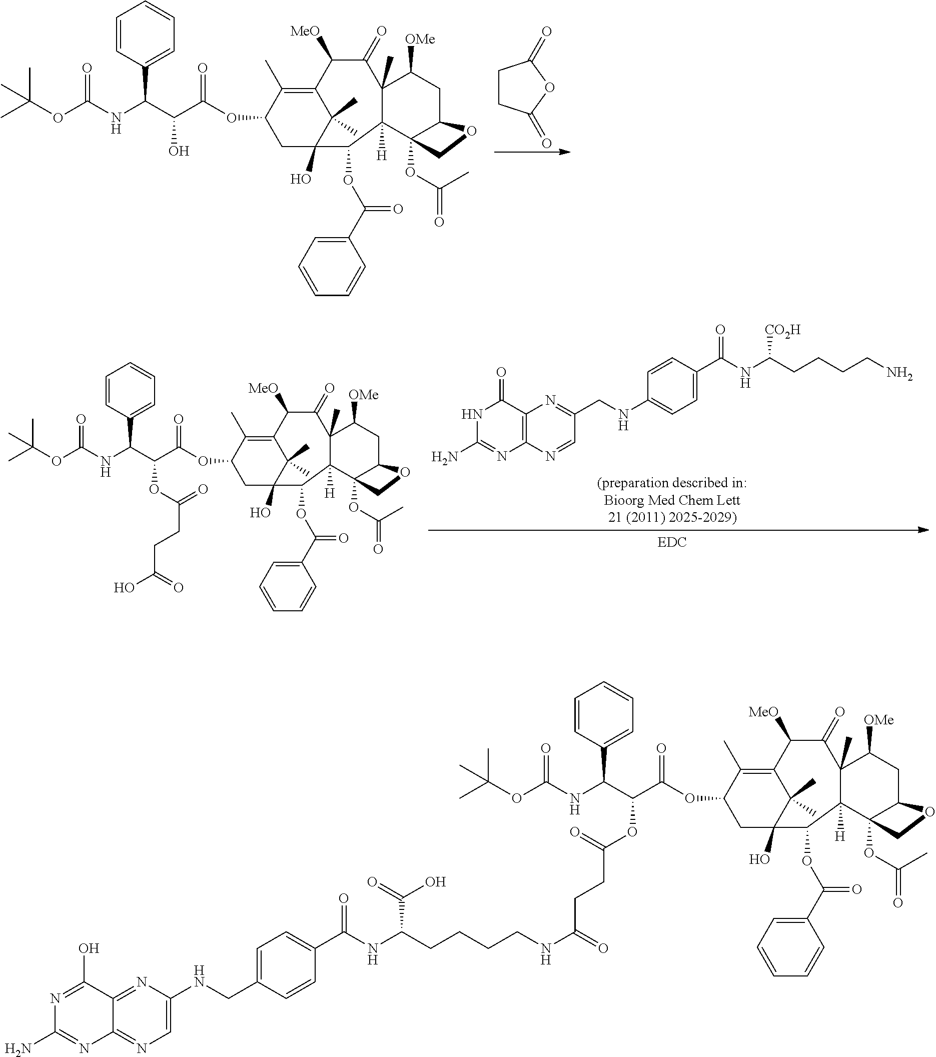

Cabazitaxel is reacted with succinic anhydride in dichloromethane with a catalytic amount of N,N-dimethyl-4-aminopyridine at ambient temperature. Coupling of this intermediate with the folic acid derived amine described in the literature provides the folate-caazitaxel conjugate shown.

The conjugate is formulated in nanoparticles as described herein.

### Exemplary Embodiment 5

### Synthesis of a PSMA-Cabazitaxel Conjugate

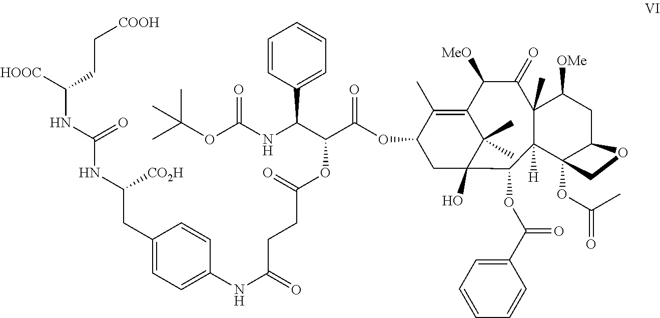

The PSMA-cabazitaxel targeted drug conjugate of Formula VI is prepared according to the following synthetic procedure or modifications thereof:

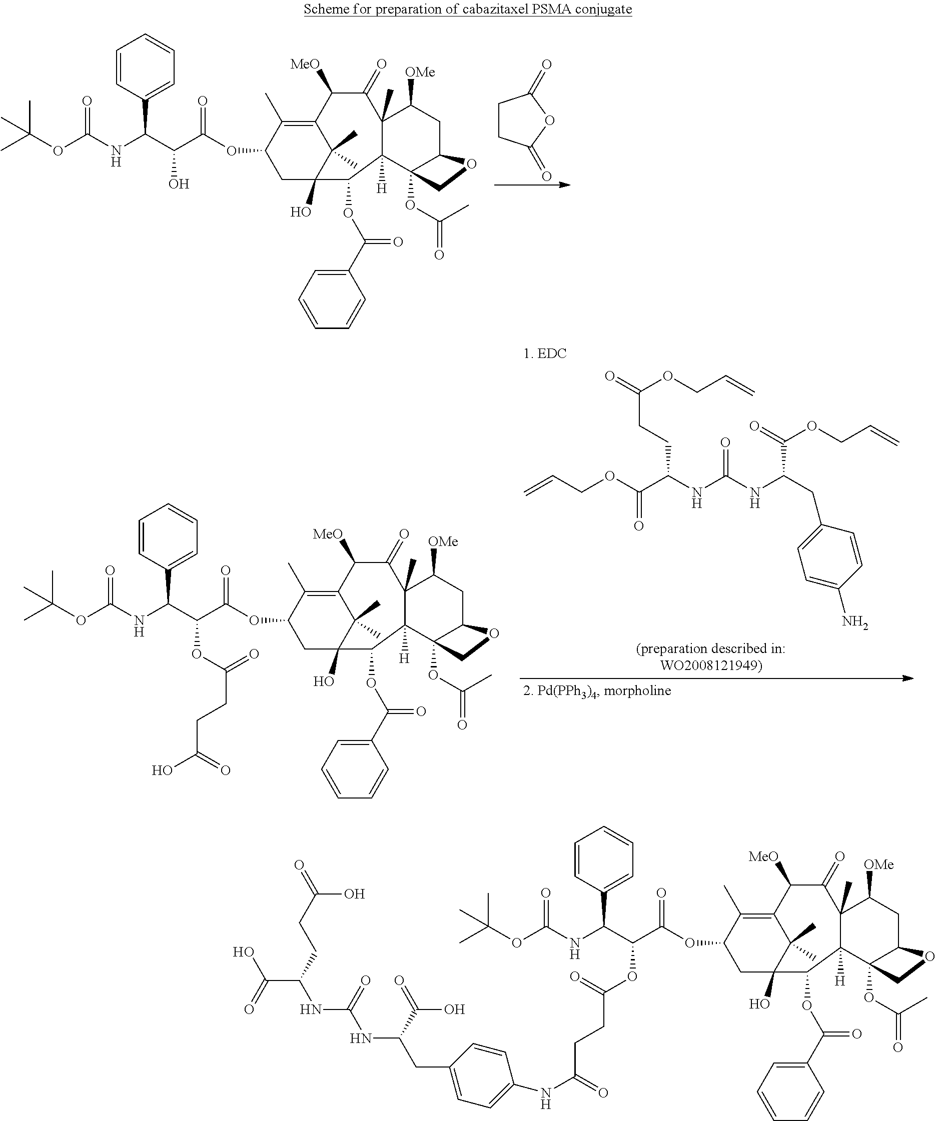

Cabazitaxel is reacted with succinic anhydride in dichloromethane with a catalytic amount of N,N-dimethyl-4-aminopyridine at ambient temperature. The resulting succinate is reacted with the amine described in the patent literature using carbodiimide coupling conditions in chlorinated solvent or N,N-dimethylformamide to provide a protected version of the conjugate. Deprotection of this conjugate using tetrakistrphenylphosphine palladium(0) and morpholine provides the desired cabazitaxel-PSMA ligand conjugate. The conjugate is formulated in nanoparticles as described herein.

### Exemplary Embodiment 6

### Synthesis of a PSMA-Cabazitaxel Conjugate

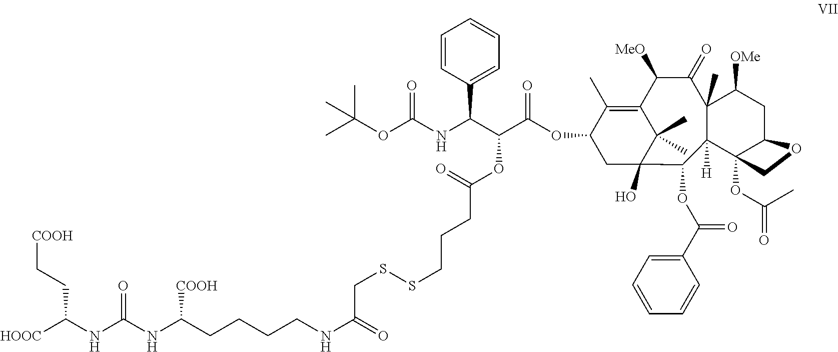

The PSMA-cabazitaxel targeted conjugate of Formula VII (above) is prepared according to the following reaction scheme or slight modifications thereof.

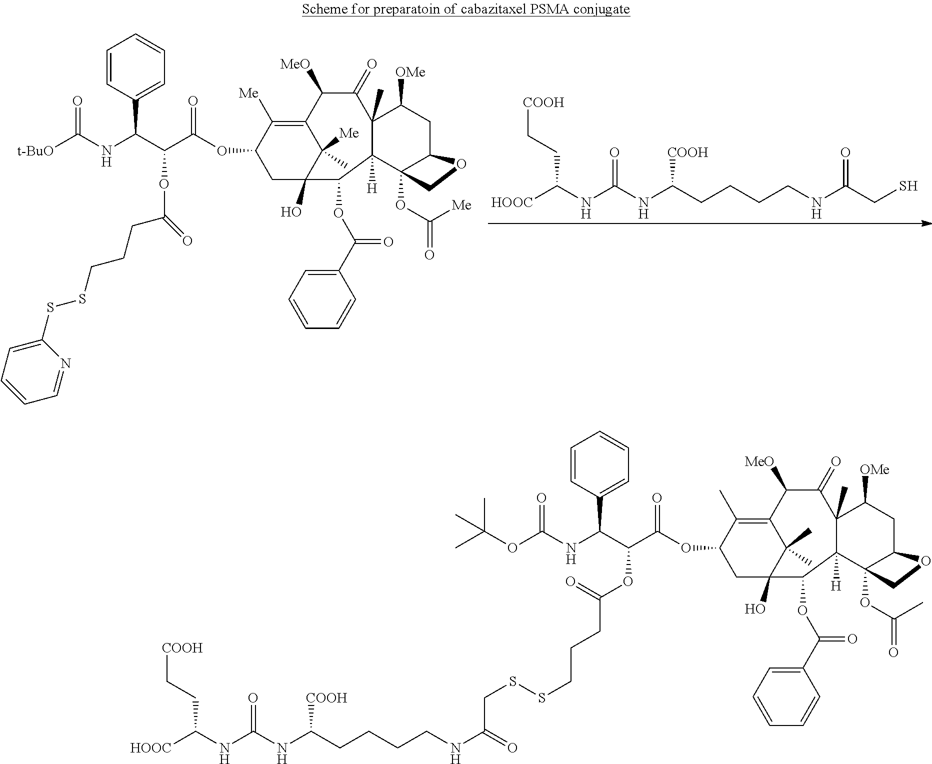

Cabazitaxel disulfide prepared in Example 1 is reacted with PSMA ligand as a thioacetamide to provide the disulfide conjugated PSMA-cabazitaxel. The conjugate is formulated in nanoparticles as described herein.

### Exemplary Embodiment 7

### Synthesis of a Folate-Pt(IV) Conjugate

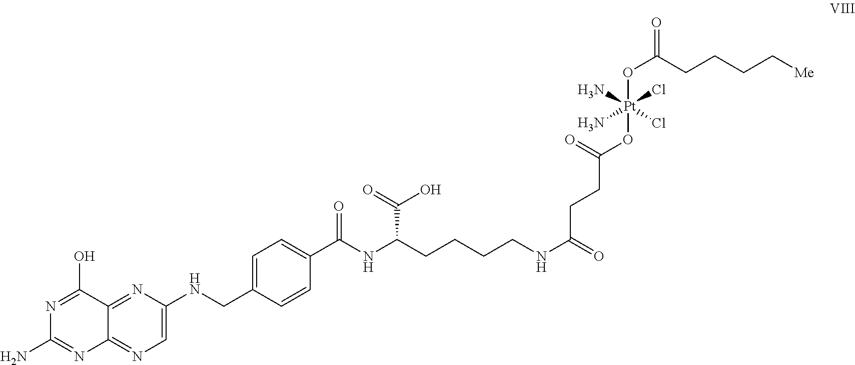

The Folate-Pt(IV) targeted conjugate of Formula VIII (above) is prepared according to the following reaction scheme or slight modifications thereof.

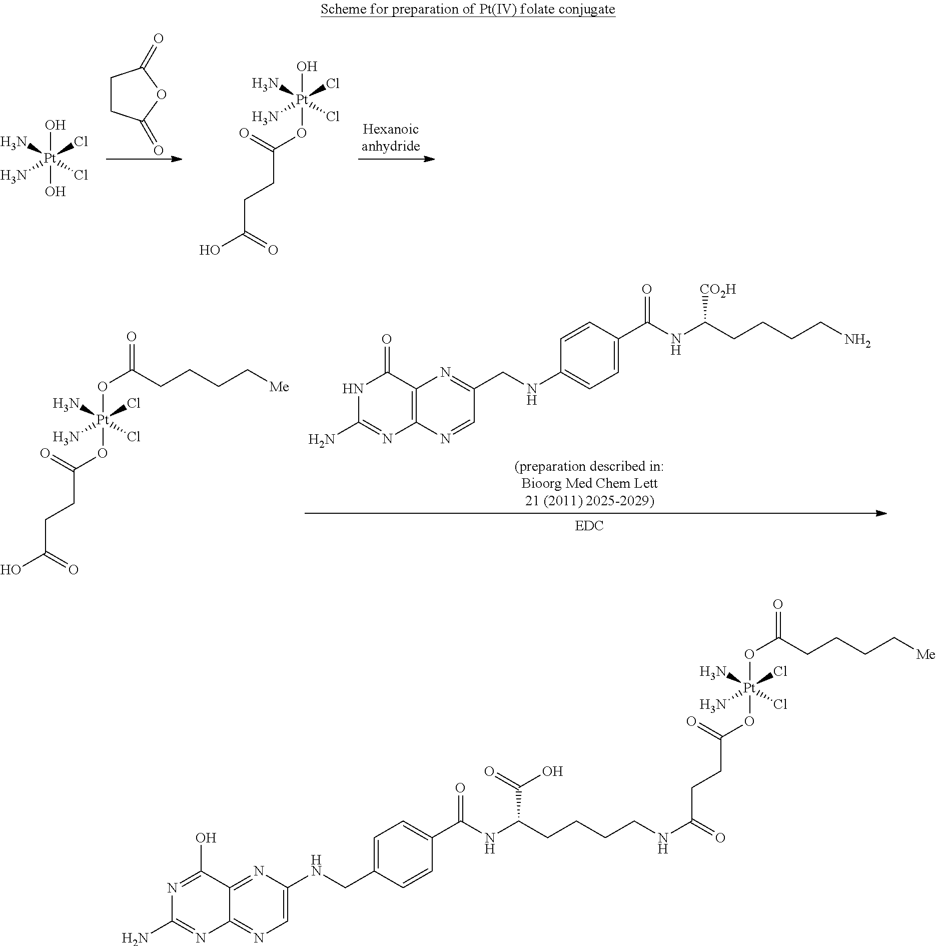

Dihydroxycisplatin(IV) is reacted with succinic anhydride in DMSO at ambient temperature. The resulting isolated succinate is reacted with hexanoic anhydride in N,N,-dimethylformatmide at ambient temperature to provide the monosuccinate monohexanoate cisplatin(IV). Coupling of this intermediate with the folic acid derived amine described in the literature provides the folate-Pt(IV) conjugate shown. The conjugate is formulated in nanoparticles as described herein.

### Exemplary Embodiment 8

### Synthesis of a Di-folate-Pt(IV) Conjugate

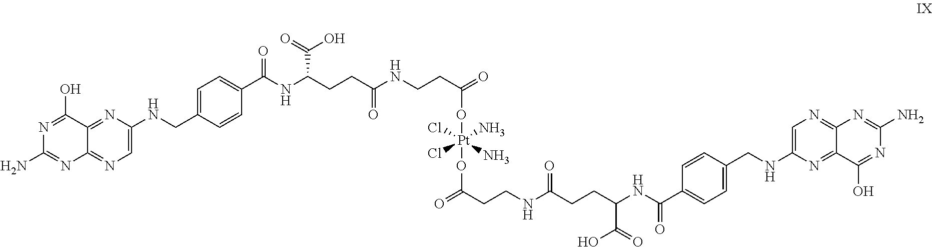

The Di-folate-Pt(IV) targeted conjugate of Formula IX is prepared according to the following reaction scheme or slight modifications thereof.

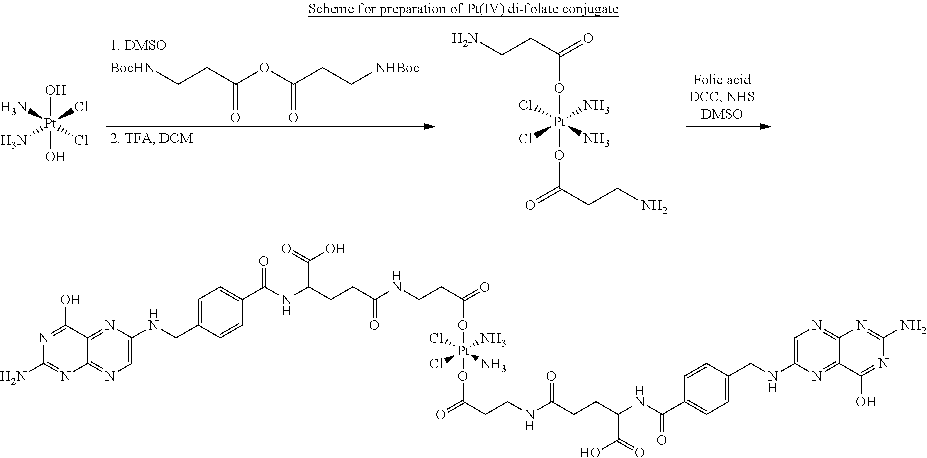

Dihydroxycisplatin(IV) is reacted with Boc-beta-alanine anhydride in DMSO at ambient temperature and the resulting product is deprotected with TFA in DCM at ambient temperature. Reaction of the resulting diamine with excess folic acid in the presence of dicyclohexylcarbodiimide, N-hydroxysuccinimide in DMSO provides the difolate-Pt(IV) conjugate. The conjugate is formulated in nanoparticles as described herein.

### Exemplary Embodiment 9

### Synthesis of a PSMA-di-Pt(IV) Conjugate

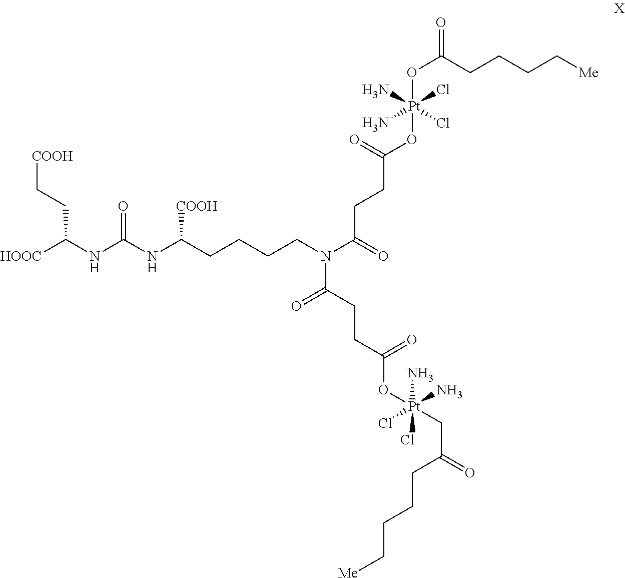

The PSMA-Di- —Pt(IV) targeted conjugate of Formula X is prepared according to the following reaction scheme or slight modifications thereof.

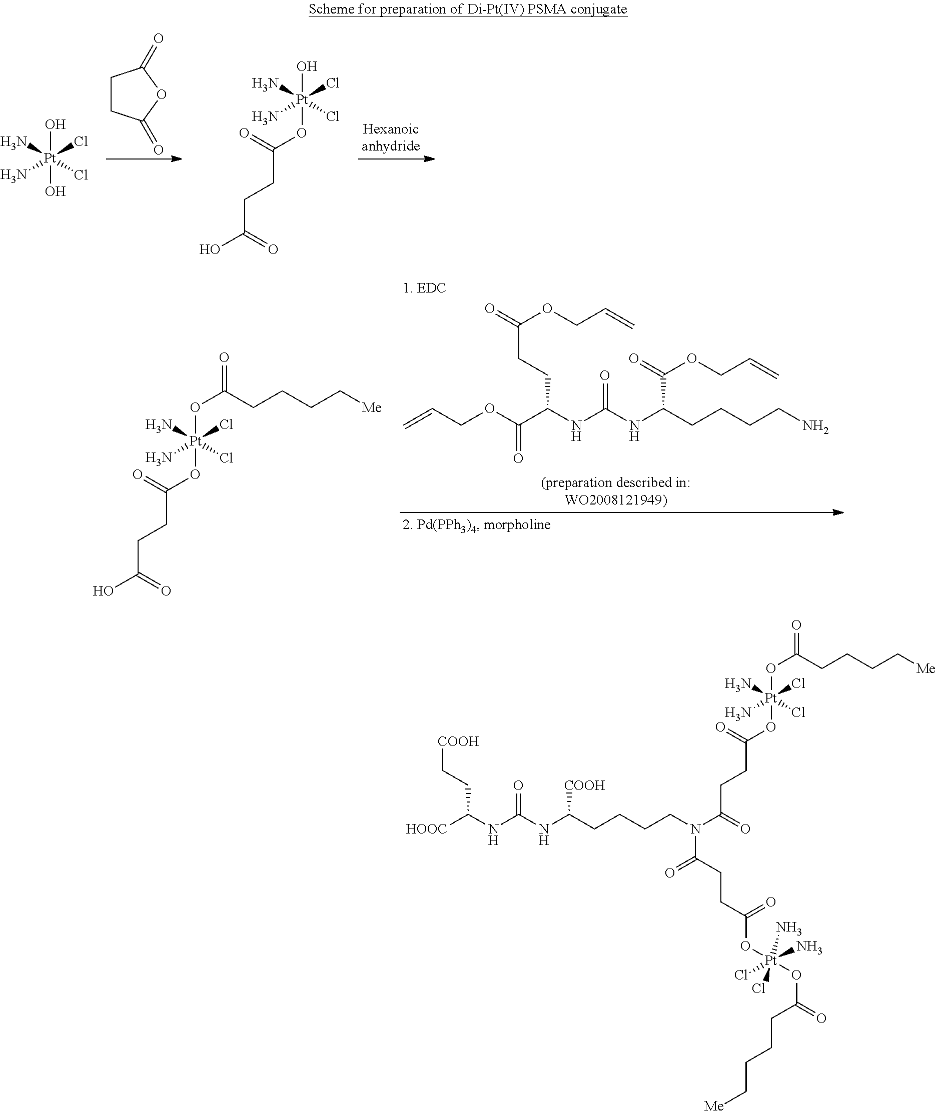

Dihydroxycisplatin(IV) is reacted with succinic anhydride in DMSO at ambient temperature. The resulting isolated succinate is reacted with hexanoic anhydride in N,N,-dimethylformatmide at ambient temperature to provide the monosuccinate monohexanoate cisplatin(IV). The resulting succinate is reacted in excess with the amine described in the patent literature using carbodiimide coupling conditions in chlorinated solvent or N,N-dimethylformamide to provide a protected version of the conjugate. Deprotection of this conjugate using tetrakistrphenylphosphine palladium(0) and morpholine provides the desired di-cisplatin(IV)-PSMA ligand conjugate. The conjugate is formulated in nanoparticles as described herein.

### EXAMPLES

### Example 1

### Synthesis of a RGD-SS-Cabazitaxel Conjugate

The RGD peptide-cabazitaxel targeted drug conjugate of Formula I was prepared according to the following synthetic procedure (Scheme II):

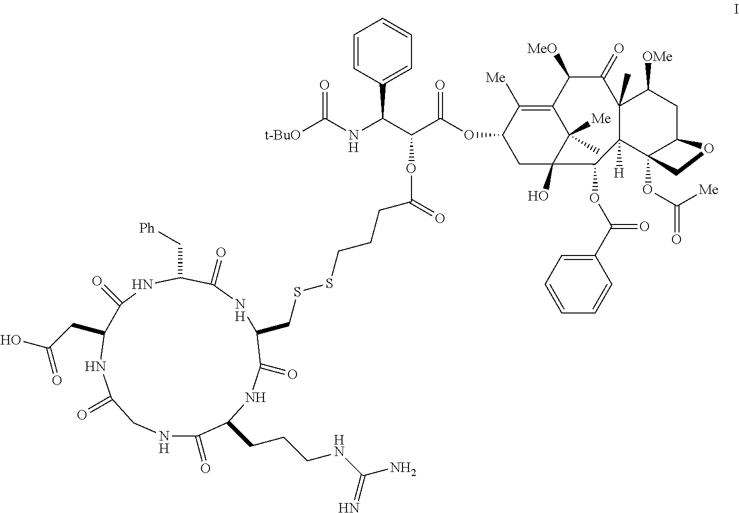

### Procedure

Step 1 Gamma-thiolactone (3 g, 29.4 mmol) was added to a 100 mL round bottom flask with a stir bar. THF (30 mL) and deionized water (20 mL) were added and the mixture was stirred at room temperature (RT). After 5 minutes (min), 5N NaOH (10 mL) was added and the resulting mixture was stirred at RT for 3 hours (h). Subsequently, the solvent was removed under vacuum at 40° C. 30 mL deionized water was then added to the crude mixture followed by concentrated HCl until pH 2 was achieved. The product was extracted three times with 30 mL ethyl acetate each time. The ethyl acetate was combined, dried over sodium sulfate and filtered. The solution was then added dropwise over the course of 1 h to a stirred mixture of 2,2′-dithiopyridine (6.5 g, 29.6 mmol) in 30 mL absolute ethanol. After the addition was complete, the reaction mixture was stirred for an additional 16 h at RT at which point the solvent was removed under vacuum at 30° C. The crude reaction mixture was purified via silica gel chromatography (2:1:0.02 heptane:ethyl acetate:acetic acid) to afford desired product in 76% yield (5.1g).

Step 2. Cabazitaxel (100 mg, 0.12 mmol), 4-(2-pyridyldithio)-butanoic acid (27 mg, 0.12 mmol), N,N′-dicyclohexylcarbodiimide (25 mg, 0.12 mmol), and 4-dimethylaminopyridine (1.5 mg, 0.012 mmol) were added to a 8 mL vial with a stir bar. Dichloromethane (2 mL) was added and the resulting solution was stirred at RT for 16 h. At this point, the reaction mixture was filtered to remove dicyclohexylurea and solvent removed under vacuum at 25° C. to afford a colorless solid. The crude material was purified via silica gel chromatography (1:1 ethyl acetate:heptane) to afford a white powder in 83% yield (104 mg). The product was analyzed by HPLC-MS (Method 1). The peak at 7.03 min affords the product parent ion of 1047 Da (M+H) (Water ZQ Micromass), which corresponds to compound of Formula I.

Step 3. Cabazitaxel butyrate pyridyldisulfide (SSPy) (18 mg, 17.2 μmol) and c(RGDfC) (10 mg, 17.2 μmol) were added to a 8 mL vial with a stir bar. 1 mL dimethylformamide (DMF) was added and the reaction mixture was stirred at RT for 16 h. The solvent was then removed under vacuum at 40° C. to afford a yellow oil, which was chased with 5 mL dichloromethane three times to afford a yellow powder (25 mg, 96% yield). The product was analyzed by HPLC-MS (Method 1). The peak at 5.20 min affords the product parent ion of 1515 Da (M+H) (Water ZQ Micromass), which corresponds to the compound of Formula I.

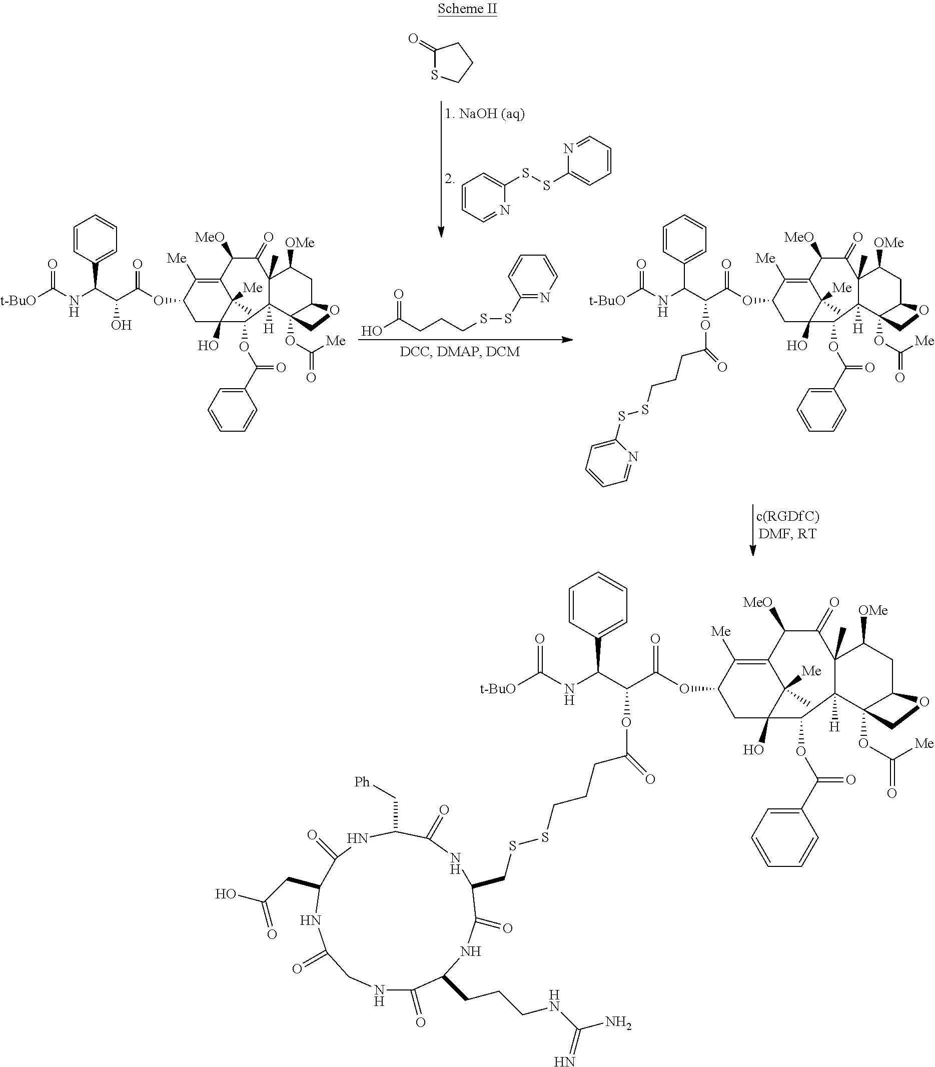

Analysis of the product by C18 Reverse Phase HPLC (Method 1)

The HPLC analysis of the RGD-SS-cabazitaxel drug conjugate was carried out on Zorbax Eclipse XDB-C18 reverse phase column (4.6×100 mm, 3.5 μm, Agilent PN: 961967-902) with a mobile phase consisting of water +0.1% TFA (solvent A) and acetonitrile +0.1% TFA (solvent B at a flow rate of the 1.5 mL/min and column temperature of 35° C. The injection volume was 10 μL and the analyte was detected using UV at 220 and 254 nm.

Gradient:

| Time (mins) | % A | % B |
|---|---|---|
| 0 | 95 | 5 |
| 6 | 5 | 95 |
| 8 | 5 | 95 |
| 8.01 | 95 | 5 |
| 10 | 95 | 5 |

### Example 2

### Synthesis of a Cabazitaxel-RGD Conjugate

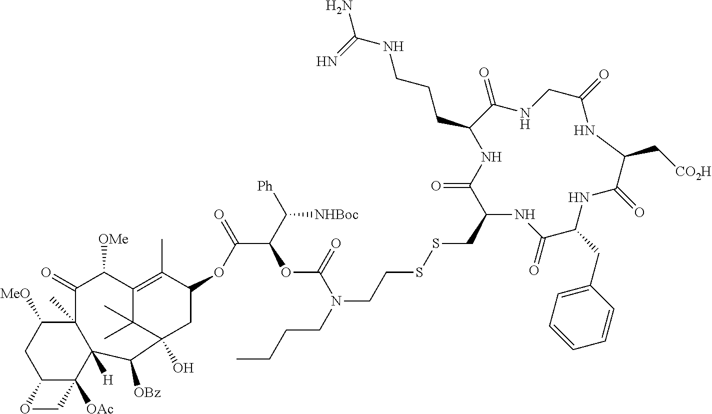

Preparation of the Conjugate

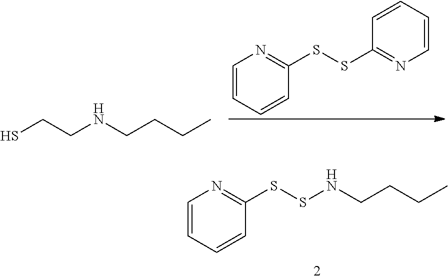

To a solution of 2,2′-dipyridyl disulfide (1.51 g, 6.85 mmol) in methanol (20 mL) was added 2-(butylamino)ethanethiol (500 μL, 3.38 mmol). The reaction was stirred at room temperature for 18 h, then the solvents removed in vacuo. The remaining material was purified by silica gel chromatography to give disulfide 2 (189 mg, 0.780 mmol, 23% yield) which was stored at −18° C. until use.

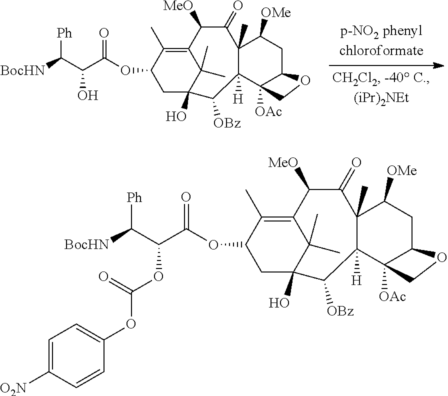

To solution of cabazitaxel (410 mg, 0.490 mmol) in dichloromethane (10 mL) and pyridine (0.50 mL), cooled to −40° C., was added a solution ofp-nitrophenyl chloroformate (600 mg, 2.98 mmol) in dichloromethane (10 mL). The reaction was stirred at −40° C. for 2 h, and the reaction warmed to room temperature and washed with 0.1N HCl (20 mL). The aqueous layer was extracted with dichloromethane (2×20 mL), and the combined organic layers dried with MgSO₄, and the solvent removed in vacuo. The remaining material was purified by silica gel chromatography to give cabazitaxel-2′-p-nitrophenylcarbonate (390 mg, 0.390 mmol, 80% yield.)

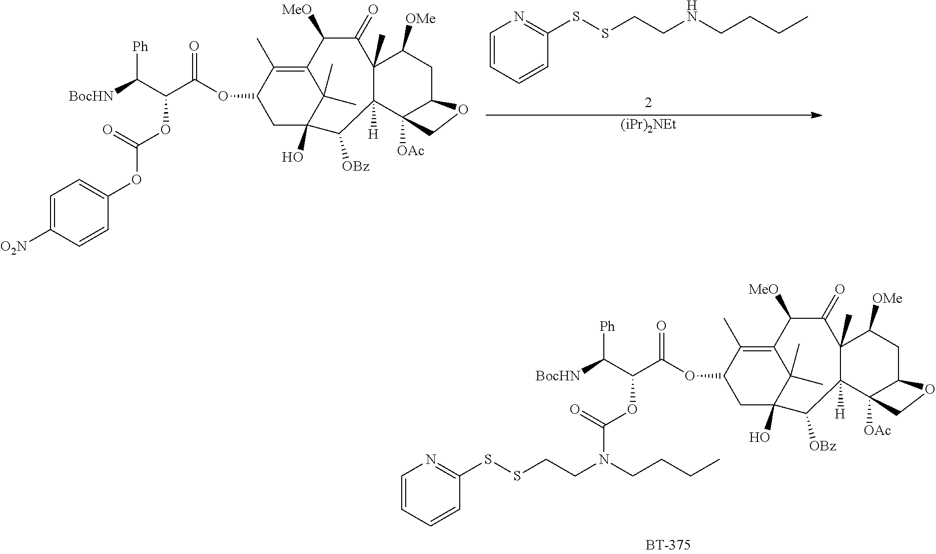

A solution of cabazitaxel-2′-p-nitrophenylcarbonate (390 mg, 0.390 mmol) in dichloromethane (15 mL) was added to 2 (190 mg, 0.784 mmol). N,N-diisopropylethylamine (1.0 mL, 5.74 mmol) was added, and the reaction stirred at 30° C. for 18 h, then the solvents removed in vacuo and the remaining material purified by silica gel chromatography to give BT-375 (326 mg, 0.295 mmol, 78% yield). ESI MS: calc'd 1103.4, found 1103.9 [M+1].

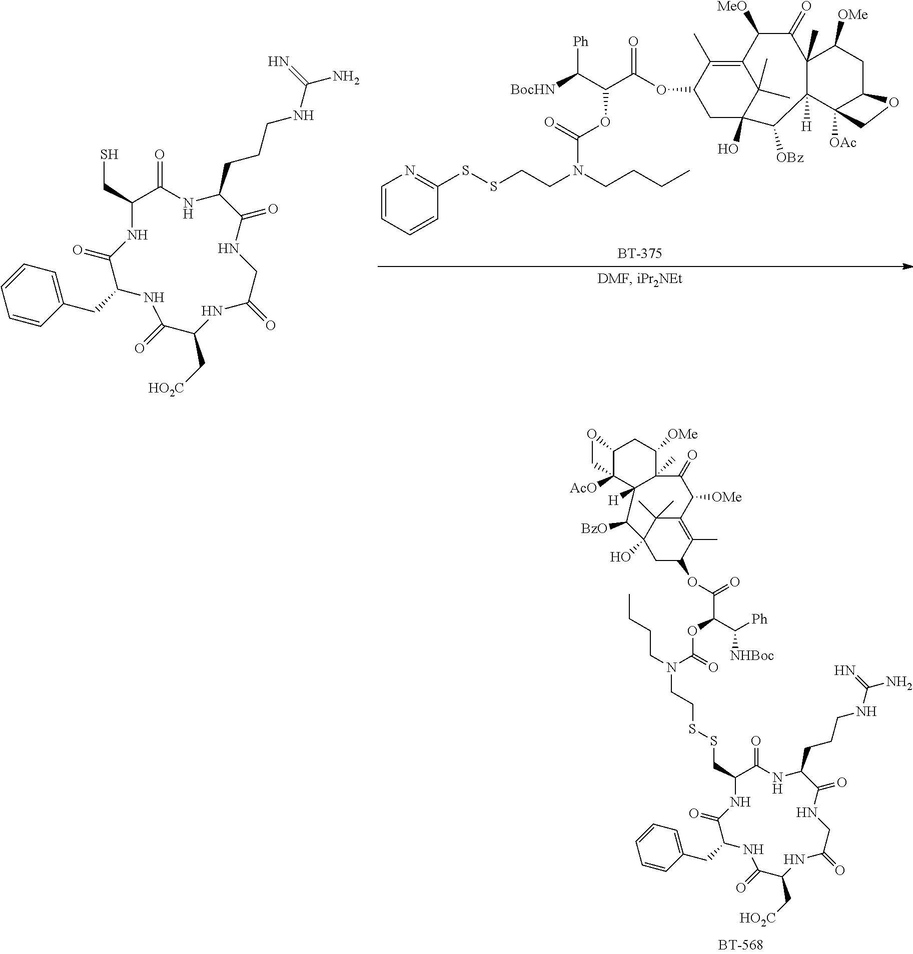

A vial was charged with cyclo(RGDfC) (66.0 mg, 0.114 mmol) and BT-375 (121 mg, 0.110 mmol). DMF (2 mL) and diisopropylethylamine (100 μL) were added, the reaction stirred at room temperature for 30 min, and the reaction loaded onto a 40 g C18 Isco column. Elution with 5% to 95% acetonitrile in water with 0.2% acetic acid provided BT-568 (71.0 mg, 0.0452 mmol, 41% yield).

### Example 3

### Preparation of Cabazitaxel-RGD Encapsulated Nanoparticles

Cabazitaxel-RGD (arginine-glycine-aspartic acid peptide) conjugate was synthesized (refer to synthesis of cabazitaxel-RGD conjugate in Example 2) and successfully encapsulated in a copolymer using a single oil in water emulsion method (refer to Table 1 below). Specifically PLA74-b-PEGS copolymer was dissolved with ethyl acetate to achieve the desired total solids concentration. The copolymer/solvent solution was added to the cabazitaxel-RGD conjugate to achieve the desired active concentration. The oil phase was then slowly added to the continuously stirred aqueous phase containing an emulsifier (such as Tween® 80) at 10/90% v/v oil/water ratio and a coarse emulsion was prepared using a rotor-stator homogenizer or an ultrasound bath. The coarse emulsion was then processed through a high-pressure homogenizer (operated at 10,000 psi) for N=2 passes to form a nanoemulsion. The nanoemulsion was then quenched by a 10-fold dilution with cold (0-5° C.) water for injection quality water to remove the major portion of the ethyl acetate solvent resulting in hardening of the emulsion droplets and formation of a nanoparticle suspension. Tangential flow filtration (500 kDa MWCO, mPES membrane) was used to concentrate and wash the nanoparticle suspension with water for injection quality water (with or without surfactants). A lyoprotectant (e.g. 10% sucrose) was added to the nanoparticle suspension and the formulation was sterile filtered through a 0.22 μm filter. The formulation was stored frozen at ≦−20° C. Particle size (Z-avg.) and the polydispersity index (PDI) of the nanoparticles were characterized by dynamic light scattering, as summarized in the table below. The actual drug load was determined using HPLC. Encapsulation efficiency was calculated as the ratio between the actual and theoretical drug load.

TABLE 1
Cabazitaxel-RDG conjugate nanoparticles in vitro and in vivo
characterization

| Formulation | NP 1 |
|---|---|
| Polymers | 100% |
| | PLA₇₄mPEG₅ |
| Polymer Conc, | 86 |
| mg/ml, | Ethyl acetate |
| Solvent | |
| Process | Emulsion |
| Emulsifier/ | 0.2% Tween |
| Stabilizer | 80 |
| Z-ave, PDI | 75, 0.09 |
| Target Drug Load | 8.5 |
| (TDL), % | |
| Actual Drug Load | 4.5 |
| (ADL), % | |
| EE % (ADL/TDL) | 53 |
| % Drug release | NA |
| at 2 h/24 h | |
| AUC_{NP}/AUCₛₒₗᵤₜᵢₒₙ | NA |

NA—not available
EE—encapsulation efficiency

### Example 4

### Pharmacokinetics of Cabazitaxel-RGD Nanoparticles

Nanoparticles are typically formulated in 10% sucrose and free drug formulations varied, but are typically dosed in 10% SOLUTOL®/10% sucrose, or physiological saline.

For PK studies, a 0.1 mg/mL solution was dosed at 10 mL/kg such that a 1 mg/kg IV bolus dose was introduced by tail vein injection into rats Following compound administration, blood was collected at 0.083 h, 0.25 h, 0.5 h, 1 h, 2 h, 4 h, 8 h, and 24 h post dose into lithium heparin coated vacuum tubes. Tubes were inverted for 5 minutes and then placed on wet ice until centrifuged for 5 minutes at 4° C. at 6000 rpm. Plasma was harvested, frozen at -80° C. and shipped to for bioanalysis on dry ice.

50 uL of rat plasma were precipitated with 300 uL of DMF and the resulting supernatant was measured for compound content by LC-MS/MS electrospray ionization in the positive mode.

This analysis indicated that the nanoparticle formulation demonstrated a significantly greater AUC of 11.6 μM*hr versus 5.3 μM*hr for the compound dosed without a nanoparticle.

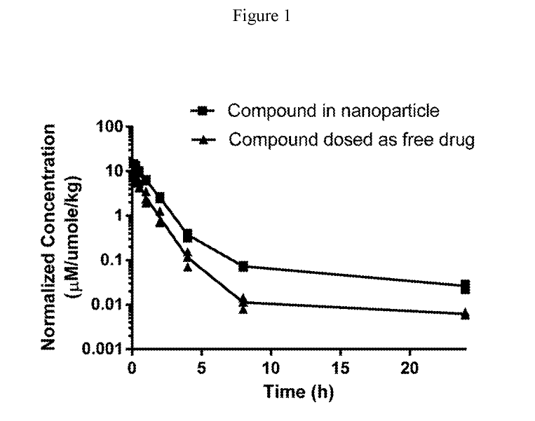

Also, this study demonstrated the better tolerability of the nanoparticle formulation. After a 1 mg/kg dose, lethargy and labored breathing were observed immediately post dose in all three rats when the free drug was administered, and one of the three animals died. For the nanoparticle formulation, no indications of toxicity were observed. See FIG. 1.

### Example 5

### Synthesis of an Octreotide-Cy5.5 Conjugate

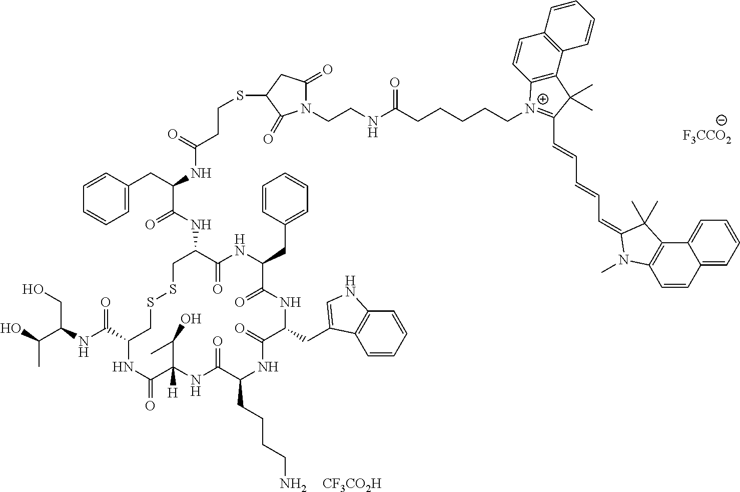 [SMILES: C[C@@H](O)[C@@H]1NC(=O)[C@H](CCCCN)NC(=O)[C@@H](Cc2c[nH]c3ccccc23)NC(=O)[C@H](Cc2ccccc2)NC(=O)[C@@H](NC(=O)[C@@H](Cc2ccccc2)NC(=O)CCSC2CC(=O)N(CCNC(=O)CCCCC[N+]3=C(/C=C/C=C/C=C4/N(C)c5ccc6ccccc6c5C4(C)C)C(C)(C)c4c3ccc3ccccc43)C2=O)CSSC[C@@H](C(=O)N[C@H](CO)[C@@H](C)O)NC1=O ; "CF3CO2H" ; "F3CCO2"]

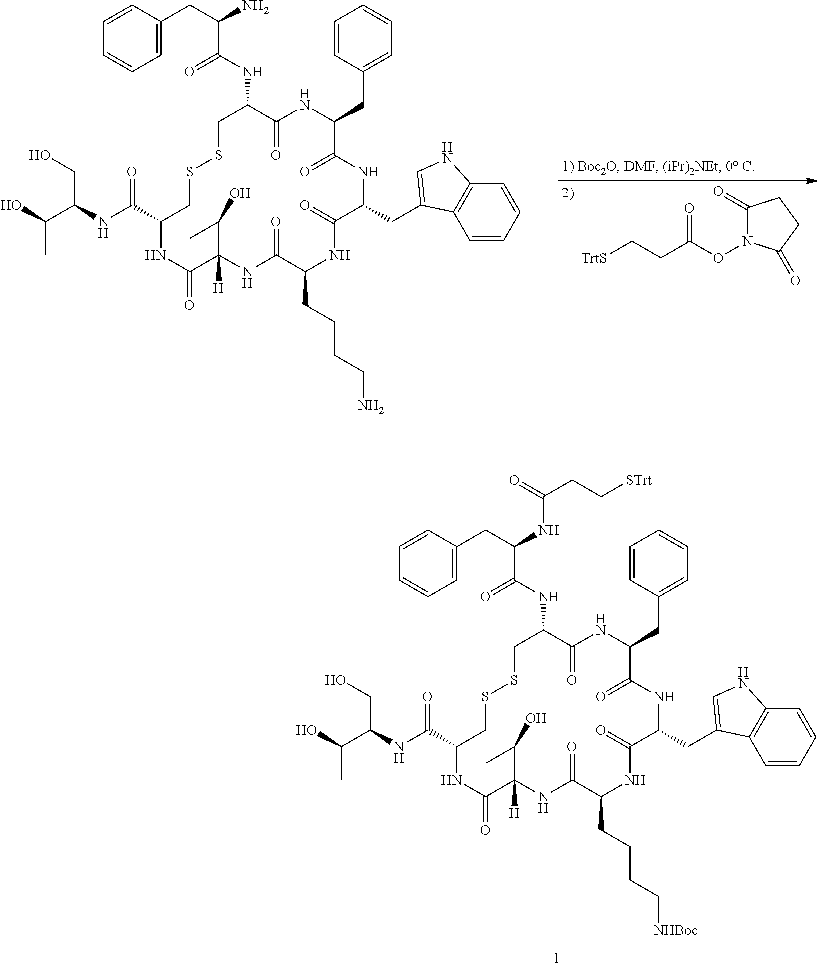

To a solution of octreotide acetate (540 mg, 0.501 mmol) in DMF (8 mL) and N,N-diisopropylethylamine (175 uL, 1.00 mmol), cooled to 0° C., was added a solution of di-tent-butyl dicarbonate (109 mg, 0.499 mmol) in DMF (7 mL). The reaction was stirred at 0° C. for 1 h, then at room temperature for 1 h. S-trityl-3-mercaptopropionic acid N-hydroxysuccinimide ester (668 mg, 1.50 mmol) was then added as a solid, and the reaction stirred at room temperature for 16 h. The solvents were removed in vacuo, and the remaining material purified by silica gel chromatography (0% to 8% methanol in dichloromethane) to give 1 (560 mg, 0.386 mmol, 77% yield).

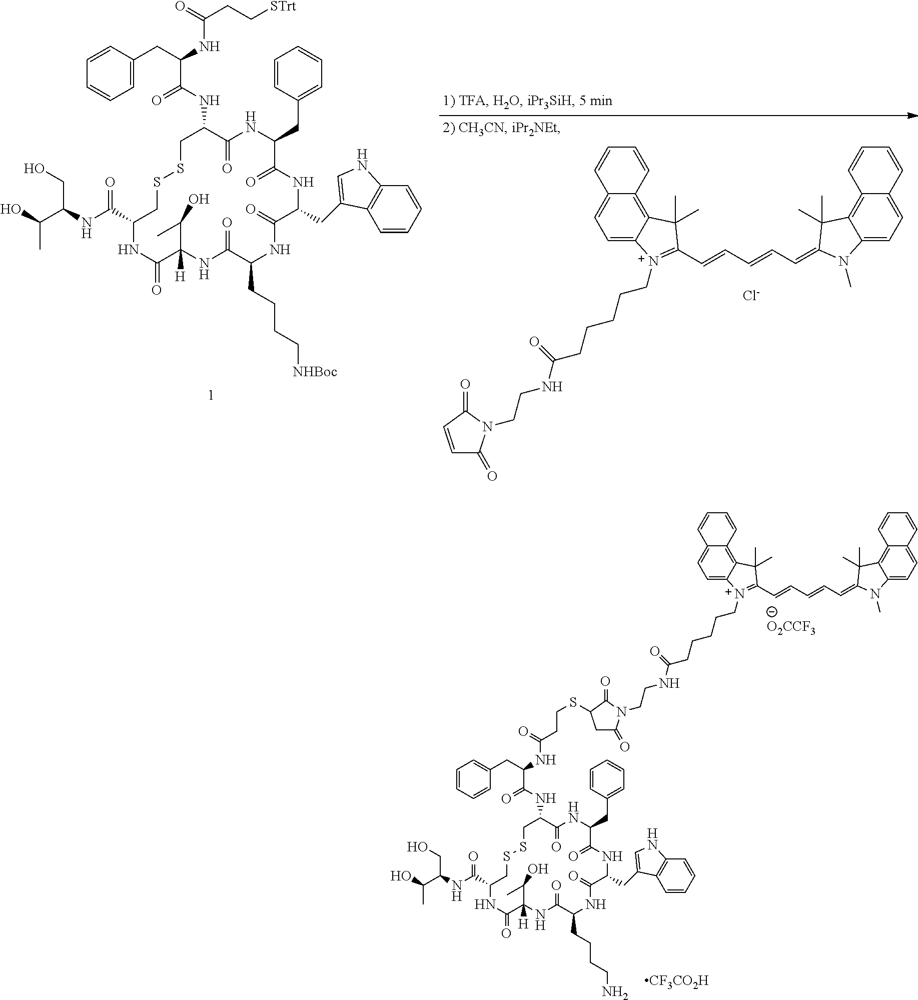

A vial was charged with 1 (58.0 mg, 0.0400 mmol), and water (60 uL) was added, followed by trifluoroacetic acid (3.0 mL). Triisopropylsilane (30 μL) was added, and the reaction stirred until the reaction turned colorless, and all solvent was removed in vacuo. The remaining residue was dissolved in acetonitrile (4.0 mL), and Cy5.5 maleimide (33.0 mg, 0.0445 mmol) was added. Diisopropylethylamine (400 μL) was added, and the reaction was stirred at room temperature for 30 min. DMF (2 mL) was added to the reaction mixture to solubilize any remaining solid material, and the reaction mixture purified by preparative HPLC (30% to 85% acetonitrile in water with 0.1% trifluoroacetic acid) to give the conjugate as a trifluoroacetate salt (24.2 mg, 0.0119 mmol, 30% yield). ESI MS: calc'd 1811.8, found 906.5 [(M+1)/2].

### Example 6

### Preparation of Octreotide-Cy5.5 Encapsulated Nanoparticles

Octreotide-Cy5.5 conjugate (Compound BT-558) was synthesized (refer to synthesis of Octreotide-Cy5.5 conjugate in Example 5) and successfully encapsulated in polymeric nanoparticles using a single oil in water emulsion method (refer to Table 2 below). Specifically, PLA74-b-PEGS, or PLA35-b-PEGS copolymers were co-dissolved with PLA57 in ethyl acetate to achieve the desired total solids concentration. The octreotide-Cy5.5 conjugate was made lipophilic by using an hydrophobic ion-pairing (HIP) technique. The conjugate has 2 positively charged moieties, one on the lysine amino acid and the other on the Cy5.5 dye. Two negatively charged dioctyl sodium sulfosuccinate (AOT) molecules were used for every 1 molecule of the conjugate to form the HIP. The conjugate and the AOT were added to a methanol, dichloromethane and water mixture and allowed to shake for 1 hour. After further addition of dichloromethane and water to this mixture, the octreotide-Cy5.5/AOT HIP was extracted from the dichloromethane phase and dried. The polymer/solvent solution was added to the octreotide-Cy5.5 conjugate to achieve the desired active concentration. The oil phase was then slowly added to the continuously stirred aqueous phase containing an emulsifier (such as Tween 80) at 10/90% v/v oil/water ratio and a coarse emulsion was prepared using a rotor-stator homogenizer or an ultrasound bath. The coarse emulsion was then processed through a high-pressure homogenizer (operated at 10,000 psi) for N=4 passes to form a nanoemulsion. The nanoemulsion was then quenched by a 10-fold dilution with cold (0-5° C.) water for injection quality water to remove the major portion of the ethyl acetate solvent resulting in hardening of the emulsion droplets and formation of a nanoparticle suspension. Tangential flow filtration (500 kDa MWCO, mPES membrane) was used to concentrate and wash the nanoparticle suspension with 0.2% Tween 80/water for injection quality water (with or without surfactants). A lyoprotectant (e.g., 10% sucrose) was added to the nanoparticle suspension and the formulation was sterile filtered through a 0.22 μm filter. The formulation was stored frozen at ≦−20° C. Particle size (Z-avg.) and the polydispersity index (PDI) of the nanoparticles were characterized by dynamic light scattering, as summarized in the table below. The actual drug load was determined using HPLC and UV-Vis absorbance. Encapsulation efficiency was calculated as the ratio between the actual and theoretical drug load.

TABLE 2
Cabazitaxel-RDG conjugate nanoparticles in vitro and
in vivo characterization

Formulation

| | NP 1 | NP 2 |
|---|---|---|
| Polymers | 50% PLA₅₇ | 50% PLA₅₇ |
| | 50% | 50% |
| | PLA₃₅mPEG₅ | PLA₇₄mPEG₅ |
| Polymer Conc, | 100 | 100 |
| mg/ml, | Ethyl acetate | Ethyl acetate |
| Solvent | | |
| Process | Emulsion | Emulsion |
| Emulsifier/ | 0.2% Tween | 0.2% Tween |
| Stabilizer | 80 | 80 |
| Z-ave, PDI | 95 (0.13) nm | 109 (0.07) nm |
| Target Drug Load | 1.12 | 1.12 |
| (TDL), % | | |
| Actual Drug Load | 0.394 | 0.21 |
| (ADL), % | | |
| EE % (ADL/TDL) | 35 | 18 |
| % Drug release | NA | NA |
| at 2 h/24 h | | |
| AUC_{NP}/AUC_{Solution} | NA | NA |

NA—not available
EE—encapsulation efficiency

### Example 7

### In vivo Characterization of Octreotide-Cy5.5 Encapsulated Nanoparticles in a Mouse Tumor Model

Imaging studies are conducted to demonstrate localization of encapsulated nanoparticles.

Six to eight week-old female NCr nude mice (Taconic, Hudson, N.Y.) mice were purchased and maintained in a pathogen-free animal facility with water and low-fluorescence mouse chow. Handling of mice and experimental procedures was in accordance with IACUC guidelines and approved veterinarian requirements for animal care and use. To induce tumor growth, mice could be implanted in the flank subcutaneous space with various human derived tumor types including SW480 (human colon adenocarcinoma cell line) and H524 (human lung cancer cell line) and tumor masses allowed to grow for 1-10 weeks. In this study, the tumor model was H69.

### In VivoFMT 4000 Tomographic Imaging and Analysis

Mice were anesthetized by isoflurane inhalation. Mice were dosed with the nanoparticle formulation of the imaging conjugate by intravenous injection.

Mice were then imaged using the FMT 4000 fluorescence tomography in vivo imaging system (PerkinElmer, Waltham, Mass.), which collected both 2D surface fluorescence reflectance images (FRI) as well as 3D fluorescence molecular tomographic (FMT) imaging datasets.

### FMT Reconstruction and Analysis

The collected fluorescence data is reconstructed by FMT 4000 system software (TrueQuant v3.0, PerkinElmer, Waltham, Mass.) for the quantification of three-dimensional fluorescence signal within the tumors and lungs. Three-dimensional regions of interest (ROI) are drawn encompassing the relevant biology.

The data demonstrate higher levels of blood and tumor fluorescence compared to normal tissue from the nanoparticle formulation containing the fluorescent targeted conjugate than the conjugate dosed without a nanoparticle formulation. There are lower levels in tissues associated with toxicity.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

We claim:

1. A polymeric controlled release nanoparticle comprising a conjugate of a chemotherapeutic agent bound via a linker to a targeting moiety,
wherein the nanoparticle is synthesized as a solid polymeric nanoparticle having a diameter of between about 10 nm to about 500 nm,
wherein no additional targeting moieties are present on the surface of the nanoparticle,
wherein, upon administration, the nanoparticle preferentially accumulates at sites of solid tumors, and
wherein the target drug load of the nanoparticle is greater than 1%.

2. The nanoparticle of claim 1, wherein the target drug load is greater than 5%.

3. The nanoparticle of claim 1, wherein the actual drug load is greater than 0.1%.

4. The nanoparticle of claim 3, wherein the actual drug load is greater than 4%.

5. The nanoparticle of claim 3, wherein the actual drug load is determined using HPLC or UV-Vis absorbance.

6. The nanoparticle of claim 1, where the encapsulation efficiency is greater than 30%.

7. The nanoparticle of claim 6, wherein the encapsulation efficiency is greater than 50%.

8. The nanoparticle of claim 6, wherein the encapsulation efficiency is calculated as the ratio between the actual and target drug load.

9. The nanoparticle of claim 1, wherein the polymer is selected from the group consisting of hydrophobic polymers, hydrophilic polymers, and copolymers thereof.

10. The nanoparticle of claim 9, wherein the hydrophobic polymers are selected from the group consisting of polyhydroxyacids, polyhydroxyalkanoates, olycaprolactones, poly(orthoesters), polyanhydrides, poly(phosphazenes), poly(lactide-co-caprolactones), polycarbonates, polyesteramides, polyesters, and copolymers thereof.

11. The nanoparticle of claim 9, wherein the hydrophilic polymers are selected from the group consisting of polyalkylene glycols, polyalkylene oxides, poly(oxyethylated polyol), poly(olefinic alcohol), polyvinylpyrrolidone), poly(hydroxyalkylmethacrylamide), poly(hydroxyalkylmethacrylate), poly(saccharides), poly(hydroxy acids), poly(vinyl alcohol), and copolymers thereof.

12. The nanoparticle of claim 9, wherein the polymer is selected from the group consisting of poly(lactic acid), poly(glycolic acid), poly(lactic-co-glycolic acid), poly(ethylene oxide), poly(ethylene glycol), poly(propylene glycol), and copolymers thereof.

13. The nanoparticle of claim 1, wherein the polymer comprises two or more different polymers.

14. The nanoparticle of claim 13, wherein the polymer comprises a poly(lactic acid) (PLA) and poly(ethylene glycol) (PEG) copolymer.

15. The nanoparticle of claim 1, wherein the particle has a diameter between 50 and 120 nm.

16. The nanoparticle of claim 1, wherein the conjugate is present in an amount between 0.1% and 10% (w/w) based upon the weight of the nanoparticle.

17. The nanoparticle of claim 1, wherein the linker is a cleavable linker.

18. The nanoparticle of claim 17, wherein the cleavable linker comprises a —S—S— group.

19. The nanoparticle of claim 17, wherein the cleavable linker is selected from the group consisting of pH-sensitive linkers, protease cleavable peptide linkers, nuclease sensitive nucleic acid linkers, lipase sensitive lipid linkers, glycosidase sensitive carbohydrate linkers, hypoxia sensitive linkers, photocleavable linkers, heat-labile linkers, enzyme cleavable linkers, ultrasound-sensitive linkers, and x-ray cleavable linkers.

20. The nanoparticle of claim 1, wherein the active agent is a small molecule, a protein, peptide, lipid, carbohydrate, sugar, nucleic acid, or combination thereof.

21. The nanoparticle of claim 1, wherein the active agent is a tyrosine kinase inhibitor or maytansine or derivative thereof.

22. The nanoparticle of claim 1, wherein the targeting moiety is selected from the group consisting of peptides and polypeptides, antibody mimetics, nucleic acids, glycoproteins, small molecules, carbohydrates, and lipids.

23. The nanoparticle of claim 1, wherein the targeting moiety targets a marker selected from the group consisting of CD19, CD70, CD56, prostate specific membrane antigen (PSMA), alpha integrin, CD22, CD138, EGFR, EphA2, AGS-5, Nectin-4, HER2, GPMNB, CD74, and Le.

24. A pharmaceutical composition comprising the nanoparticle of claim 1 and a pharmaceutically acceptable excipient.

25. A method of reducing tumor volume in a subject in need thereof comprising administering a therapeutically effective amount of the nanoparticle of claim 1.
